(19) United States
(12) Patent Application Publication
(10) Pub. No.: US 2025/0115578 A1
(43) Pub. Date: Apr. 10, 2025

(54) AROMATIC FUSED RING NAV1.8 INHIBITOR, AND USE THEREOF

(71) Applicant: CHENGDU KANGHONG PHARMACEUTICAL CO., LTD., Chengdu (CN)

(72) Inventors: Ting LIU, Chengdu (CN); Qun FANG, Chengdu (CN); Fan WU, Chengdu (CN); Zunhong KE, Chengdu (CN)

(21) Appl. No.: 18/730,175

(22) PCT Filed: Jan. 18, 2023
(86) PCT No.: PCT/CN2023/072791

§ 371 Date: Jul. 18, 2024

(30) Foreign Application Priority Data
Jan. 18, 2022 (CN) 202210055432.7
Jun. 7, 2022 (CN) 202210634203.0

(51) Int. Cl. C07D 401/14; A61K 31/55; A61P 25/04; C07D 403/04; C07D 403/14

(52) U.S. Cl. CPC C07D 401/14; A61K 31/55; A61P 25/04; C07D 403/04; C07D 403/14

(57) ABSTRACT

An aromatic fused ring compound which acts as a sodium channel blocker, and a use thereof. The aromatic fused ring compound has inhibitory activity on sodium ion channel Nav1.8, and may be used as a drug for a wide range of pain treatment.

### CROSS-REFERENCE TO RELATED APPLICATIONS

This application is the National Stage filing under 35 U.S.C. 371 of International Application No. PCT/CN2023/072791, filed on Jan. 18, 2023, which claims the benefit of CN application No. 202210055432.7, filed on Jan. 18, 2022 and CN application No. 202210634203.0, filed on Jun. 7, 2022, the contents of which are all hereby incorporated by reference herein in their entirety.

### TECHNICAL FIELD

The present invention relates to a aromatic fused ring compound with inhibitory activity to the voltage-gated sodium channel 1.8 (Nav1.8) and their use.

### BACKGROUND

Pain is a complex physiological and psychological activity and one of the most common clinical symptoms. Pain is originally a protective mechanism of the human body to alert people to potential dangers, but abnormal pain can cause physiological disorders, especially chronic pain, which seriously affects people's quality of life. According to a report of 2019 (http://news.medlive.cn/anes/info-progress/show-153086_201.html), the global prevalence of chronic pain reaches 12-30%. In the United States, the number of patients with pain has exceeded that of diabetics, cardiac patients and cancer patients, and the economic loss caused by pain is up to 560-635 billion dollars every year (https://www.physio-pedia.com/Epidemiology_of_Pain); and according to the statistics from China in 2015, the pain market also reached 20.8 billion yuan (https://paindoctor.com/resources/chronic-pain-statistics/). At present, the commonly used analgesics such as opioid receptor agonists, cyclooxygenase inhibitors, GABA receptor agonists, etc., may have some side effects such as addiction, respiratory depression, gastrointestinal reaction, or cardiovascular events, central inhibition, etc., so the clinical needs are far from being met. Thus it can be seen that the pain market has great potential. (Nora D. Volkow, A. Thomas McLellan. Opioid Abuse in Chronic Pain-Misconceptions and Mitigation Strategies. N Engl J Med, 2016, 374(13): 1253-63. Sheng H G, Shao J Y, Kir Kland S C, et al. Inhibition of human colon Cancer cell growth by selective inhibition of cyclooxygenase-2. J Chin Invest, 1997, 99: 2254. Janette Brohan, Basavana G. Goudra. The Role of GABA Receptor Agonists in Anesthesia and Sedation. CNS Drugs, 2017.)

Pain can be classified into various types based on the stimulus properties, including mechanical pain, thermal pain, and chemical pain; according to the etiology of inflammation, it can be classified into inflammatory pain and non-inflammatory pain; according to the nerve location, it can be classified into central nerve pain, peripheral nerve pain, autonomic nerve pain; and according to the course of diseases, it can be classified into acute pain and chronic pain. No matter what kind of pain, it all needs the involvement of voltage-gated sodium channels (Navs).

Human algesthesia originates from the algesiroreceptors located at peripheral nerve endings throughout the body, which can convert mechanical, thermal, cold and chemical stimuli into nerve impulses. These impulses are transmitted to the dorsal root ganglia (DRG) via the afferent nerves and then to the nerve center through the efferent nerves, thus enabling the perception of pain (Bennett D L, Clark A J, Huang J, et al. The Role of Voltage-Gated Sodium Channels in Pain Signaling. Physiol Rev, 2019, 99: 1079-1151.). Navs play the roles of triggering and transmitting signals during the transmission of nerve impulse signals, and are the main medium of the ascending branch of action potential (i.e., nerve impulse) (Mark D. Baker, John N. Wood. Involvement of Na⁺ channels in pain pathways. TRENDS in Pharmacological Sciences, 2001, 22(1): 27-31. Alan L Goldin. RESURGENCE OF SODIUM CHANNEL RESEARCH. Annu. Rev. Physiol. 2001. 63:871-894.). Therefore, inhibition of Navs helps in pain relief and treatment. However, the existing Navs inhibitors such as lidocaine, carbamazepine, lamotrigine, etc., have the defects of narrow treatment window and large side effects due to non-selectivity for Navs. Therefore, people have turned to the study of selective Nav inhibitors.

Navs are a class of transmembrane ion channel proteins composed of a subunits with molecular weight 260 kDa and β subunits with molecular weight 30˜40 kDa (Bennett D L, Clark A J, Huang J, et al. The Role of Voltage-Gated Sodium Channels in Pain Signaling. Physiol Rev, 2019, 99: 1079-1151.) According to the inhibitory activity of tetrodotoxin (TTX), the subtypes of Navs can be divided into two categories: TTX-sensitive type (TTX-S), including Nav1.1, Nav1.2, Nav1.3, Nav1.4, Nav1.6 and Nav1.7; TTX-resistant type (TTX-R), including Nav1.5, Nav1.8, Nav1.9. According to the available physiological and pharmacological studies (Bennett D L, Clark A J, Huang J, et al. The Role of Voltage-Gated Sodium Channels in Pain Signaling. Physiol Rev, 2019, 99: 1079-1151. Alan L Goldin. RESURGENCE OF SODIUM CHANNEL RESEARCH. Annu. Rev. Physiol. 2001. 63:871-94. Laura Sole, Michael M. Tamkun. Trafficking mechanisms underlying Nav channel subcellular localization in neurons. Channels, 2020, 14(1), 1-17. Manuel de Lera Ruiz, Richard L. Kraus. Voltage-Gated Sodium Channels: Structure, Function, Pharmacology and Clinical Indications. J. Med. Chem., 2015, 58(18): 7093-7118.), as shown in Table 1, Nav1.1, Nav1.2 and Nav1.3 are mainly distributed in the CNS region and are related to epilepsy, local anesthesia and other CNS diseases. Nav1.4 is mainly distributed in skeletal muscle, and its inhibitors are used as local anesthetics for myotonia. Nav1.5 is mainly distributed in cardiomyocytes, and its inhibitors are used to treat arrhythmias. Nav1.6 is involved in movement disorders; and currently, the main pain-related targets are Nav1.7, Nav1.8 and Nav1.9. Among them, the application in pain of Nav1.7 inhibitors is most widely studied, but there have been no successful clinical trials so far. There are few researches on Nav1.9, and its mechanism of action in pain is not very clear, and there are no pharmacodynamic models of pain for related inhibitors having been reported as evidences. Regarding the action mechanism of Nav1.8, Dib-Hajj et al., (Bennett D L, Clark A J, Huang J, et al. The Role of Voltage-Gated Sodium Channels in Pain Signaling. Physiol Rev, 2019, 99: 1079-1151.) summarized existing researches in their 2019 review and concluded that Nav1.8 is the main contributor to the ascending branch of action potential. Its rapid initiation supports high-frequency discharge, and it has a high activation threshold, and a slow dynamic process. Blocking Nav1.8 can block the generation of action potential and the transmission of electrical signals. Blair and Bean's research (Blair N T, Bean BP.Roles of tetrodotoxin (TTX)-sensitive Na⁺ current, TTX-resistant Na⁺ current, and Ca²⁺ current in the action potentials of nociceptive sensory neurons. J Neurosci 2002, 22: 10277-10290.) suggests that although Nav1.8 and Nav1.9 are both expressed in DRG, Nav1.8's contribution to TTX-R current is the most significant. In the acute stage of nerve injury, Nav1.8 is down-regulated in the injured neurons, but up-regulated in the adjacent undamaged neurons, thus increasing spontaneous discharge. In chronic phase, crosstalk is formed between injured neurons and undamaged neurons, resulting in the upregulation of Nav1.8 in injured neurons, thus further increasing and maintaining the idiopathic discharge. In addition to mechanistic studies, the efficacy of Nav1.8 inhibitors in animal models of pain has also been validated: For example, A-803467 developed by Abbott, had a pain relief effect exceeding 50% in carrageenan model, complete Freund's adjuvant (CFA) model, sciatic nerve chronic compression pain (CCI) model, spinal nerve ligation pain (SNL) model and acute mechanical pain model, when compared with the model group (Michael F. Jarvis, Prisca Honore, et al. A-803467, a potent and selective Nav1.8 sodium channel blocker, attenuates neuropathic and inflammatory pain in the rat. PNAS, 2007, 104(20): 8520-8525.); Moreover, A-803467 by systemic administration was superior to lidocaine in streptozotocin (STZ)-induced diabetic neuropathic heat pain model, and the two were equivalent when dosing by local injection of plantar, but the efficacy of A-803467 lasted longer (Mert, Gunes Y. Antinociceptive activities of lidocaine and the nav1.8 blocker a803467 in diabetic rats. J Am Assoc Lab Anim Sci. 2012; 51(5):579-585.); Pfizer's PF-01247324 also demonstrated significant pain relief on CFA and SNL models (Payne C E, Brown A R, Theile J W, et al. A novel selective and orally bioavailable Nav 1.8 channel blocker, PF-01247324, attenuates nociception and sensory neuron excitability. Br J Pharmacol. 2015; 172(10):2654-2670.). Most importantly, VX150, a highly selective Nav1.8 inhibitor developed by Vertex, has been clinically successful in three pain-related phase II trials. Taken together, Nav1.8 is a promising target for the treatment of pain or pain-related disorders.

TABLE 1
Overview of Navs subtypes

| | | | Major tissue | Relationship | |
|---|---|---|---|---|---|
| Targets | Genes | Type | distribution | to pain | Indications |
| Nav1.1 | SCN1A | TTX-s | CNS, PNS | / | Seizures, potential |
| | | | | | local anesthetics |
| Nav1.2 | SCN2A | TTX-s | CNS | / | Epilepsy |
| Nav1.3 | SCN3A | TTX-s | CNS(embryonic | Expressed | Epilepsy, local |
| | | | period), | rapidly when | anesthesia |
| | | | DRG(when | PNS damaged, | |
| | | | injured) | overexcitating | |
| | | | | the damaged | |
| | | | | neurons | |
| Nav1.4 | SCN4A | TTX-s | Skeletal muscle | / | Local anesthetics |
| | | | | | for myotonia |
| Nav1.5 | SCN5A | TTX-r | Cardiomyocytes | / | Arrhythmia |
| Nav1.6 | SCN8A | TTX-s | CNS, PNS | / | Dyskinesia |
| Nav1.7 | SCN9A | TTX-s | PNS | Multiple | Hereditary |
| | | | | mutants, | erythromelalgia (>20 |
| | | | | lower | mutations), |
| | | | | pain threshold | paroxysmal extreme |
| | | | | | pain disorder (>10 |
| | | | | | mutations), small fiber |
| | | | | | neuropathy, diabetic |
| | | | | | neuropathic pain |
| Nav1.9 | SCN11A | TTX-r | PNS | Lower | Inflammatory pain, |
| | | | | pain | familial sporadic pain, |
| | | | | threshold | erythromelalgia, |
| | | | | | megacolon disease |
| Nav1.8 | SCN10A | TTX-r | PNS | Lower pain | Neuropathic pain, |
| | | | | threshold and | inflammatory pain, |
| | | | | discharge | postoperative pain |
| | | | | repeatedly | (Vertex Phase II |
| | | | | | clinical) |

Currently, not many companies are reported to be developing Nav1.8 inhibitors. The main foreign companies are Abbott, Pfizer (WO2013114250A1), Gilead (AU2015224425A1), Sumitomo (WO2015008861A1), AbbVie (WO2016149169A1), Raqualia (WO2020138271A1), Merck Sharp & Dohme (WO2020092187A1), Lieber (WO2020014243A1) and Vertex (WO2019014352A1). In China, there are mainly Hengrui (WO2020151728A1) and Shanghai Jiyu Pharmaceutical (CN111808019A). Among them, the specific inhibitory activity of Nav1.8 was not disclosed in their patents, or the activity was not satisfactory. Only Pfizer's PF-04531083 and Vertex's VX-150 and VX-548 entered phase II clinical trials, and the development of PF-04531083 was terminated because it did not show better activity than placebo in postoperative toothache. VX-150 was clinically successful in three phase II clinical trials, namely inflammatory pain, postoperative acute pain and neuropathic pain, providing preliminary validation of the role of Nav1.8 in pain. However, according to Vertex's official report, the development of VX150 has been terminated due to its unsatisfactory pharmacokinetics (PK). VX-548 is currently undergoing phase III clinical trials. Therefore, the development of new Nav1.8 inhibitors remains promising and necessary.

### SUMMARY

The object of the present invention is to provide aromatic fused ring compounds with inhibitory activity to the voltage-gated sodium channels, especially Nav1.8, and their use.

To achieve the above objects, the present invention provides a compound of formula I, or a solvate, a tautomer or a pharmaceutically acceptable salt thereof:

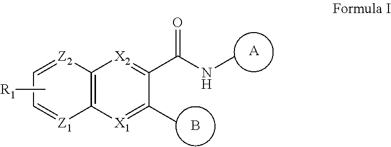

wherein X₁, X₂, Z₁, Z₂ are independently selected from substituted or unsubstituted C or N;
R₁ is independently selected from hydrogen, halogen, —NH₂, —CN, OH, —C₁-C₆ alkyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃;
wherein R₂ and R₃ are independently selected from hydrogen, —NH₂, and —C₁-C₃ alkyl;
ring A is substituted or unsubstituted benzene ring or six-membered aromatic heteroaryl;
ring B is the substituted or unsubstituted three- to ten-membered aliphatic ring or aliphatic heterocyclic ring.

In some embodiments, at least one of X₁ and X₂ is N.

In some embodiments, X₁ and X₂ are both N.

In some embodiments, X₁ is N.

In some embodiments, X₁ and X₂ are simultaneously N, and Z₁ and Z₂ are C. In some embodiments, X₁ is N, and X₂, Z₁, Z₂ are C.

In some embodiments, R₁ is selected from H, halogen. Preferably, R₁ is H.

In some embodiments, R₁ is H.

In some embodiments, ring A is selected from a six-membered aryl or heteroaryl containing 0-3 nitrogen atoms, wherein the aryl or heteroaryl is optionally substituted by hydrogen, halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃ or —POR₂R₃; where R₂ and R₃ are independently selected from hydrogen, —NH₂, —NHCH₃ and —C₁-C₃ alkyl.

In some embodiments, ring A is

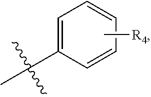

wherein R₄ is hydrogen, halogen, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃, or —POR₂R₃; R₂ and R₃ are selected independently from hydrogen, —NH₂, —NHCH₃, —C₁-C₃ alkyl, or R₂ and R₃ together with phosphorus to form a three- to eight-membered ring.

In some embodiments, ring A is

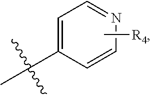

wherein R₄ is hydrogen, halogen, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃, or —POR₂R₃; R₂ and R₃ are selected independently from hydrogen, —NH₂, —NHCH₃, —C₁-C₃ alkyl, or R₂ and R₃ together with phosphorus to form a three- to eight-membered ring.

In some embodiments, ring A is

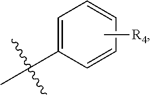

wherein R₄ is hydrogen, halogen, —SO₂R₂, —S(O)(NH)R₂, —COR₂, —CONR₂R₃; wherein R₂ and R₃ are independently selected from hydrogen, —NH₂, —NHCH₃, —C₁-C₃ alkyl.

In some embodiments, the ring A is

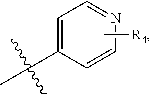

wherein R₄ is hydrogen, halogen, —SO₂R₂, —S(O)(NH)R₂, —COR₂, —CONR₂R₃; wherein R₂ and R₃ are independently selected from hydrogen, —NH₂, —NHCH₃, —C₁-C₃ alkyl.

In some embodiments, ring A is selected from:

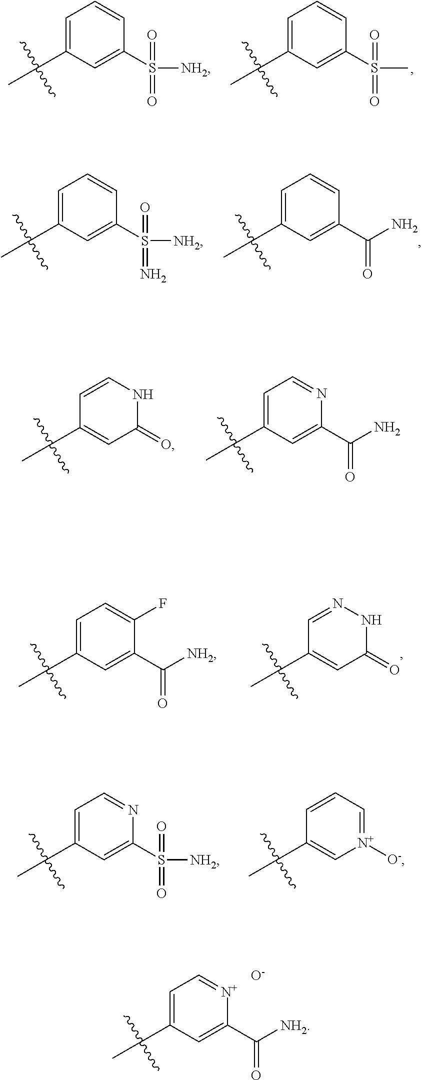

In some embodiments, ring B is selected from a three- to ten-membered aliphatic ring or aliphatic heterocyclic ring containing 0-3 heteroatoms which are selected from N, O, and S, and optionally, the aliphatic ring or aliphatic heterocyclic ring is substituted by a halogen, carbonyl group, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino, —C₃-C₆cycloalkyl and —C₃-C₆ cycloheteroalkyl.

In some embodiments, ring B is

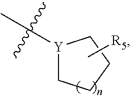

wherein Y is selected from CH or N; Ring B is substituted by m R₅, wherein R₅ is hydrogen, halogen, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino; m is an integer of 0-2 (n+3) and n is an integer of 0-6.

In some embodiments, n is an integer from 1 to 4.

In some embodiments, Y is N; n is 3; R₅ is hydrogen or halogen.

In some embodiments, ring B is:

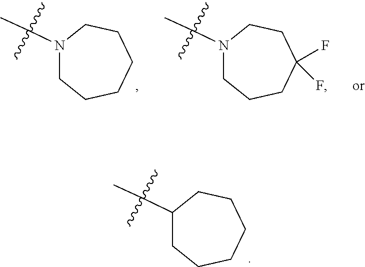 [SMILES: *N1CCCCCC1 ; *N1CCCC(F)(F)CC1 ; *C1CCCCCC1]

In some embodiments, the compounds of the present invention are described as Formula II:

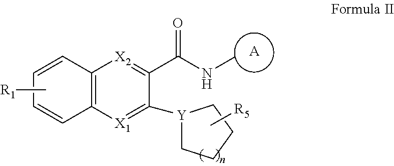

wherein X₁, X₂ are independently selected from C or N, and at least X₁ is N;
R₁ is independently selected from hydrogen, halogen, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃; wherein R₂ and R₃ are independently selected from hydrogen, —NH₂, and —C₁-C₃ alkyl;
Y is selected from CH or N;
R₅ is selected from hydrogen, halogen, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino;
n is an integer from 0 to 6;
ring A is selected from a six-membered aryl or heteroaryl containing 0-3 nitrogen atoms, wherein the aryl or heteroaryl is optionally substituted by hydrogen, halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃ or —POR₂R₃; wherein R₂ and R₃ are independently selected from hydrogen, —NH₂, —NHCH₃, and —C₁-C₃ alkyl.

In some preferred embodiments, in compound of Formula II of the present invention, ring A is

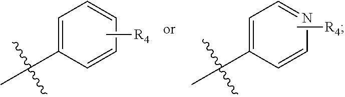

wherein R₄ is hydrogen, halogen, —NH₂, —CN, —OH, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃, or —POR₂R₃; R₂ and R₃ are selected independently from hydrogen, —NH₂, —NHCH₃, —C₁-C₃ alkyl, or R₂ and R₃ together with phosphorus to form a three- to eight-membered ring.

In some embodiments, the compounds of the present invention are shown as Formula III:

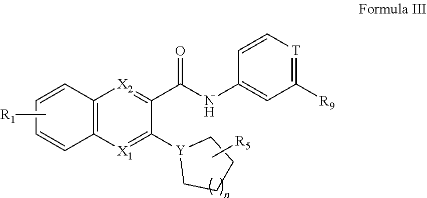

wherein X₁ and X₂ are independently selected from C or N, and at least X₁ is N;
R₁ is independently selected from hydrogen, halogen, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃; wherein R₂ and R₃ are independently selected from hydrogen, —NH₂, and —C₁-C₃ alkyl;
T is CR₆ or N;
R₆ is hydrogen, halogen or —C₁-C₆ alkyl;
R₉ is hydrogen, halogen, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃, or —POR₂R₃;
R₂ and R₃ are independently selected from hydrogen, —NH₂, —NHCH₃, —C₁-C₃ alkyl, or R₂ and R₃ together with phosphorus to form a three- to eight-membered ring;
R₅ is selected from hydrogen, halogen, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino;
n is an integer from 0 to 6.

In some preferred embodiments, in compound of Formula III of the present invention, Y is N and n is an integer of 1-4.

In some preferred embodiments, in compound of Formula III of the present invention, n is 3.

In some embodiments, the compounds of the present invention are described as formula IV:

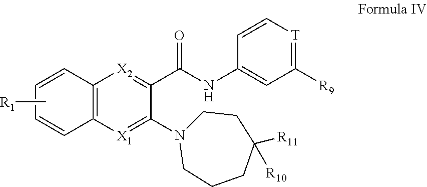

wherein R₁₀ and R₁₁ are independently selected from hydrogen or halogen;
other substituents are defined as described above.

In some preferred embodiments, in compound of Formula IV of the present invention, R₁ is independently selected from hydrogen, halogen, —C₁-C₆ alkyl, —C₁-C₆ haloalkyl, —C₁-C₆ alkoxy, —C₁-C₆ haloalkoxy.

In some preferred embodiments, in compound of Formula IV of the present invention, R₁ is independently selected from hydrogen, halogen, —CH₃ or —OCH₃.

In some preferred embodiments, in compound of Formula IV of the present invention, R₁ is selected from hydrogen or halogen.

In some preferred embodiments, in compound of Formula IV of the present invention, X₁ is N and X₂ is C.

In some preferred embodiments, in compound of Formula IV of the present invention, X₁ is N and X₂ is N.

In some preferred embodiments, in compound of Formula IV of the present invention, R₆ is hydrogen or halogen.

In some preferred embodiments, in compound of Formula IV of the invention, R₉ is —SO₂R₂, —S(O)(NH)R₂, —COR₂ or —CONR₂R₃, wherein R₂ and R₃ are independently selected from hydrogen, —NH₂, —NHCH₃, and —C₁-C₃ alkyl.

In some embodiments, the compounds of the present invention are shown as formula V:

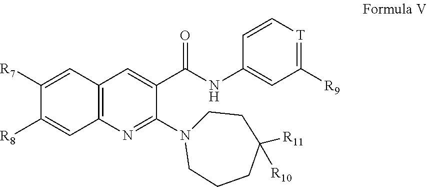

wherein R₇, R₈ are independently selected from hydrogen, halogen, —CH₃, —OCH₃;
R₉ is selected from —SO₂R₂, —S(O)(NH)R₂, —COR₂, —CONR₂R₃;
R₂ and R₃ are independently selected from hydrogen, —NH₂, —NHCH₃, —C₁-C₃ alkyl;
T is CR₆ or N;
R₁₀ and R₁₁ are independently selected from hydrogen or halogen.

In some preferred embodiments, in compound of Formula V of the present invention, R₉ is selected from —SO₂NH₂, —CONH₂.

In some preferred embodiments, in compound of Formula V of the present invention, R₉ is —SO₂NH₂.

In some preferred embodiments, in compound of Formula V of the present invention, R₇ and R₈ are independently selected from hydrogen or halogen.

In some preferred embodiments, in compound of Formula V of the present invention, R₁₀ and Ru are both halogen.

In some embodiments, the compounds of the present invention having the following general structure VI:

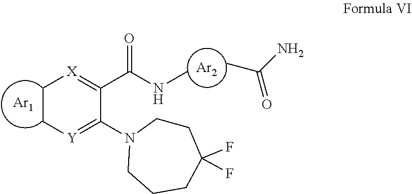

wherein Ar₁ is a substituted or unsubstituted five- or six-membered aryl or heteroaryl;
Ar₂ is a substituted or unsubstituted six-membered aryl or benzene ring;
X and Y are C or N.

In some preferred embodiments, the Ar₁ is selected from a five or six-membered aryl or heteroaryl containing 0-3 N, O, S atoms, and the aryl or heteroaryl is optionally substituted by hydrogen, halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃, or —POR₂R₃; wherein R₂ and R₃ are independently selected from hydrogen, —NH₂, —NHCH₃, and —C₁-C₃ alkyl.

In some preferred embodiments, the Ar₂ is selected from a six-membered aryl containing 0-3 N atoms or benzene ring, and the aryl or benzene ring is optionally substituted by halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino.

In some embodiments, the compounds of the present invention have the following general structure VII:

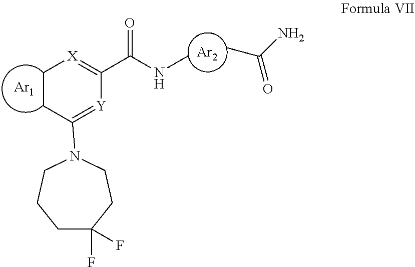

wherein Art is a substituted or unsubstituted five- or six-membered aryl or heteroaryl;
Ar₂ is a substituted or unsubstituted six-membered aryl or benzene ring;
X and Y are C or N.

In some preferred embodiments, the Ar is selected from a five or six-membered aryl or heteroaryl containing 0-3 N, O, S atoms, and the aryl or heteroaryl is optionally substituted by hydrogen, halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃, or —POR₂R₃; wherein R₂ and R₃ are independently selected from hydrogen, —NH₂, —NHCH₃, and —C alkyl₁₋₃.

In some preferred embodiments, the Ar₂ is selected from a six-membered aryl containing 0-3 N atoms or benzene ring, and the aryl or benzene ring is optionally substituted by halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino.

In some embodiments, the compounds of the present invention have the following general structure VIII:

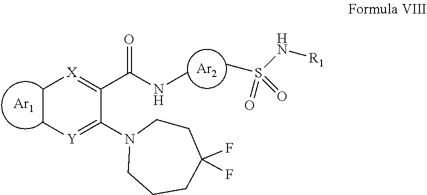

wherein Ar₁ is a substituted or unsubstituted five- or six-membered aryl or heteroaryl;
Ar₂ is a substituted or unsubstituted six-membered aryl or benzene ring;
X and Y are C or N;
R₁ is an aliphatic chain, aliphatic ring or aryl.

In some preferred embodiments, the Ar₁ is selected from a five- or six-membered aryl or heteroaryl containing 0-3 atoms of N, O, S, and the aryl or heteroaryl is optionally substituted by hydrogen, halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino.

In some preferred embodiments, the Ar₂ is selected from a six-membered aryl containing 0-3 N atoms or benzene ring, and the aryl or benzene ring is optionally substituted by halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino.

In some preferred embodiments, the R₁ is hydrogen, halogen, —NH₂, —CN, —OH, —C₁-C₆ alkyl, carbonyl, —C₁-C₆ haloalkyl, —C₁-C₆ alkoxy, —C₁-C₆ haloalkoxy or —C₁-C₆ alkylamino.

In some embodiments, the compounds of the present invention have the following general structure IX:

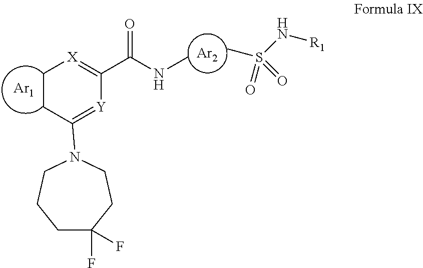

wherein Ar₁ is a substituted or unsubstituted five- or six-membered aryl or heteroaryl;
Ar₂ is a substituted or unsubstituted six-membered aryl or benzene ring;
X and Y are C or N;
R₁ is an aliphatic chain, aliphatic ring or aryl.

In some preferred embodiments, the Ar₁ is selected from a five- or six-membered aryl or heteroaryl containing 0-3 atoms of N, O, S, and the aryl or heteroaryl is optionally substituted by hydrogen, halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino.

In some preferred embodiments, the Ar₂ is selected from a six-membered aryl containing 0-3 N atoms or benzene ring, and the aryl or benzene ring is optionally substituted by halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino.

In some preferred embodiments, the R₁ is hydrogen, halogen, —NH₂, —CN, —OH, —C₁-C₆ alkyl, —C₁-C₆ haloalkyl, —C₁-C₆ alkoxy, —C₁-C₆ haloalkoxy or —C₁-C₆ alkylamino.

In some embodiments, the compounds of the present invention have the following general structure X:

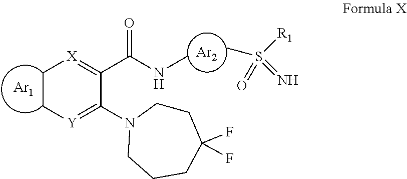

wherein Ar is a substituted or unsubstituted five- or six-membered aryl or heteroaryl;
Ar₂ is a substituted or unsubstituted six-membered aryl or benzene ring;
X and Y are C or N;
R₁ is an aliphatic chain, aliphatic ring or aryl.

In some preferred embodiments, the Ar₁ is selected from a five- or six-membered aryl or heteroaryl containing 0-3 atoms of N, O, S, and the aryl or heteroaryl is optionally substituted by hydrogen, halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino.

In some preferred embodiments, the Ar₂ is selected from a six-membered aryl containing 0-3 N atoms or benzene ring, and the aryl or benzene ring is optionally substituted by halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino.

In some preferred embodiments, the R₁ is hydrogen, —NH₂, —C₁-C₆ alkyl, —C₁-C₆ haloalkyl, —C₁-C₆ alkoxy, —C₁-C₆ haloalkoxy or —C₁-C₆ alkylamino.

In some embodiments, the compounds of the present invention have the following general structure XI:

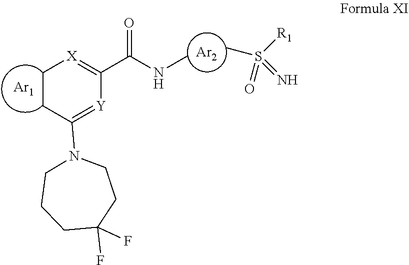

wherein Ar₁ is a substituted or unsubstituted five- or six-membered aryl or heteroaryl;
Ar₂ is a substituted or unsubstituted six-membered aryl or benzene ring;
X and Y are C or N;
R₁ is an aliphatic chain, aliphatic ring or aryl.

In some preferred embodiments, the Ar₁ is selected from a five- or six-membered aryl or heteroaryl containing 0-3 atoms of N, O, S, and the aryl or heteroaryl is optionally substituted by hydrogen, halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino.

In some preferred embodiments, the Ar₂ is selected from a six-membered aryl containing 0-3 N atoms or benzene ring, and the aryl or benzene ring is optionally substituted by halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino.

In some preferred embodiments, the R₁ is hydrogen, —NH₂, —C₁-C₆ alkyl, —C₁-C₆ haloalkyl, —C₁-C₆ alkoxy, —C₁-C₆ haloalkoxy or —C₁-C₆ alkylamino.

In some embodiments, the compounds of the present invention are selected from:

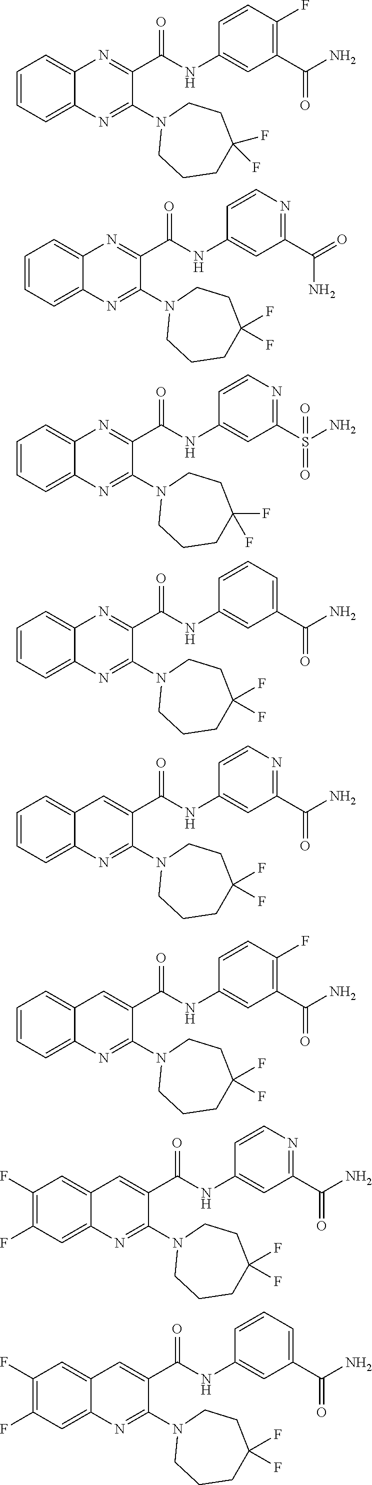

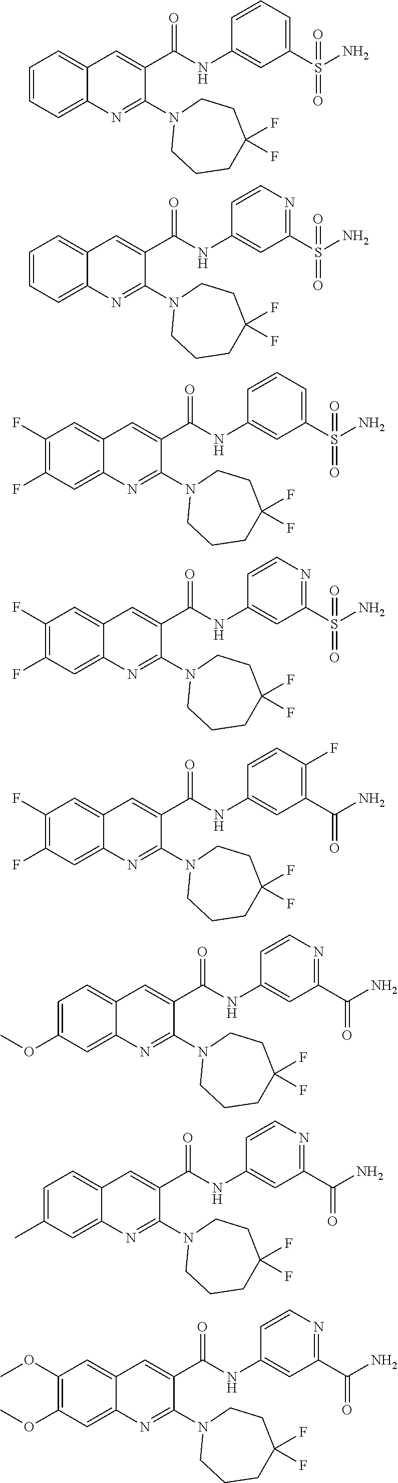

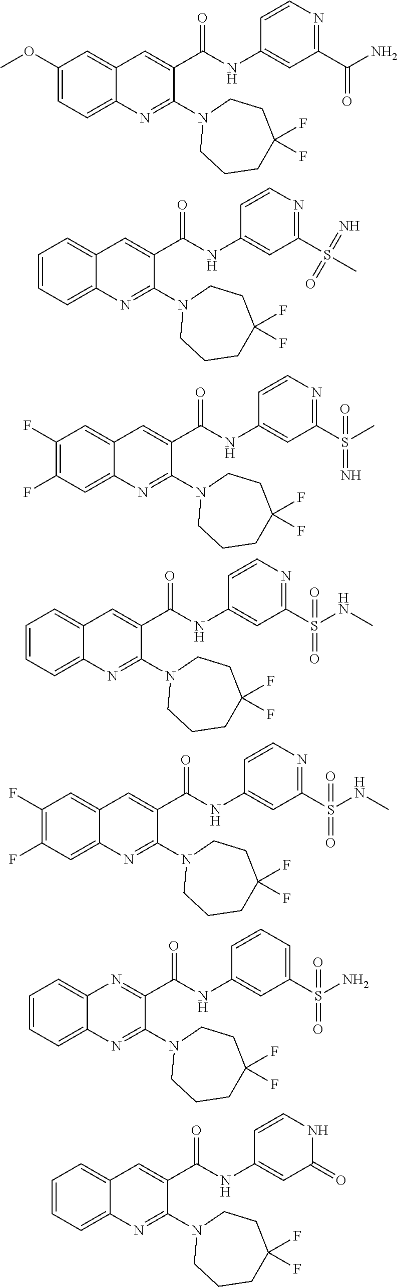

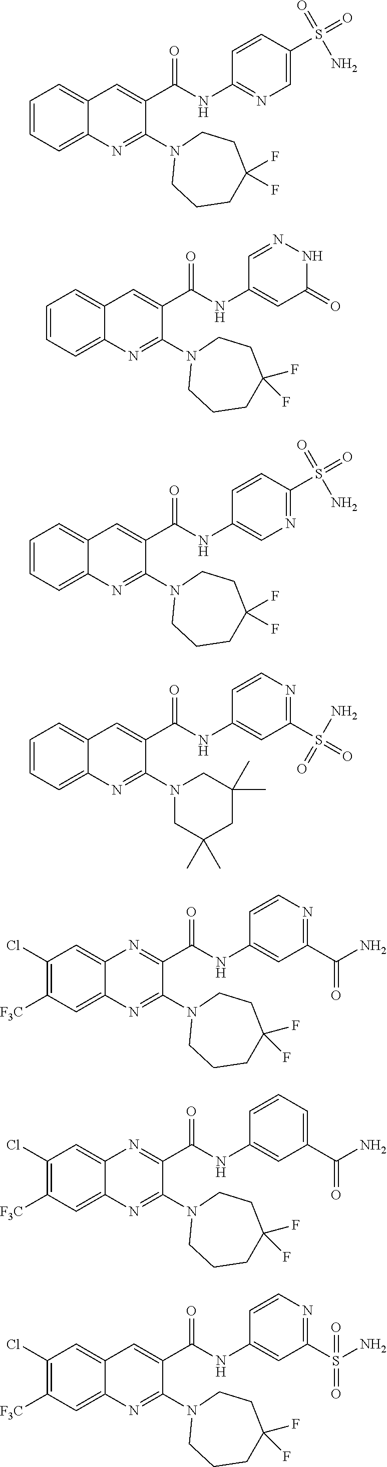

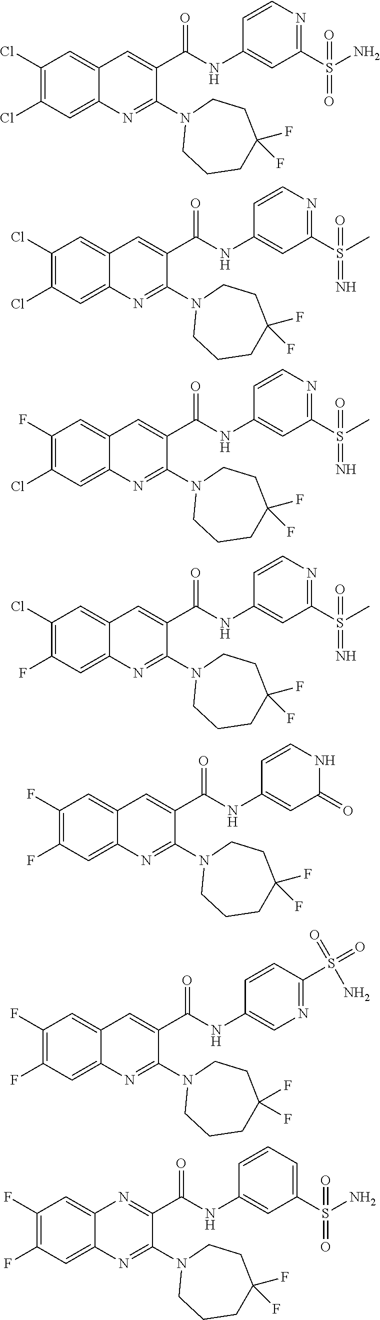

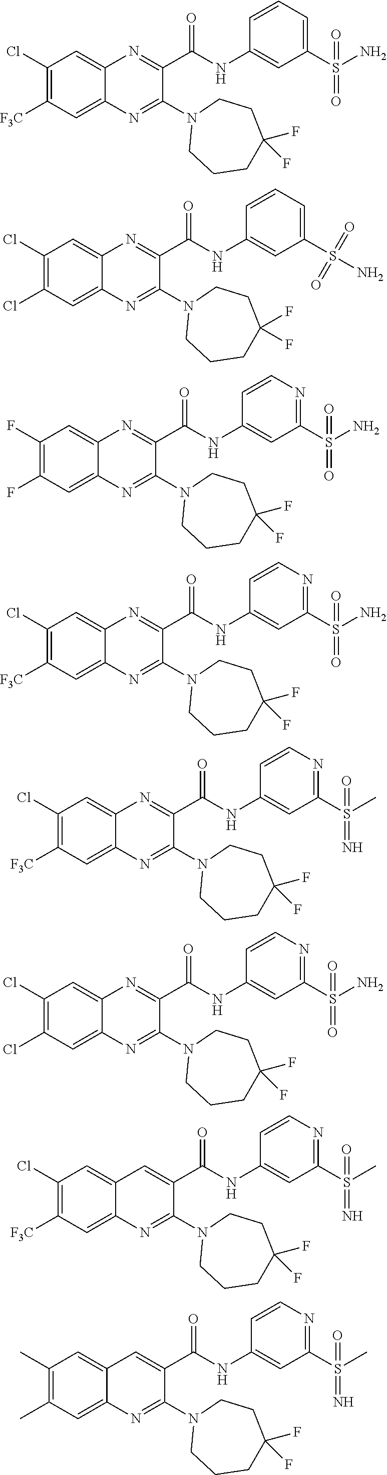

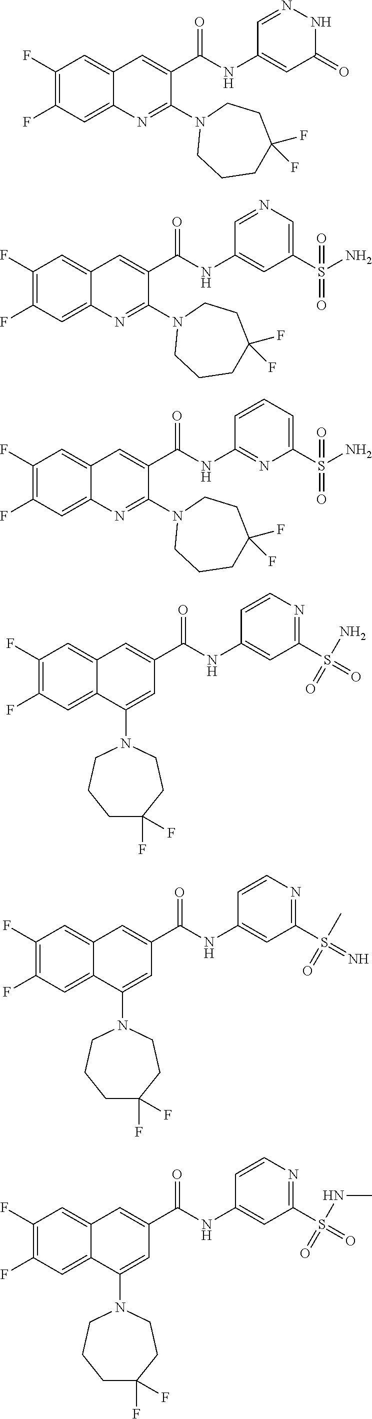

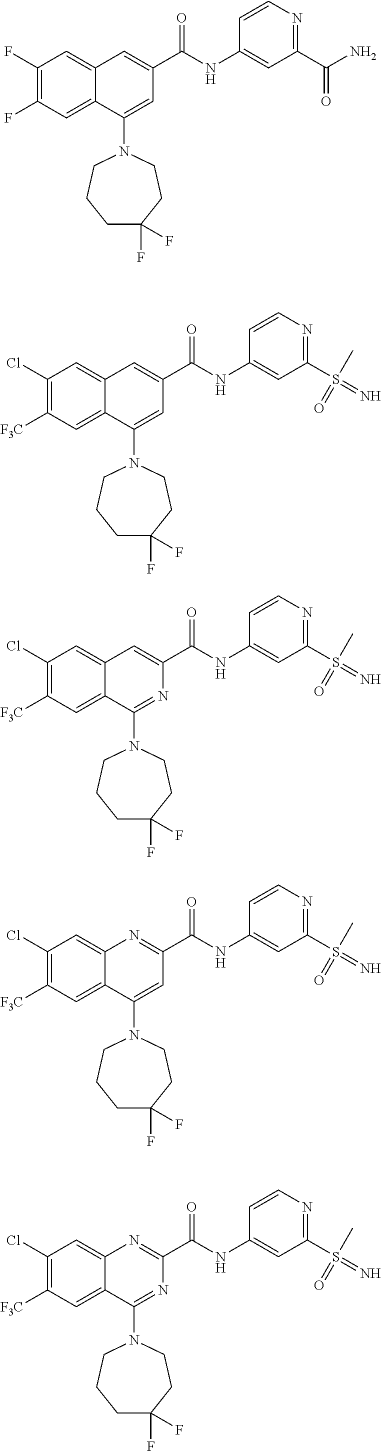 [SMILES: NC(=O)c1cc(NC(=O)c2cc(N3CCCC(F)(F)CC3)c3cc(F)c(F)cc3c2)ccn1 ; CS(=N)(=O)c1cc(NC(=O)c2cc(N3CCCC(F)(F)CC3)c3cc(C(F)(F)F)c(Cl)cc3c2)ccn1 ; CS(=N)(=O)c1cc(NC(=O)c2cc3cc(Cl)c(C(F)(F)F)cc3c(N3CCCC(F)(F)CC3)n2)ccn1 ; CS(=N)(=O)c1cc(NC(=O)c2cc(N3CCCC(F)(F)CC3)c3cc(C(F)(F)F)c(Cl)cc3n2)ccn1 ; CS(=N)(=O)c1cc(NC(=O)c2nc(N3CCCC(F)(F)CC3)c3cc(C(F)(F)F)c(Cl)cc3n2)ccn1]

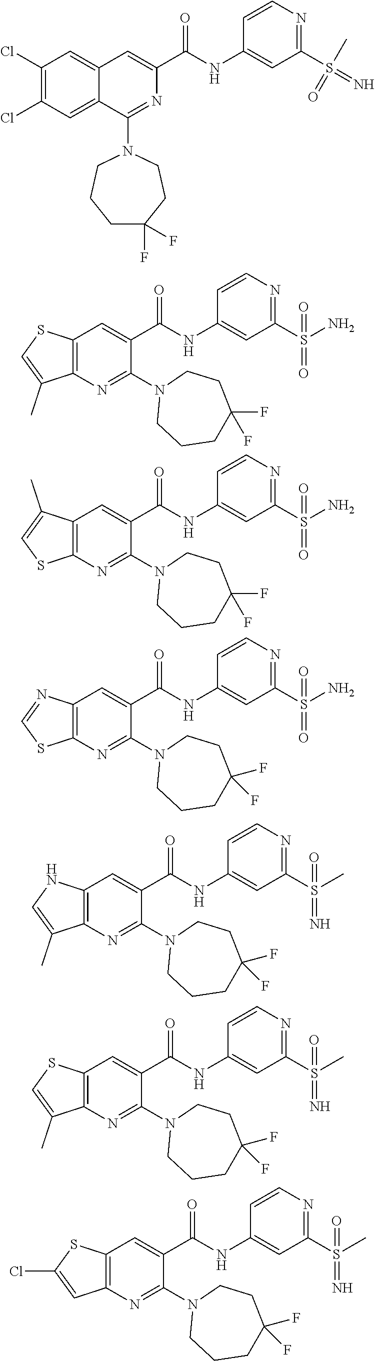

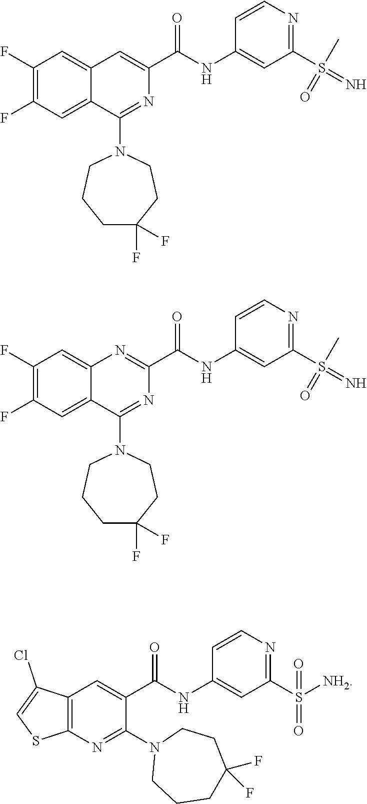 [SMILES: CS(=N)(=O)c1cc(NC(=O)c2cc3cc(F)c(F)cc3c(N3CCCC(F)(F)CC3)n2)ccn1 ; CS(=N)(=O)c1cc(NC(=O)c2nc(N3CCCC(F)(F)CC3)c3cc(F)c(F)cc3n2)ccn1 ; NS(=O)(=O)c1cc(NC(=O)c2cc3c(Cl)csc3nc2N2CCCC(F)(F)CC2)ccn1]

In some preferred embodiments, the compounds described in the present invention are selected from:

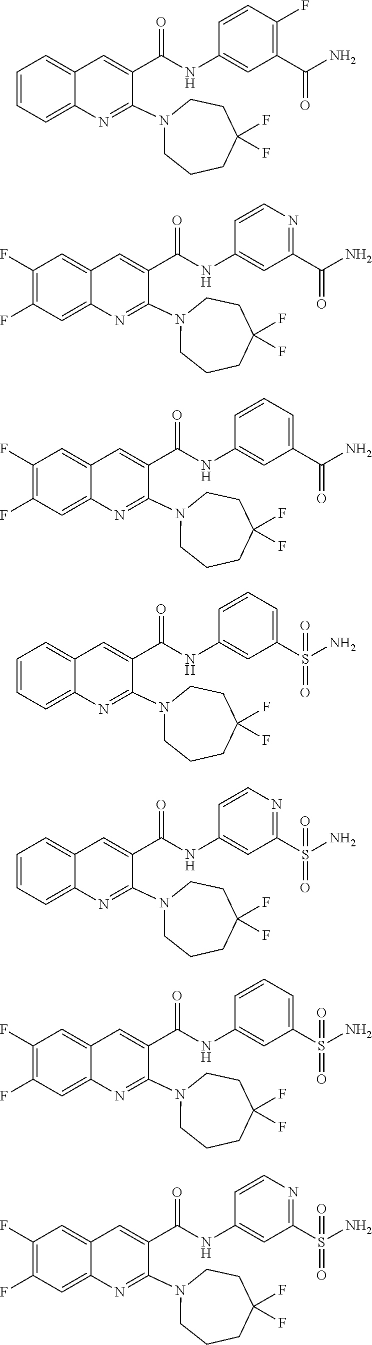

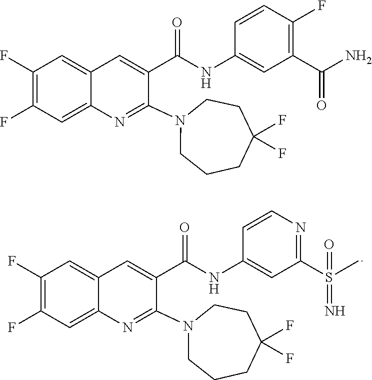 [SMILES: NC(=O)c1cc(NC(=O)c2cc3cc(F)c(F)cc3nc2N2CCCC(F)(F)CC2)ccc1F ; CS(=N)(=O)c1cc(NC(=O)c2cc3cc(F)c(F)cc3nc2N2CCCC(F)(F)CC2)ccn1]

The second object of the present invention is to provide a pharmaceutical composition comprising the compound described in the first purpose of the present invention or a pharmaceutically acceptable salt thereof, and a pharmaceutically acceptable carrier.

The third object of the invention is to provide a use of the compound or the pharmaceutically acceptable salt in the preparation of a medicament for the treatment of pain.

The fourth object of the invention is to provide a method for preventing or treating pain, comprising the administration of the compound or the pharmaceutically acceptable salt of the first object of the invention to a patient.

In some embodiments, the pain is selected from chronic pain, enteric pain, neuropathic pain, musculoskeletal pain, acute pain, inflammatory pain, cancer pain, primary pain, post-operative pain, visceral pain, multiple sclerosis, Charcot-Marie-Tooth syndrome, incontinence, or arrhythmia.

### BRIEF DESCRIPTION OF THE DRAWINGS

FIG. 1 shows the experimental voltage variation in method 2 of Example 33, wherein −80 mV is the clamping voltage, and Nav1.8 channels of the cell are in the resting state under this voltage; 10 mV is the activation voltage, and Nav1.8 channels of the cell are in the activated state under this voltage.

FIG. 2 shows mechanical allodynia test results of Example 36.

### DEFINITIONS

As used herein, the term “alkyl” by itself or as part of another substituent refers to (unless otherwise indicated) a straight or branched alkyl group having a specified number of carbon atoms (i.e., C₁₋₈ means one to eight carbons). Examples of alkyl include methyl, ethyl, n-propyl, isopropyl, n-butyl, tert-butyl, isobutyl, sec-butyl, n-amyl, n-hexyl, n-heptyl, n-octyl, etc.

As used herein, the term “heteroaryl” refers to a group having 5 to 10 heterocyclic atoms, preferably five- or six-membered monocyclic aromatic heterocycle or eight- to ten-membered bicyclic aromatic heterocycle; and a group with one to three heteroatoms in addition to carbon. A “heteroatom” is nitrogen, oxygen, or sulfur.

The term “aliphatic ring” refers to monocyclic or polycyclic alkanes with saturated or unsaturated bonds, preferably saturated monocyclic alkanes, such as cyclopropane, cyclobutane, cyclopentane, cyclohexane, cycloheptane. Polycyclic alkanes can be divided into spirals and bridged rings, depending on how they are bound.

The term “aliphatic heterocycle” refers to monocyclic or polycyclic alkanes that have saturated or unsaturated bonds of one or more heteroatoms in addition to carbon atoms. Heteroatoms such as nitrogen, oxygen, or sulfur.

The terms “substituted”, “substituted . . . by” means that any one or more hydrogen atoms bound to a particular atom are substituted by a substituent, which can include heavy hydrogen and variants of hydrogen, provided that the valence state of the particular atom is normal and the substituted compound is stable. The term “optionally substituted” means that a substituent may or may not be substituted, unless otherwise specified, the kind and number of substituents may be arbitrary on the basis of what is chemically achievable.

In addition, although the term “optionally substituted” does not appear, if any substitution occurs in a ring (such as an aliphatic ring or aryl), it should be understood that the position and number of substituents are arbitrary, as long as they are chemically achievable.

As used herein, the term “halogen” by itself or as part of another substituent means (unless otherwise stated) an atom of fluorine, chlorine, bromine or iodine. In addition, the term “haloalkyl” is intended to include both monohaloalkyl and polyhaloalkyl groups. For example, the term “C₁₋₄ haloalkyl” is intended to include trifluoromethyl, 2,2,2-trifluoroethyl, 4-chlorobutyl, 3-bromopropyl, difluoromethyl, etc.

As used herein, “alkoxy” stands for the above alkyl groups having a specific number of carbon atoms connected by an oxygen bridge, and unless otherwise specified, —C₁-C₆alkoxy groups include the alkoxy groups of C₁, C₂, C₃, C₄, C₅ and C₆. Examples of alkoxy groups include, but are not limited to: methoxy, ethoxy, n-propoxy, isopropoxy, n-butoxy, sec-butoxy, tert-butoxy, n-amoxy, and S-amoxy.

Unless otherwise specified, structures described herein are also meant to include all isomers (such as enantiomers, diastereoisomers, and geometric (or conformational) isomers) of the structure; Such as the R and S configurations of each asymmetric center, the (Z) and (E) double bond isomers, and the (Z) and (E) conformational isomers. Thus, single stereochemical isomers of these compounds and enantiomers, diastereoisomers, and geometric isomers (or conformational isomers) mixtures fall within the scope of the present invention. Unless otherwise specified, all tautomerism forms of the compounds of the invention fall within the scope of the invention. In addition, unless otherwise specified, the structures described herein are also meant to include compounds that differ only in the presence of one or more isotopically enriched atoms. For example, compounds with structures other than hydrogen being substituted by deuterium or tritium or carbon being substituted by ¹³C- or ¹⁴C-enriched carbon fall within the scope of the present invention.

Unless otherwise specified, a structure described herein is also meant to include a solvate of that structure, which refers to the physical association of the applied compound with one or more solvent molecules; The physical association involves various degrees of ionic and covalent bonding, including hydrogen bonding; In some cases, such as when one or more solvent molecules are introduced into the crystal lattice of a crystalline solid, the solvate will be able to be separated; “Solvate” covers both the solution phase and the separable solvate; Non-restrictive examples of suitable solvents include but are not limited to isopropyl alcohol, ethanol, methanol, DMSO, ethyl acetate, acetic acid and ethanolamine, etc.; “Hydrate” is a solvate in which the solvent molecule is H₂O.

As used herein, the term “composition” is intended to cover a product containing specified ingredients with specified quantity, and any product derived directly or indirectly from a combination of specified ingredients with specified quantity. The phrase “pharmaceutically acceptable” means that the carrier, diluent or excipient must be compatible with other ingredients in the formulation and not harmful to its recipients of the drug.

As used herein, the term “pharmaceutically acceptable salts” is intended to include salts that are prepared with active compounds from relatively non-toxic acids or bases. When the compounds of the present invention contain relatively acidic functional groups, alkali addition salts may be obtained by contacting the neutral form of such compounds with sufficient quantities of the desired base, either pure or in a suitable inert solvent. Examples of salts derived from pharmaceutically acceptable inorganic bases include aluminum, ammonium, calcium, copper, iron, ferrous, lithium, magnesium, manganese salts, manganous, potassium, sodium, zinc, etc. Salts derived from pharmaceutically acceptable organic bases include salts of primary, secondary, and tertiary amines (including substituted amines, cyclic amines, naturally occurring amines, etc.), Such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucosamine, histidine, isopropylamine, lysine, methylglucosamine, morpholine, piperazine, piperidine, polyamine resin, procaine, purine, theobromine, triethylamine, trimethylamine, tripropylamine, tromethamine, etc. When the compounds of the invention contain relatively basic functional groups, acid addition salts may be obtained by contacting the neutral form of such compounds with sufficient quantities of pure or desired acids in a suitable inert solvent. Pharmaceutically acceptable examples of acid addition salts include those derived from inorganic acids such as hydrochloric acid, hydrobromic acid, nitric acid, carbonic acid, monohydrocarbonic acid, phosphoric acid, monohydrophosphoric acid, dihydrophosphoric acid, sulfuric acid, monohydrosulfuric acid, hydroiodic acid or phosphite, etc., as well as salts derived from relatively non-toxic organic acids such as acetic acid, propionic acid, isobutyric acid, malonic acid, benzoic acid, succinic acid, suberic acid, fumaric acid, mandelic acid, phthalic acid, benzenesulfonic acid, p-toluenesulfonic acid, citric acid, tartaric acid, methanesulfonic acid, etc. Salts of amino acids such as arginine and organic acids such as glucuronic acid or galacturonic acid are also included.

The term “pharmaceutically acceptable carrier” means a representative carrier of any preparation or carrier medium with the capability of delivering an effective amount of the active substance of the present invention, which does not interfere with the biological activity of the active substance and has no toxic side effects to the host or patient, including water, oil, vegetable and mineral, paste base, lotion base, ointment base, etc. These substrates include suspension agents, viscosifiers, transdermal promoters, etc. Their formulations are well known to technicians in the field of cosmetics or topical drugs.

Compositions containing compounds of formula I are usually formulated according to standard pharmaceutical practice of pharmaceutical compositions. Typical preparations are prepared by mixing the compounds of the invention with diluents, carriers, or excipients. Preparations may also include buffering agents, stabilizers, surfactants, wetting agents, lubricants, emulsifiers, suspension agents, preservatives, antioxidants, opaque agents, glidants, processing aids, colorants, sweeteners, aromatics, flavorings, thinners, and one or more of the other known additives.

The compounds of the present invention may be applied in any convenient use form, such as tablets, powders, capsules, solutions, dispersions, suspensions, syrups, sprays, suppositories, gels, emulsions, patches, etc. Such compositions may comprise conventional components of pharmaceutical preparations, such as diluents, carriers, pH regulators, sweeteners, fillers, and additional active agents.

The compounds of the present invention may be administered by any suitable means, including oral, topical (including buccal and sublingual), rectum, vagina, transdermal, parenteral, subcutaneous, intraperitoneal, pulmonary, intradermal, intrathecal and epidural, and intranasal, and intralesional if required for topical treatment. Parenteral infusion includes intramuscular, intravenous, intra-arterial, intraperitoneal, intracerebral, intraocular, intrafocal, or subcutaneous administration.

“Pain” in the present invention means the pain associated with abnormalities in the voltage-gated sodium channels, in particular, the voltage-gated sodium channel is Nav1.8. Pain of the present invention includes, but is not limited to, chronic pain, intestinal pain, neuropathic pain, musculoskeletal pain, acute pain, inflammatory pain, cancer pain, primary pain, post-operative pain, visceral pain, multiple sclerosis, Charcot-Marie-Tooth syndrome, incontinence, and arrhythmia.

Among them, intestinal pain such as inflammatory bowel disease pain, Crohn's disease pain or interstitial cystitis pain. Musculoskeletal pain such as osteoarthritis pain, back pain, cold pain, burning pain or toothache. Inflammatory pain such as rheumatoid arthritis pain or vulvodynia, idiopathic pain including fibromyalgia. Neuropathic pain such as postherpetic neuralgia, diabetic neuralgia, painful HIV-associated sensory neuropathy, trigeminal neuralgia, mouth burn syndrome, amputative pain, hallucinatory pain, painful neuroma; Traumatic neuroma; Morton neuroma; Nerve entrapment injury, spinal stenosis, carpal tunnel syndrome, radiculalgia, sciatica; Nerve avulsion injury, brachial plexus avulsion injury; Complex local pain syndrome, medication-induced neuralgia, cancer chemotherapy-induced neuralgia, antiretroviral therapy-induced neuralgia; Pain after spinal cord injury, idiopathic small fiber neuropathy, idiopathic sensory neuropathy or trigeminal.

The compound of the invention is named manually or by Chemdraw software, and the commercially available compound adopts the supplier catalog name.

Compared with the prior art, the invention has the following advantages:
provides a series of structurally novel aromatic fused ring compounds that have inhibitory activity against Nav1.8 and can be used as drugs for the treatment of a wide range of pain.

### DETAIL DESCRIPTION OF THE EMBODIMENTS

The invention is further elaborated in combination with specific examples. It should be understood that these examples are used only to illustrate the invention and not to limit the scope of the invention. Experimental methods for which specific conditions are not indicated in the following examples are usually in accordance with conventional conditions or those recommended by the manufacturer. The chemical reactions described in the examples (preparation) can be readily modified to prepare many other compounds of the present invention, and alternative methods for preparing compounds of the present invention are considered to be within the scope of the present invention. Unless otherwise defined, the terms used herein have the same meaning as those familiar to those skilled in the art. In addition, any method or material similar or equivalent to the described contents may be applied in the present invention.

The structure of the compound is determined by nuclear magnetic resonance (NMR) and/or mass spectrometry (MS). The NMR shift (δ) is given in units of 10⁻⁶ (ppm). NMR was determined by a Bruker AVANCE III HD (400 MHz) or Bruker NEO (400 MHz) nuclear magnetometer with deuterated dimethyl sulphone (DMSO-d₆) or deuterated chloroform (CDCl₃) as solvent and tetramethylsilane (TMS) as internal standard. MS was determined by liquid-mass combination (LC-MS): Shimadzu's LC-20AD/LCMS-2020 or Agilent's Agilent 1260/6125. High performance liquid phase (HPLC) analysis was performed by Shimadzu's LC-20AD or LC-2030C, or Agilent's Agilent 1100/1200. Preparation and purification were performed by waters2767 or gilson GX281.

### Example 1

### Preparation of 3-(3-(4,4-difluoroazepan-1-yl)quinoxaline-2-formamido-pyridine-1-oxide (KH01)

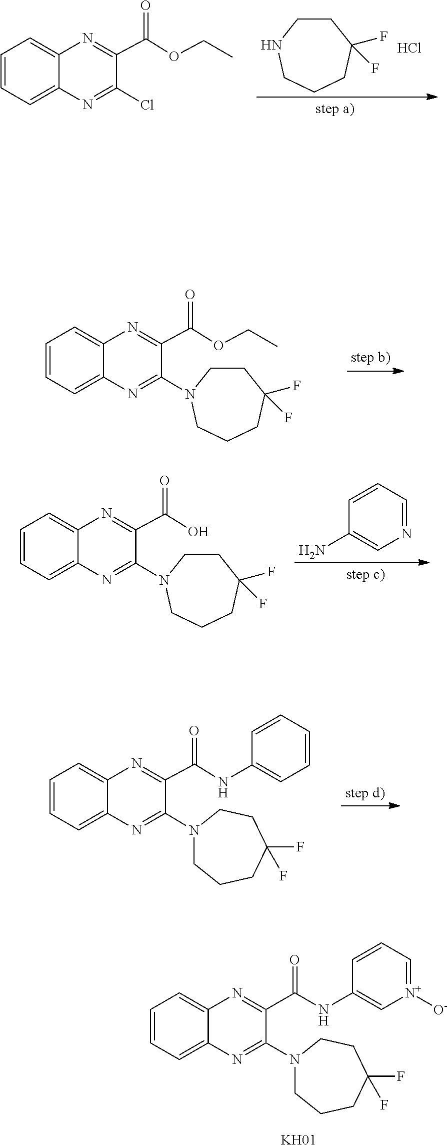

### Step a) Preparation of ethyl 3-(4,4-difluoroazepan-1-yl)-quinoxaline-2-carboxylate

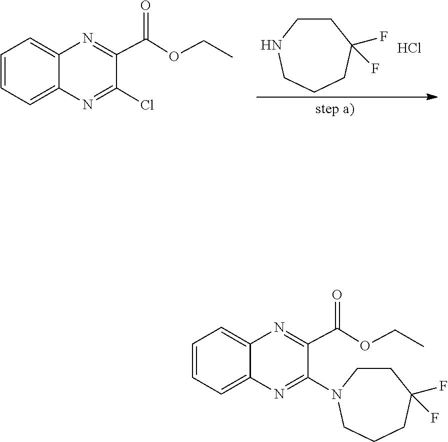

4,4-difluoroazepane hydrochloride (4.3 g, 25.4 mmol) and DIEA (8.2 g, 63.5 mmol) were added to the isopropyl alcohol (150 mL) solution of ethyl 3-quinoxaline-2-carboxylate (5 g, 21.2 mmol) and the reaction mixture was stirred at 50° C. for 16 h. After the reaction was cooled to room temperature, ethyl acetate was added to dilute it, and water was added to separate the two phases, then the organic phase was washed twice with saturated brine, dried over anhydrous sodium sulfate, filtered and concentrated under reduced pressure. The resulting crude product was purified by silica gel chromatography to obtain light yellow solid ethyl 3-(4,4-difluoroazepan-1-yl)quinoxaline-2-carboxylate (6.2 g, yield 73%). ESI-MS (m/z): 336.0 [M+H]⁺.

### Step b): Preparation of 3-(4,4-difluoroazepan-1-yl)quinoxaline-2-carboxylic acid

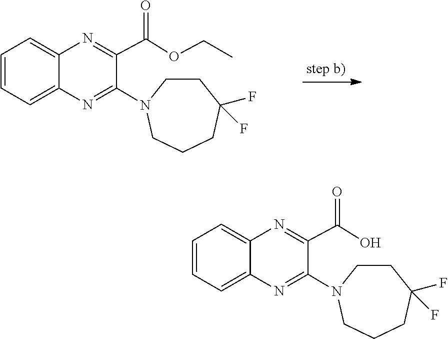 [SMILES: CCOC(=O)c1nc2ccccc2nc1N1CCCC(F)(F)CC1 ; O=C(O)c1nc2ccccc2nc1N1CCCC(F)(F)CC1]

Lithium hydroxide (3.7 g, 89.2 mmol) was slowly added to a mixture of ethyl 3-(4,4-difluoroazepan-1-yl)quinoxaline-2-carboxylate (6 g, 17.8 mmol) in water (20 mL)/methanol (60 mL)/tetrahydrofuran (5 mL), and reacted at 50° C. for 2.5 h. After the reaction was completed, it was quenched with ice water, and added 1 M of dilute hydrochloric acid to adjust the pH about 7, then organic phase was extracted with ethyl acetate, washed in saturated salt water, dried with anhydrous sodium sulfate, and filtered. After the filtrate was rotated dry, yellow solid 3-(4,4-difluoroazepan-1-yl)quinoxaline-2-carboxylic acid was obtained (4 g, yield 74%). ¹H NMR (400 MHz, DMSO-d₆) δ 7.81 (d, J=8.2 Hz, 1H), 7.67-7.55 (m, 2H), 7.47-7.34 (m, 1H), 3.78-3.72 (m, 2H), 3.64 (t, J=6.0 Hz, 2H), 2.36-2.34 (m, 2H), 2.13-1.97 (m, 2H), 1.94-1.83 (m, 2H). ESI-MS (m/z): 308.1 [M+H]⁺.

### Step c): Preparation of 3-(4,4-difluoroazepan-1-yl)-N-(pyridine-3-yl)quinoxaline-2-carboxamide

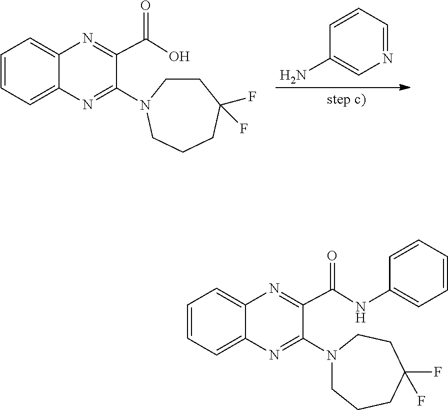 [SMILES: O=C(O)c1nc2ccccc2nc1N1CCCC(F)(F)CC1 ; Nc1cccnc1 ; O=C(Nc1ccccc1)c1nc2ccccc2nc1N1CCCC(F)(F)CC1]

3-(4,4-difluoroazepan-1-yl)quinoxaline-2-carboxylic acid (500 mg, 1.6 mmol) was dissolved in DMF (5 mL), then DIEA (420.5 mg, 3.2 mmol) and HATU (928 mg, 2.4 mmol) were added and stirred at room temperature for 1 h, then pyridine-3-amine (183.7 mg, 1.9 mmol) was added and the reaction was continued for 2 h. 10 mL saturated NH₄Cl aqueous solution was added to quench the reaction, and the resulting mixture was extracted with dichloromethane (20 mL*3), then the organic phase was separated and washed with saturated salt water, dried over anhydrous sodium sulfate, filtered, and concentrated under reduced pressure. The resulting crude product was purified by normal phase preparative chromatography to obtain yellow solid 3-(4,4-difluoroazepan-1-yl)-N-(pyridine-3-yl)quinoxaline-2-carboxamide (405 mg, yield 64.9%). ESI-MS (m/z): 382.1 [M−H]⁻.

### Step d): Preparation of 3-(3-(4,4-difluoroazepan-1-yl)) quinoxaline-2-carboxamido)pyridine-1-oxide (KH01)

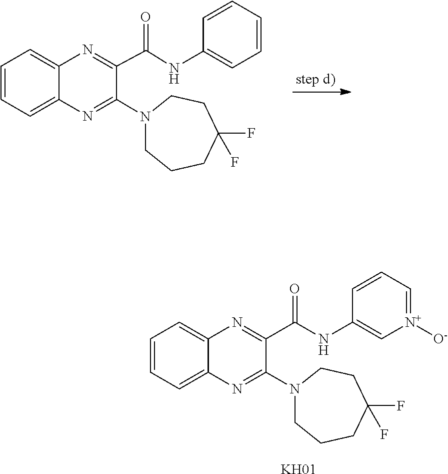

m-Chloroperbenzoic acid (180.0 mg, 1.0 mmol) was added to a solution of 3-(4,4-difluoroazepan-1-yl)-N-(pyridine-3-yl)quinoxaline-2-carboxamide (200 mg, 0.5 mmol) in dichloromethane (3 mL), and the reaction mixture was stirred at room temperature for 16 h. After the reaction was completed, 10 mL of saturated NaHCO₃ aqueous solution was added, and the resulting mixture was extracted with dichloromethane (20 mL×3), then the organic phase was separated and washed in saturated salt water, dried over anhydrous sodium sulfate, filtered, and concentrated under reduced pressure. The resulting crude product was purified by reverse preparative chromatography to obtain yellow solid 3-(3-(4,4-difluoroazepan-1-yl)quinoxaline-2-carboxamido)pyridine-1-oxide (56.42 mg, yield 27.0%). ¹H NMR (400 MHz, DMSO-d₆) δ 11.40 (s, 1H), 8.82 (t, J=1.6 Hz, 1H), 8.06 (d, J=6.4 Hz, 1H), 7.96 (d, J=8.2 Hz, 1H), 7.77-7.69 (m, 2H), 7.63 (d, J=9.4 Hz, 1H), 7.55-7.50 (m, 1H), 7.45 (dd, J=8.4, 6.4 Hz, 1H), 3.83-3.73 (m, 2H), 3.61 (t, J=5.8 Hz, 2H), 2.40 (br s, 2H), 2.03 (t, J=16.6 Hz, 2H), 1.93 (d, J=5.4 Hz, 2H). ESI-MS (m/z): 400.0 [M+H]⁺.

### Example 2

### Preparation of N-(3-carbamoyl-4-fluorophenyl)-3-(4,4-difluoroazepan-1-yl)quinoxaline-2-carboxamide (KH02)

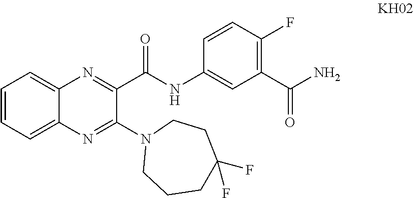 [SMILES: NC(=O)c1cc(NC(=O)c2nc3ccccc3nc2N2CCCC(F)(F)CC2)ccc1F]

N-(3-carbamoyl-4-fluorophenyl)-3-(4,4-difluoroazepan-1-yl)quinoxaline-2-carboxamide is synthesized in the same way as step a-c in Example 1, except that the pyridine-3-amine in step c was replaced with 5-amino-2-fluorobenzamide, then white solid N-(3-carbamoyl-4-fluorophenyl)-3-(4,4-difluoroazepan-1-yl)quinoxaline-2-carboxamide was obtained (67.41 mg, 35.9% yield). ¹H NMR (400 MHz, CDCl₃) δ 9.58 (s, 1H), 8.36-8.27 (m, 1H), 8.14 (dd, J=6.6, 2.9 Hz, 1H), 7.92 (dd, J=8.3, 0.8 Hz, 1H), 7.74 (d, J=7.7 Hz, 1H), 7.71-7.63 (m, 1H), 7.47-7.30 (m, 1H), 7.21 (dd, J=11.3, 9.0 Hz, 1H), 6.76 (d, J=11.3 Hz, 1H), 5.95 (s, 1H), 3.96-3.84 (m, 2H), 3.63 (d, J=5.1 Hz, 2H), 2.55-2.38 (m, 2H), 2.02 (dd, J=12.3, 8.2 Hz, 4H). ESI-MS (m/z): 444.1 [M+H]⁺.

### Example 3

### Preparation of 3-(4,4-difluoroazepan-1-yl)-N-(2-carbonyl-1,2-dihydropyridin-4-yl)quinoxaline-2-carboxamide (KH03)

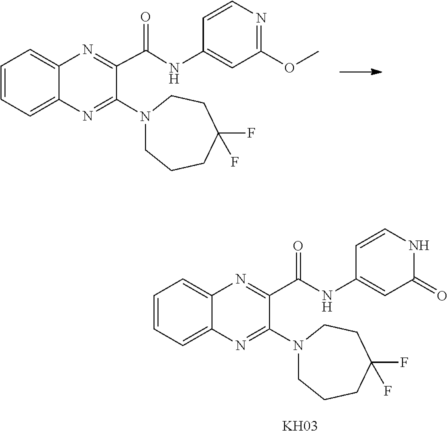

3-(4,4-difluoroazepan-1-yl)-N-(2-methoxypyridin-4-yl)quinoxaline-2-carboxamide is synthesized in the same way as step a-c in Example 1, except that the pyridine-3-amine in step c is replaced with 2-methoxypyridin-4-amine.

Sodium iodide (181.5 mg, 1.21 mmol) and trimethylchlorosilane (131 mg, 1.21 mmol) were added to a solution of 3-(4,4-difluoroazepan-1-yl)-N-(2-methoxypyridin-4-yl)quinoxaline-2-carboxamide (190 mg, 0.605 mmol) in tetrahydrofuran (4 mL), and the resulting mixture was reacted at 50° C. for 16 h. After the reaction was completed, it was quenched with water and extracted with ethyl acetate. The organic phase obtained was washed with saturated salt water, dried over anhydrous sodium sulfate, filtered, and concentrated under reduced pressure. The resulting crude product was prepared by C18 reversed columnar under the condition of formic acid to obtain light yellow solid powder 3-(4,4-difluoroazepan-1-yl)-N-(2-carbonyl-1,2-dihydropyridine-4-yl)quinoxaline-2-carboxamide (67.1 mg, yield 37%). ¹H NMR (400 MHz, DMSO-d₆) δ 11.38 (br s, 1H), 11.07 (s, 1H), 7.95 (d, J=8.2 Hz, 1H), 7.71 (d, J=3.5 Hz, 2H), 7.56-7.45 (m, 1H), 7.38 (d, J=7.1 Hz, 1H), 6.87 (s, 1H), 6.50 (d, J=7.1 Hz, 1H), 3.77 (d, J=2.5 Hz, 2H), 3.62 (br s, 2H), 2.39 (br s, 2H), 2.03 (br s, 2H), 1.93 (d, J=5.1 Hz, 2H). ESI-MS (m/z): 400.1 [M+H]⁺.

### Example 4

### Preparation of 3-(4,4-difluoroazepan-1-yl)-N-(6-carbonyl-1,6-dihydropyridazin-4-yl)quinoxaline-2-carboxamide (KH04)

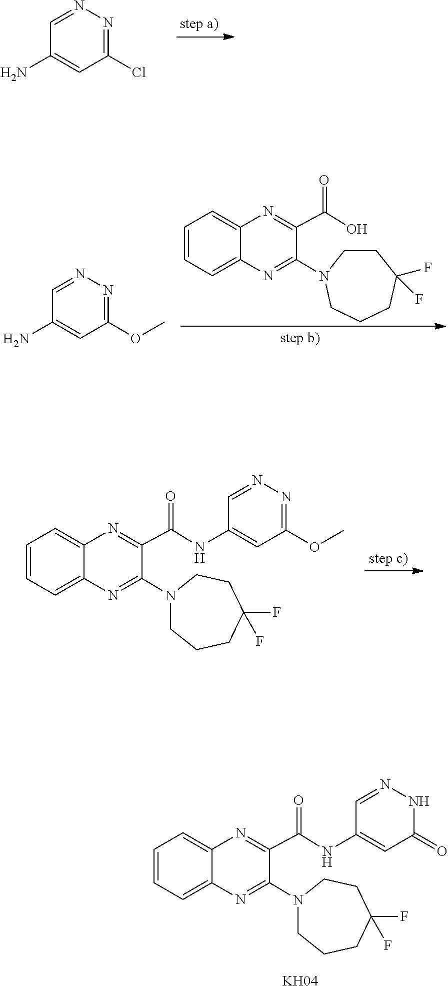

### Step a): Preparation of 6-methoxypyridazin-4-amine

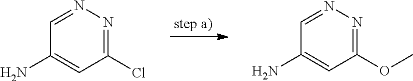

Sodium methylate (1.78 g, 32.9 mmol) was added to a solution of 6-chloro-pyridazin-4-amine (850 mg, 6.589 mmol) in methanol (20 mL) at 0° C. and stirred at 75° C. for 16 h. After the reaction mixture was cooled to room temperature, it was filtered and the filtrate was extracted and concentrated under reduced pressure. The resulting crude product was purified by silica gel column chromatography to obtain white solid 6-methoxypyridazin-4-amine (780 mg, yield 94%). ¹H NMR (400 MHz, DMSO-d₆) δ 8.26 (d, J=1.5 Hz, 1H), 6.33 (s, 2H), 5.97 (d, J=1.4 Hz, 1H), 3.87 (s, 3H). ESI-MS (m/z): 126.1 [M+H]⁺.

### Step b): Preparation of 3-(4,4-difluoroazepan-1-yl)-N-(6-methoxydrazin-4-yl)quinoxaline-2-carboxamide

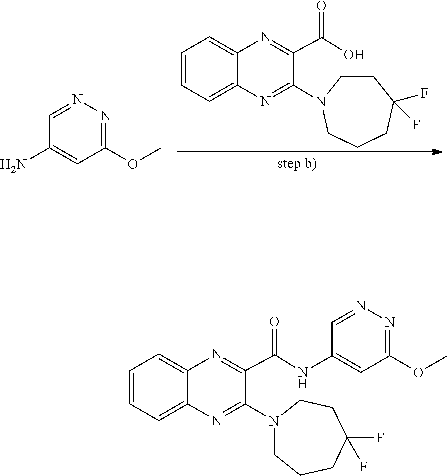

For the synthesis of 3-(4,4-difluoroazepan-1-yl)quinoxaline-2-carboxylic acid, see step a-b of Example 1.

HATU (297 mg, 0.781 mmol) and DIEA (210 mg, 1.628 mmol) were added to a solution of 3-(4,4-difluoroazepan-1-yl)quinoxaline-2-carboxylic acid (200 mg, 0.651 mmol) in DMF (5 mL). After it was reacted at room temperature for 0.5 h, 6-methoxy-pyridazin-4-amine (163 mg, 1.302 mmol) was added and reacted at room temperature for 16 h. After the reaction was completed, water was added to quench the reaction, and the organic phase (20 mL) was extracted with ethyl acetate, washed with saturated salt water, dried over anhydrous sodium sulfate, and filtered. The filtrate was concentrated under reduced pressure, and the resulting crude product was purified by silica gel plate (ethyl acetate/petroleum ether (v/v)=1/1) to obtain a light yellow oil 3-(4,4-difluoroazepan-1-yl)-N-(6-methoxy-pyridazin-4-yl)quinoxaline-2-carboxamide (55 mg, 20% yield). ¹H NMR (400 MHz, DMSO-d6) δ 11.65 (s, 1H), 9.13 (s, 1H), 7.95 (s, 1H), 7.74 (d, J=13.5 Hz, 2H), 7.63 (s, 1H), 7.52 (s, 1H), 4.05 (s, 3H), 3.77 (br s, 2H), 3.58 (t, J=5.6 Hz, 2H), 2.39 (br s, 2H), 2.00 (d, J=12.4 Hz, 2H), 1.92 (d, J=4.7 Hz, 2H). ESI-MS (m/z): 415.1 [M+H]⁺.

### Step c): Preparation of 3-(4,4-difluoroazepan-1-yl)-N-(6-carbonyl-1,6-dihydropyridazin-4-yl)quinoxaline-2-carboxamide (KH04)

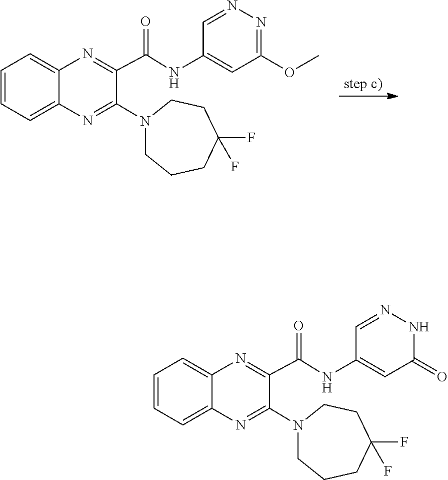 [SMILES: COc1cc(NC(=O)c2nc3ccccc3nc2N2CCCC(F)(F)CC2)cnn1 ; O=C(Nc1cn[nH]c(=O)c1)c1nc2ccccc2nc1N1CCCC(F)(F)CC1]

Sodium iodide (50 mg, 0.332 mmol) and trimethylchlorosilane (36 mg, 0.332 mmol) were added to a solution of 3-(4,4-difluoroazepan-1-yl)-N-(6-methoxydiazin-4-yl)quinoxaline-2-carboxamide (55 mg, 0.133 mmol) in tetrahydrofuran (4 mL), and that was reacted at 50° C. for 16 hours. After the reaction was completed, water was added to quench the reaction. The organic phase was extracted with ethyl acetate (20 mL), washed with saturated salt water, dried with anhydrous sodium sulfate, and filtered. The filtrate was concentrated under reduced pressure, and the resulting crude product was prepared by C18 reverse column under the condition of formic acid to obtain the light yellow solid powder 3-(4,4-difluoroazepan-1-yl)-N-(6-carbonyl-1,6-dihydropyridazin-4-yl)quinoxaline-2-carboxamide (13.2 mg, yield 25%). ¹H NMR (400 MHz, DMSO-d₆) δ 12.89 (s, 1H), 11.45 (s, 1H), 8.09 (d, J=2.3 Hz, 1H), 7.96 (d, J=8.1 Hz, 1H), 7.73-7.72 (m, 2H), 7.52 (ddd, J=8.3, 5.6, 2.8 Hz, 1H), 7.33 (d, J=2.0 Hz, 1H), 3.79-3.74 (m, 2H), 3.57 (br s, 2H), 2.40 (br s, 2H), 2.05-2.00 (m, 2H), 1.93 (br s, 2H). ESI-MS (m/z): 401.1 [M+H]⁺.

### Example 5

### Preparation of N-(2-carbamoylpyridin-4-yl)-3-(4,4-difluoroazepan-1-yl)quinoxaline-2-carboxamide (KH05)

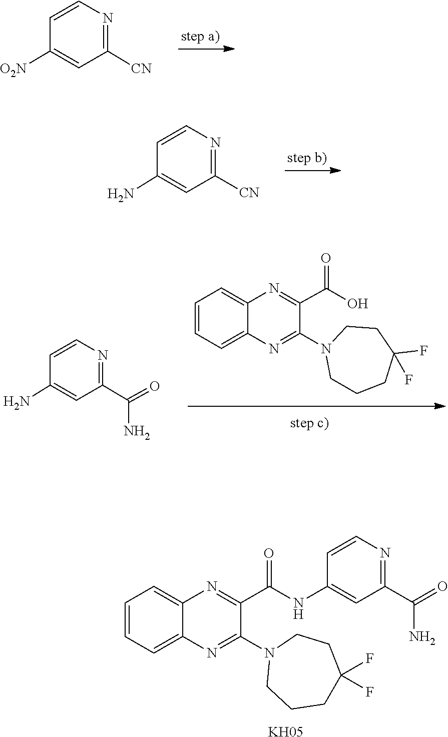

### Step a): Preparation of 4-aminopyridine-2-carbonitrile

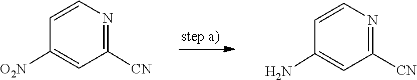 [SMILES: N#Cc1cc([N+](=O)[O-])ccn1 ; N#Cc1cc(N)ccn1]

Iron powder (1.88 g, 33.5 mmol) and ammonium chloride (1.78 g, 33.5 mmol) were added to a mixture of 2-cyano-4-nitropyridine (1 g, 6.7 mmol) in ethanol (100 mL)/water (10 mL) and stirred at 60° C. for 2 h. After the reaction mixture was cooled to room temperature, it was filtered, and the filtrate was quenched with water, then the organic phase was extracted with ethyl acetate, washed with saturated brine, dried with anhydrous sodium sulfate, filtered, and concentrated under reduced pressure. The resulting crude product was purified by silica gel column chromatography (methanol/dichloromethane (v/v)=1/40) to obtain white solid 4-aminopyridine-2-carbonitrile (400 mg, yield 51%). ¹H NMR (400 MHz, DMSO-d₆) δ 8.07 (d, J=5.7 Hz, 1H), 6.95 (d, J=2.3 Hz, 1H), 6.69 (dd, J=5.7, 2.3 Hz, 1H), 6.60 (s, 2H). ESI-MS (m/z): 120.2 [M+H]⁺.

### Step b): Preparation of 4-aminopyridine-2-carboxamide

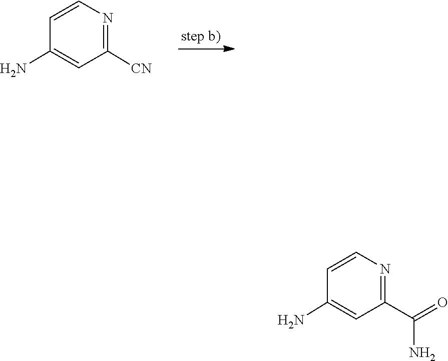 [SMILES: N#Cc1cc(N)ccn1 ; NC(=O)c1cc(N)ccn1]

Cesium carbonate (1.65 g, 10.08 mmol) was added to a solution of 4-aminopyridine-2-carbonitrile (400 mg, 3.36 mmol) in dimethyl sulfoxide (10 mL), and 30% hydrogen peroxide (0.5 mL) was slowly added at 0° C. The reaction was carried out at room temperature for 4 h. After the reaction was completed, it was quenched with ice water, and the organic phase was extracted with ethyl acetate, washed with saturated salt water, dried with anhydrous sodium sulfate, and filtered. The filtrate was concentrated under reduced pressure, and the resulting crude product was purified by reverse phase preparative chromatography to obtain 4-aminopyridine-2-carboxamide (300 mg, yield 65%). ¹H NMR (400 MHz, DMSO-d₆) δ 8.01 (d, J=5.5 Hz, 1H), 7.89 (s, 1H), 7.39 (s, 1H), 7.21 (d, J=2.3 Hz, 1H), 6.58 (dd, J=5.5, 2.4 Hz, 1H), 6.28 (s, 2H). ESI-MS (m/z): 308.1 [M+H]⁺.

### Step c): Preparation of N-(2-carbamoylpyridin-4-yl)-3-(4,4-difluoroazepan-1-yl)quinoxaline-2-carboxamide (KH05)

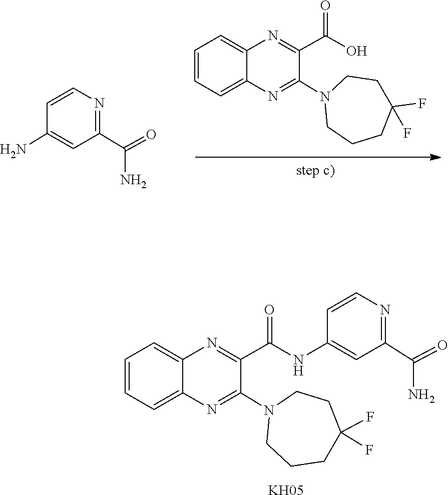

For the synthesis of 3-(4,4-difluoroazepan-1-yl)quinoxaline-2-carboxylic acid see step a-b of Example 1.

2-(7-Azobenzotriazole-1-yl)-N,N,N′,N′-tetramethyluronium hexafluorophosphate (HATU) (333 mg, 0.8795 mmol), N,N-diisopropylethylamine (188 mg, 1.459 mmol) were added to a solution of 3-(4,4-difluoroazepan-1-yl)quinoxaline-2-carboxylic acid (224 mg, 0.730 mmol) in N,N-dimethylformamide (5 mL), and the reaction mixture was reacted at room temperature for 0.5 h, and then 4-aminopyridine-2-carboxamide (110 mg, 0.803 mmol) was added, and the resulting mixture was reacted at room temperature for 16 h. After the reaction was completed, it was quenched with water, and the organic phase was extracted with ethyl acetate, washed with saturated salt water, dried with anhydrous sodium sulfate, filtered, and concentrated under reduced pressure. The resulting crude product was purified by preparative chromatography to a light yellow solid powder N-(2-carbamoylpyridin-4-yl)-3-(4,4-difluoroazepan-1-yl)quinoxaline-2-carboxamide (25.6 mg, yield 8.3%). ¹H NMR (400 MHz, DMSO-d₆) δ 11.55 (s, 1H), 8.58 (d, J=5.4 Hz, 1H), 8.45 (s, 1H), 8.13 (s, 1H), 8.03-7.91 (m, 2H), 7.77-7.71 (m, 2H), 7.68 (s, 1H), 7.55-7.48 (m, 1H), 3.88-3.76 (m, 2H), 3.63 (t, J=5.7 Hz, 2H), 2.40 (br s, 2H), 2.10-2.01 (m, 2H), 1.93 (d, J=5.4 Hz, 2H). ESI-MS (m/z): 427.1 [M+H]⁺.

### Example 6

### Preparation of 3-(4,4-difluoroazepan-1-yl)-N-(2-sulfamoylpyridin-4-yl)quinoxaline-2-carboxamide (KH06)

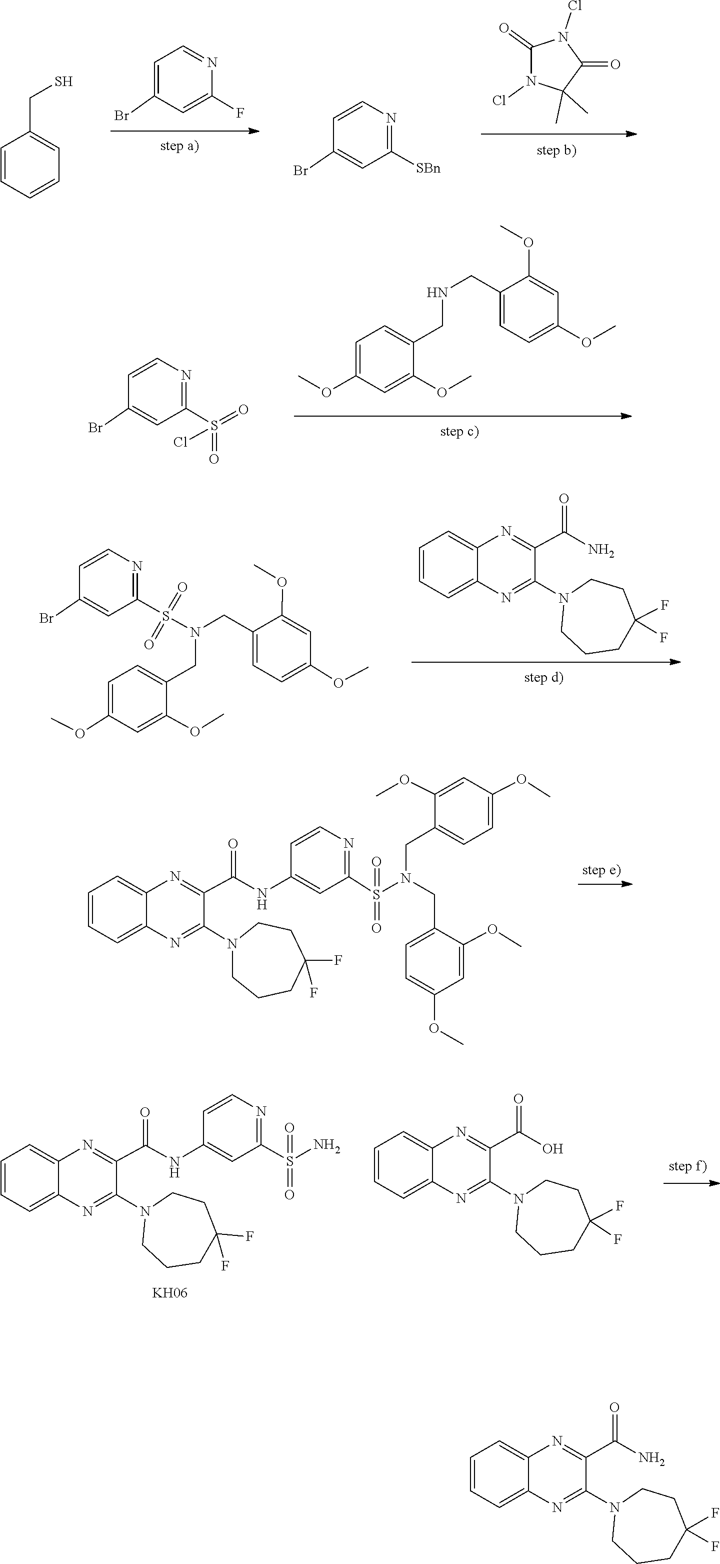

### Step a): Preparation of 2-benzylthio-4-bromopyridine

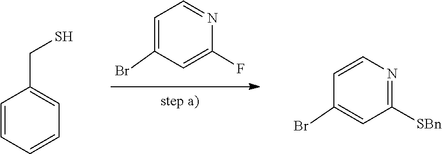

The solution of benzyl thiol (3 g, 24.1 mmol) in tetrahydrofuran (50 mL) was placed into an ice water bath, and NaH (1.45 g, 60.4 mmol, 60%) was added in batches, then the reaction system was returned to room temperature and stirred for 1 h. Then 4-bromo-2-fluoropyridine (4.5 g, 25.5 mmoL) was added and it was continued to react for 2 h. After the reaction was completed, the reaction mixture was concentrated under reduced pressure, and 20 mL saturated NH₄Cl aqueous solution was added under the condition of ice water bath, organics were extracted with dichloromethane (20 mL×3), and the organic phase was separated and washed with saturated salt water, dried with anhydrous sodium sulfate, and filtered. The filtrate was concentrated under reduced pressure, and the resulting crude product was purified by normal phase chromatography (petroleum ether/ethyl acetate (v/v)=4/1) to obtain colorless oil 2-benzylthioether-4-bromopyridine (5.8 g, yield 85.6%). ESI-MS (m/z): 281.9 [M+2]⁺.

### Step b): Preparation of 4-bromopyridine-2-sulfonyl chloride

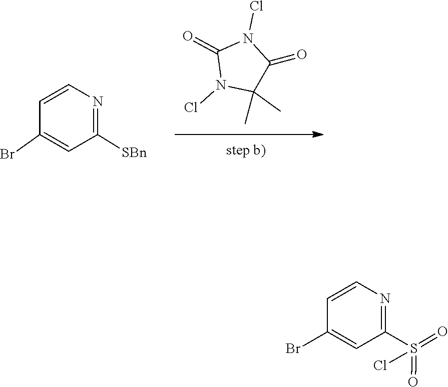

Water (8 mL) and acetic acid (4 mL) were added to a solution of 2-benzylthio-4-bromopyridine (2 g, 7.1 mmol) in dichloromethane (28 mL), and the reaction system was cooled to 0° C. 1,3-Dichloro-5,5-dimethylimidazolidine-2,4-dione (4.22 g, 21.4 mmol) was added in batches, and then the reaction system was returned to room temperature and stirred for 16 h. After the reaction was completed, the reaction system was placed into an ice water bath, and 20 mL saturated NaHCO₃ aqueous solution was added to quench the reaction. The organics was extracted with dichloromethane (20 mL×3), and the organic phase was separated and washed with saturated salt water, dried with anhydrous magnesium sulfate, and filtered. The cream-colored semi-oily 4-bromopyridine-2-sulfonyl chloride (4.78 g, crude) was obtained after concentration under reduced pressure. ESI-MS (m/z): 253.9 [M-Cl+MeO⁻+2]⁺.

### Step c): Preparation of 4-bromo-N,N-bis(2,4-dimethoxybenzyl)pyridine sulfonamide

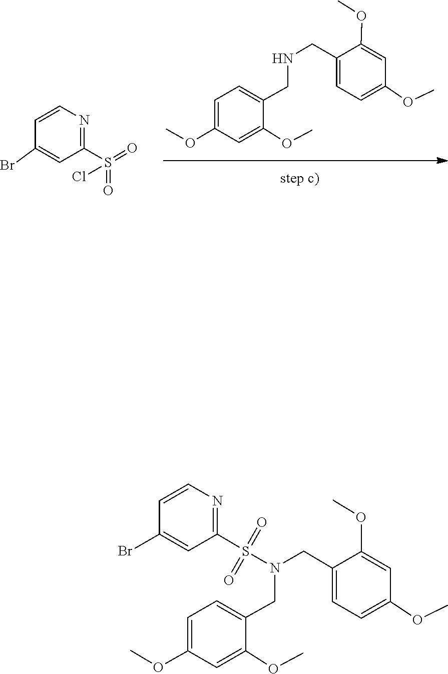

4-Bromopyridine-2-sulfonyl chloride (2 g, 7.8 mmol) was dissolved in dichloromethane (20 mL), and DIEA (5.04 g, 38.9 mmol) was added under an ice water bath, and then bis(2,4-dimethoxybenzyl) amine (3.71 g, 11.7 mmol) was added in batches. After that, the reaction system was returned to room temperature and stirred for 4 h. After the reaction was completed, 20 mL saturated NH₄Cl aqueous solution was added to quench the reaction system, and the organic phase was extracted with dichloromethane (25 mL×3), separated and washed with saturated salt water, dried with anhydrous sodium sulfate, and filtered. The filtrate was concentrated under reduced pressure. The resulting crude product was purified by normal phase chromatography (petroleum ether/ethyl acetate (v/v)=4/1), and the light brown sugar 4-bromo-N,N-bis(2,4-dimethoxybenzyl)pyridine sulfonamide was prepared (860 mg, yield 20.5%). LCMS (ESI, m/z): 560.9 [M+Na+2]⁺. ¹H NMR (400 MHz, DMSO-d₆) δ 8.51 (d, J=5.2 Hz, 1H), 7.86-7.82 (m, 1H), 7.60 (d, J=1.8 Hz, 1H), 7.08 (d, J=8.4 Hz, 2H), 6.45 (dd, J=8.4, 2.3 Hz, 2H), 6.35 (d, J=2.3 Hz, 2H), 4.43 (s, 4H), 3.73 (s, 6H), 3.61 (s, 6H). ESI-MS (m/z): 560.9 [M+Na⁺+2]⁺.

### Step d): Preparation of N-(2-(N,N-bis(2,4-dimethoxybenzyl)sulfamoyl)pyridin-4-yl)-3-(4,4-difluoroazepan-1-yl)quinoxaline-2-carboxamide

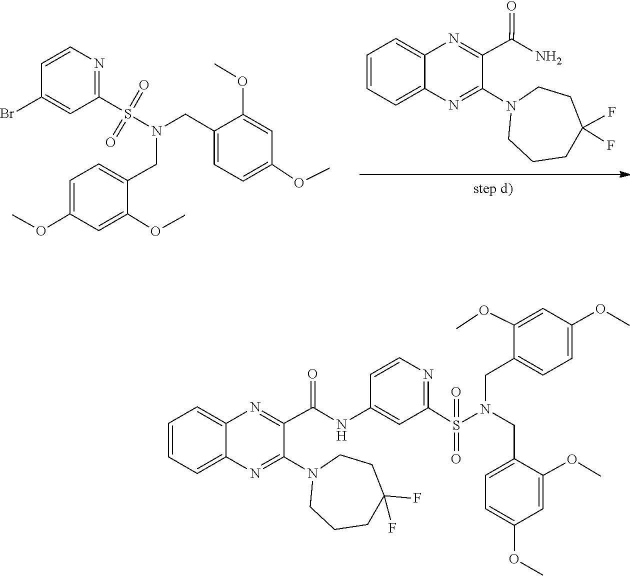

4-Bromo-N,N-bis(2,4-dimethoxybenzyl)pyridine sulfonamide (140 mg, 0.26 mmol) was dissolved in toluene (2 mL), then 3-(4,4-difluoroazepan-1-yl)quinoxaline-2-carboxamide (87.7 mg, 0.28 mmol) and Cs₂CO₃ (254.6 mg, 0.78 mmol) were added, followed by Ruphos Pd G₂ (20.2 mg, 0.026 mmol). After the reaction system was replaced with nitrogen, the reaction was carried out at 110° C. for 16 h. After the reaction was completed, the reaction system was cooled to room temperature, and then concentrated under reduced pressure. The resulting residue was added with 10 mL saturated NH₄Cl aqueous solution, and extracted with dichloromethane (25 mL×3). The organic phase was separated and washed with saturated salt water, dried with anhydrous sodium sulfate, and filtered. The filtrate was concentrated under reduced pressure. The resulting crude products was purified by the positive phase chromatography (petroleum ether/ethyl acetate (v/v)=3/1) to obtain yellow solid N-(2-(N,N-bis(2,4-dimethoxybenzyl)sulfamoy)pyridin-4-yl)-3-(4,4-difluoroazepan-1-yl)quinoxaline-2-carboxamide (116.8 mg, Yield 58.7%). ESI-MS (⁺m/z): 763.1 [M+H].

### Step e): Preparation of 3-(4,4-difluoroazepan-1-yl)-N-(2-sulfamoylpyridin-4-yl)quinoxaline-2-carboxamide (KH06)

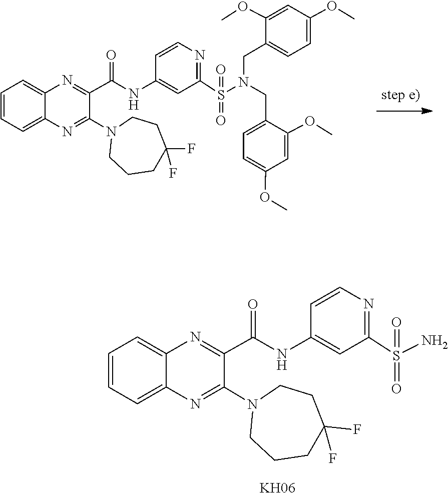

N-(2-(N,N-bis(2,4-dimethoxybenzyl)sulfamoyl)pyridin-4-yl)-3-(4,4-difluoroazepan-1-yl) quinoxaline-2-carboxamide (110 mg, 0.14 mmol) was dissolved in dichloromethane (2 mL), then trifluoroacetic acid (1 mL) was added and the reaction system was stirred at room temperature for 2 h after addition. After the reaction was completed, 10 mL saturated NaHCO₃ aqueous solution was added, and the reaction mixture was extracted with dichloromethane (25 mL×3). The organic phase was separated and washed with saturated salt water, dried with anhydrous sodium sulfate, and filtered, and the filtrate was concentrated under reduced pressure. The resulting crude product was purified by reverse phase chromatography to obtain yellow solid N-(2-aminosulfonylpyridine-4-yl)-3-(4,4-difluoroazepan-1-yl)quinoline-2-carboxamide (31.17 mg, yield 46.7%). ¹H NMR (400 MHz, DMSO-d₆) δ 11.71 (s, 1H), 8.66 (d, J=5.4 Hz, 1H), 8.40 (d, J=1.7 Hz, 1H), 7.96 (d, J=8.1 Hz, 1H), 7.93 (dd, J=5.5, 2.0 Hz, 1H), 7.77-7.71 (m, 2H), 7.55-7.51 (m, 1H), 7.50 (d, J=4.2 Hz, 2H), 3.86-3.74 (m, 2H), 3.61 (t, J=5.7 Hz, 2H), 2.40 (brs, 2H), 2.05 (d, J=19.4 Hz, 2H), 1.93 (d, J=5.4 Hz, 2H). ESI-MS (m/z): 463.0 [M+H]⁺.

### Step f): Preparation of 3-(4,4-difluoroazepan-1-yl)quinoxaline-2-carboxamide

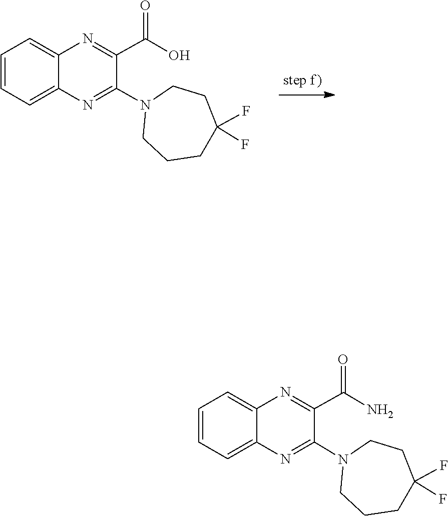 [SMILES: O=C(O)c1nc2ccccc2nc1N1CCCC(F)(F)CC1 ; NC(=O)c1nc2ccccc2nc1N1CCCC(F)(F)CC1]

For the synthesis of 3-(4,4-difluoroazepan-1-yl)quinoxaline-2-carboxylic acid, see step a-b of Example 1.

HATU (371.2 mg, 0.9 mmol) and DIEA (420.5 mg, 3.2 mmol) were added to a solution of 3-(4,4-difluoroazepan-1-yl)quinoxaline-2-carboxylic acid (200 mg, 0.6 mmol) in DMF (5 mL). After half an hour, NH₄Cl (139.2 mg, 2.6 mmol) was added and stirred at room temperature for 2 h. Water (15 mL) was added to quench the reaction, then dichloromethane (20 mL×3) was used to extract the reaction system, and the organic phase was separated and washed with saturated salt water, dried with anhydrous sodium sulfate, and filtered, and the filtrate was concentrated under reduced pressure. The resulting crude product was purified by normal phase chromatography (dichloromethane/methanol (v/v)=20/1) to obtain yellow solid 3-(4,4-difluoroazepan-1-yl)quinoxaline-2-carboxamide (160 mg, yield 80.2%). ¹H NMR (400 MHz, DMSO-d₆) δ 8.25 (brs, 1H), 7.85 (d, J=7.2 Hz, 2H), 7.65 (d, J=3.9 Hz, 2H), 7.45-7.40 (m, 1H), 3.84-3.73 (m, 2H), 3.68 (2H, t, J=5.8 Hz), 2.39 (d, J=10.3 Hz, 2H), 2.11-1.88 (m, 4H). ESI-MS (m/z): 306.9 [M+H]⁺.

### Example 7

### Preparation of N-(3-carbamoylphenyl)-3-(4,4-difluoroazepan-1-yl)quinoxaline-2-carboxamide (KH07)

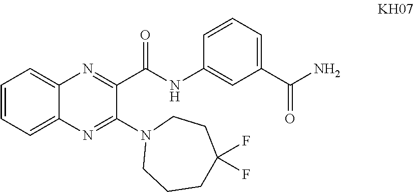 [SMILES: NC(=O)c1cccc(NC(=O)c2nc3ccccc3nc2N2CCCC(F)(F)CC2)c1]

The procedure was the same as in step a-c of Example 1, except that pyridine-3-amine was replaced with 3-aminobenzamide, and after the reaction, it was extracted with ethyl acetate to obtain white solid N-(3-carbamoylphenyl)-3-(4,4-difluoroazepan-1-yl)quinoxaline-2-carboxamide (50 mg, yield 12.1%). ¹H NMR (400 MHz, CDCl₃) δ 11.10 (s, 1H), 8.24 (s, 1H), 8.02 (brs, 1H), 7.94 (dd, J=16.2, 8.1 Hz, 2H), 7.73 (d, J=4.0 Hz, 2H), 7.66 (d, J=7.8 Hz, 1H), 7.51-7.48 (m, 2H), 7.42 (br s, 1H), 3.89-3.76 (m, 2H), 3.71 (t, J=5.8 Hz, 2H), 2.41-2.38 (m, 2H), 2.13-2.04 (m, 2H), 1.95-1.92 (m, 2H). ESI-MS (m/z): 425.9 [M+H]⁺.

### Example 8

### Preparation of N-(2-carbamoylpyridin-4-yl)-2-(4,4-difluoroazepan-1-yl)quinoline-3-carboxamide (KH08)

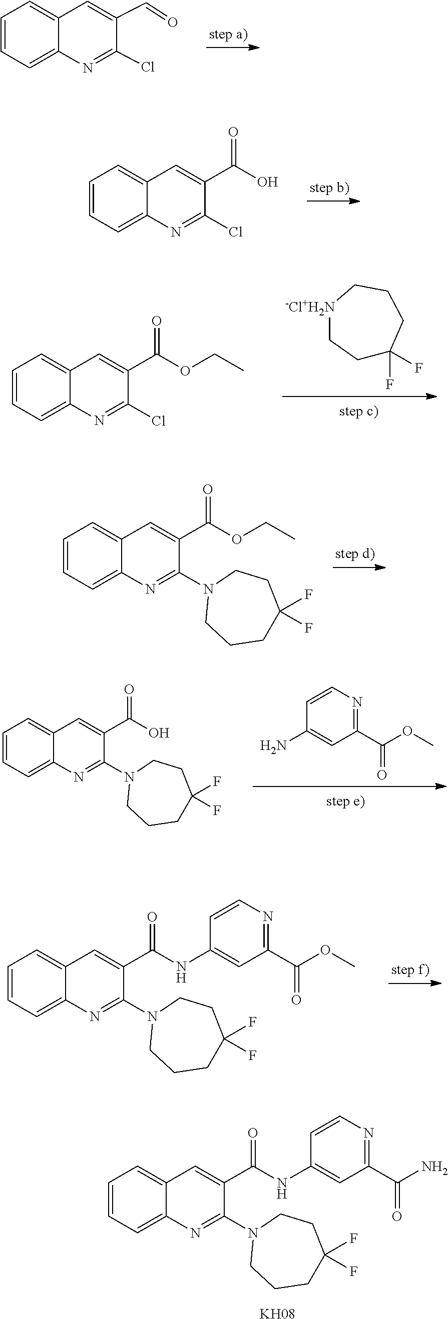

### Step a): Preparation of 2-chloroquinoline-3-carboxylic acid

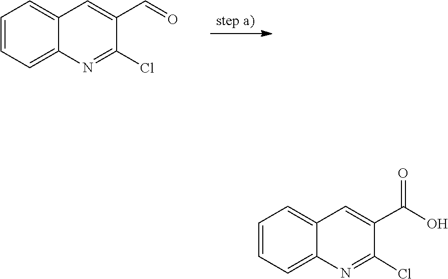 [SMILES: O=Cc1cc2ccccc2nc1Cl ; O=C(O)c1cc2ccccc2nc1Cl]

NaClO₂ (11.8 g, 130.9 mmol) and Na₂H₂PO₄ (18.5 g, 130.9 mmol) were dissolved in water (10 mL) and a solution of 2-chloroquinolin-3-formaldehyde (5 g, 26.1 mmol) in tert-butanol was added at 0° C. and stirred at room temperature for 16 h. After the reaction was completed, water was added to dilute the mixture, and the pH of the reaction mixture was adjusted to 3-4 with 1M HCl solution, and it was filtered. The residue was washed to obtain light yellow solid 2-chloroquinoline-3-carboxylic acid (2.8 g, yield 52%). ¹H NMR (400 MHz, DMSO-d₆) δ 8.86 (s, 1H), 8.16 (d, J=8.0 Hz, 1H), 8.00 (d, J=8.4 Hz, 1H), 7.92 (dd, J=11.2, 4.1 Hz, 1H), 7.72 (t, J=7.5 Hz, 1H). ESI-MS (m/z): 208.1 [M+H].⁺

### Step b): Preparation of ethyl 2-chloroquinoline-3-carboxylate

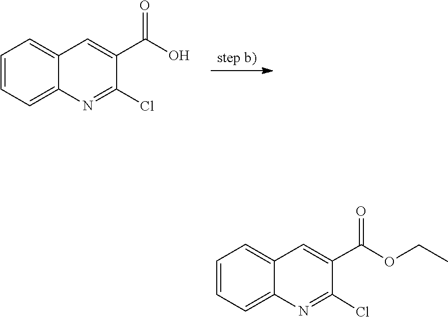 [SMILES: O=C(O)c1cc2ccccc2nc1Cl ; CCOC(=O)c1cc2ccccc2nc1Cl]

K₂CO₃ (3.7 g, 27 mmol) and ethane iodide (4.2 g, 27 mmol) were added to a solution of 2-chloroquinolin-3-carboxylic acid (2.8 g, 13.5 mmol) in DMF (20 mL) and stirred at room temperature for 16 h. After the reaction was completed, water was added to dilute the reaction mixture, and reaction system was extracted with ethyl acetate (100 mL×3). The combined organic phase was washed with saturated salt water for 3 times, and the solvent was removed by vacuum rotary evaporation. The crude product was purified by silica gel column chromatography (petroleum ether/ethyl acetate (v/v)=3/1) to obtain light yellow solid ethyl 2-chloroquinoline-3-carboxylate (2.3 g, yield 72%). ¹H NMR (400 MHz, CDCl₃) δ 8.95-8.50 (m, 1H), 8.07 (t, J=13.4 Hz, 1H), 7.90 (d, J=8.2 Hz, 1H), 7.86-7.80 (m, 1H), 7.68-7.55 (m, 1H), 4.48 (q, J=7.1 Hz, 2H), 1.46 (t, J=7.1 Hz, 3H). ESI-MS (m/z): 236.1 [M+H]⁺.

### Step c): Preparation of ethyl 2-(4,4-difluoroazepan-1-yl) quinoline-3-carboxylate

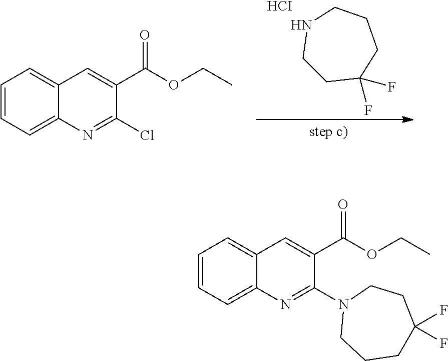 [SMILES: "HCI" ; CCOC(=O)c1cc2ccccc2nc1Cl ; FC1(F)CCCNCC1 ; CCOC(=O)c1cc2ccccc2nc1N1CCCC(F)(F)CC1]

4-Difluoroazepane hydrochloride (1.9 g, 11.64 mmol) and DIEA (3.75 g, 29.1 mmol) were added to a solution of 2-chloroquinolin-3-carboxylic acid (2.3 g, 9.7 mmol) in NMP (15 mL), respectively. And the reaction mixture was stirred at 140° C. for 16 h. After the reaction was completed, it was cooled to room temperature, and diluted with 100 mL water, then the reaction mixture was extracted with ethyl acetate (100 mL×3), and the combined organic phase was washed with saturated salt water, dried with anhydrous sodium sulfate, and filtered. The filtrate was concentrated under reduced pressure. The crude product was purified by silica gel column chromatography (petroleum ether/ethyl acetate (v/v)=5/1) to obtain light yellow oil ethyl 2-(4,4-difluoroazepan-1-yl)quinoline-3-carboxylate (2 g, yield 61%). ¹H NMR (400 MHz, CDCl₃) δ 8.31 (s, 1H), 7.68 (dd, J=10.6, 8.7 Hz, 2H), 7.60-7.56 (m, 1H), 7.25-7.21 (m, 1H), 4.40 (q, J=7.1 Hz, 2H), 3.88-3.74 (m, 2H), 3.48 (2H, t, J=5.6 Hz), 2.58-2.40 (m, 2H), 1.98 (dd, J=8.7, 4.1 Hz, 4H), 1.42 (t, J=7.1 Hz, 3H). ESI-MS (m/z): 335.0 [M+H].⁺

### Step d): Preparation of 2-(4,4-difluoroazepan-1-yl)quinoline-3-carboxylic acid

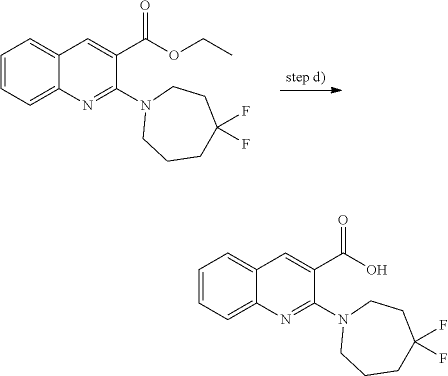 [SMILES: CCOC(=O)c1cc2ccccc2nc1N1CCCC(F)(F)CC1 ; O=C(O)c1cc2ccccc2nc1N1CCCC(F)(F)CC1]

Lithium hydroxide (288 mg, 12 mmol) was added to a solution of 2-(4,4-difluoroazepan-1-yl)quinoline-3-ethylcarboxylate (2 g, 6.0 mmol) in methanol/water (10 mL/2 mL), and reacted at room temperature for 16 h. After the reaction was completed, the pH of the reaction system was adjusted to 3-4 with 1M HCl solution, then it was extracted with ethyl acetate (50 mL×3), and the combined organic phase was washed with saturated salt water (50 mL), dried with anhydrous sodium sulfate, and filtered. The filtrate was concentrated under reduced pressure into yellow solid 2-(4,4-difluoroazepan-1-yl) quinoline-3-carboxylic acid (1.4 g, yield 70%), which was directly used in the next step. ESI-MS (⁺m/z): 306.9 [M+H].

### Step e): Preparation of N-(2-methylformate pyridin-4-yl)-2-(4,4-difluoroazepan-1-yl) quinoline-3-carboxamide

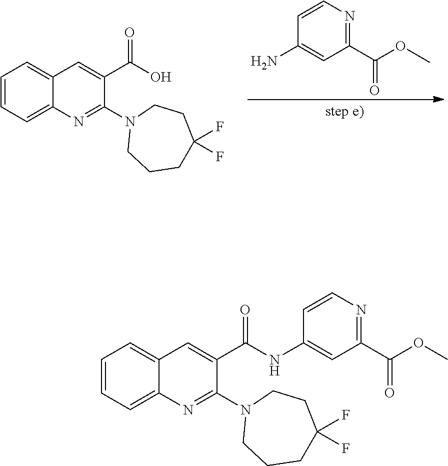 [SMILES: O=C(O)c1cc2ccccc2nc1N1CCCC(F)(F)CC1 ; COC(=O)c1cc(N)ccn1 ; COC(=O)c1cc(NC(=O)c2cc3ccccc3nc2N2CCCC(F)(F)CC2)ccn1]

Oxalyl chloride (621 mg, 4.89 mmol) and a drop of DMF were added to 2-(4,4-difluoroazepan-1-yl)quinoline-3-carboxylic acid (500 mg, 1.63 mmol) at 0° C. and reacted at room temperature for 1 h. The reaction mixture was concentrated under reduced pressure, and the obtained crude product was dissolved in 4 mL dichloromethane. Then triethylamine (823 mg, 8.15 mmol) and methyl 4-aminopyridine-2-carboxylate (300 mg, 1.95 mmol) were added, and reacted at room temperature for 16 h. After the reaction was completed, it was diluted with water and then extracted with ethyl acetate (30 mL×3). The combined organic phase was washed with saturated salt water (50 mL), dried with anhydrous sodium sulfate and filtered. Filtrate was concentrated under reduced pressure to obtain yellow solid N-(2-methylformate pyridin-4-yl)-2-(4,4-difluoroazepan-1-yl) quinoline-3-carboxamide (300 mg, yield 41%), which was directly used for the next step. ESI-MS (m/z): 441.2 [M+H]⁺.

### Step f): Preparation of N-(2-carbamoylpyridin-4-yl)-2-(4,4-difluoroazepan-1-yl)quinoline-3-carboxamide (KH08)

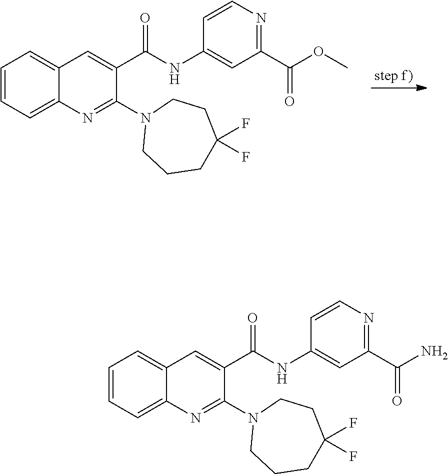 [SMILES: COC(=O)c1cc(NC(=O)c2cc3ccccc3nc2N2CCCC(F)(F)CC2)ccn1 ; NC(=O)c1cc(NC(=O)c2cc3ccccc3nc2N2CCCC(F)(F)CC2)ccn1]

N-(2-methylformate pyridin-4-yl)-2-(4,4-difluoroazepan-1-yl)quinoline-3-carboxamide (300 mg, 0.68 mmol) was dissolved in 3 mL acetonitrile. Ammonium chloride (6.3 mg, 0.12 mmol) and 1.8 mL ammonia water were added and the resulting mixture was reacted at room temperature for 3 h. After the reaction was completed, the reaction system was concentrated and the crude product was purified by Prep-HPLC to obtain white solid N-(2-carbamoylpyridine-4-yl)-2-(4,4-difluoroazepan-1-yl)quinoline-3-carboxamide (50 mg, yield 17.3%). ¹H NMR (400 MHz, DMSO-d₆) δ 11.20 (s, 1H), 8.56 (d, J=5.5 Hz, 1H), 8.48-8.33 (m, 2H), 8.11 (brs, 1H), 7.95-7.80 (m, 2H), 7.65 (d, J=3.6 Hz, 3H), 7.32-7.30 (m, 1H), 3.80-3.67 (m, 2H), 3.57 (2H, t, J=5.8 Hz), 2.43-2.31 (m, 2H), 2.01-1.98 (m, 2H), 1.89 (d, J=5.4 Hz, 2H). ESI-MS (m/z): 425.9 [M+H]⁺.

### Example 9

### Preparation of N-(3-carbamoyl-4-fluorophenyl)-2-(4,4-difluoroazepan-1-yl)quinoline-3-carboxamide (KH09)

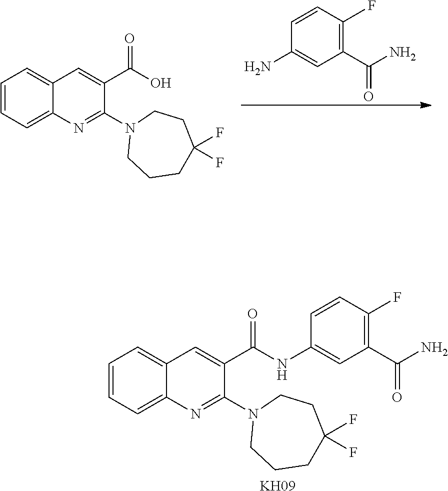

For the synthesis of 2-(4,4-difluoroazepan-1-yl)quinoline-3-carboxylic acid, see step a-d in Example 8.

HATU (296 mg, 0.778 mmol), 5-amino-2-fluorobenzamide (92.2 mg, 0.598 mmol) and DIEA (231 mg, 1.796 mmol) were added to a solution of (4,4-difluoroazepan-1-yl)quinoline-3-carboxylic acid (200 mg, 0.598 mmol) in DMF (5 mL), and reacted at room temperature for 16 h. After the reaction was completed, water was added to quench the reaction, and the organic phase was extracted with ethyl acetate (20 mL×3), washed with saturated salt water, dried with anhydrous sodium sulfate, and the filtrate was concentrated under reduced pressure. The resulting crude product was prepared by Prep-HPLC to obtain white solid powder of N-(3-carbamoyl-4-fluorophenyl)-2-(4,4-difluoroazepan-1-yl)quinoline-3-carboxamide (108.7 mg, yield 41%). ¹H NMR (400 MHz, DMSO-d₆) δ 10.79 (s, 1H), 8.32 (s, 1H), 8.03 (dd, J=6.4, 2.7 Hz, 1H), 7.86 (d, J=4.4 Hz, 1H), 7.84 (d, J=3.6 Hz, 1H), 7.68 (br s, 2H), 7.64 (br s, 2H), 7.30 (dd, J=11.9, 7.2 Hz, 2H), 3.76 (d, J=4.8 Hz, 2H), 3.62 (t, J=5.8 Hz, 2H), 2.40 (brs, 2H), 2.00 (brs, 2H), 1.89 (d, J=4.9 Hz, 2H). ESI-MS (m/z): 443.2 [M+H]⁺.

### Example 10

### Preparation of N-(2-carbamoylpyridin-4-yl)-2-(4,4-difluoroazepan-1-yl)-6,7-difluoroquinoline-3-carboxamide (KH10)

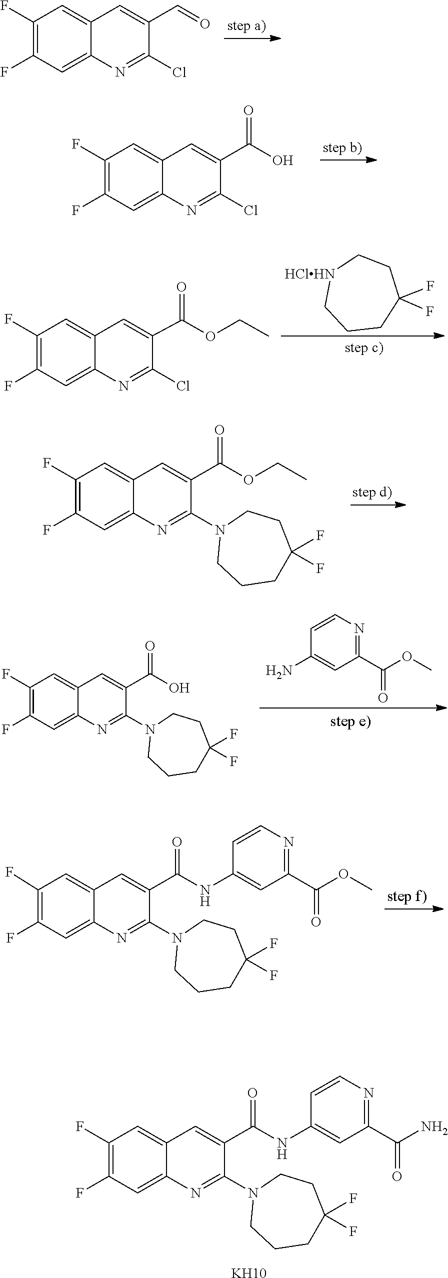

### Step a): Preparation of 2-chloro-6,7-difluoroquinolin-3-carboxylic acid

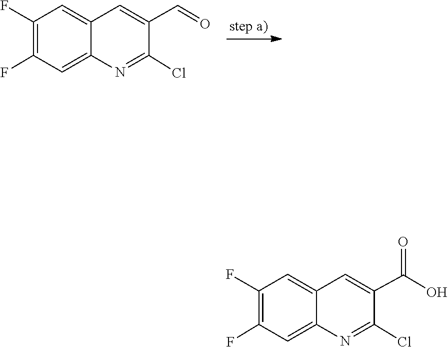 [SMILES: O=Cc1cc2cc(F)c(F)cc2nc1Cl ; O=C(O)c1cc2cc(F)c(F)cc2nc1Cl]

Silver nitrate (1.2 g, 7.02 mmol) was dissolved in a pre-configured solution of NaOH (0.88 g, 22 mmol) in ethanol/water (12 mL/3 mL) under an ice water bath, and the solution of 2-chloro-6,7-difluoroquinolin-3-carbaldehyde (1 g, 4.39 mmol) in ethanol (10 mL) was slowly dripped into it at room temperature and stirred for 16 h. After the reaction was completed, the reaction mixture was filtered, and the pH of the filtrate was adjusted to 6 with 1 M hydrochloric acid, then organic phase was extracted with ethyl acetate (50 mL×3), washed with saturated salt water (30 mL×2), dried with anhydrous sodium sulfate, and filtered, and the filtrate was concentrated under reduced pressure. The white solid 2-chloro-6,7-difluoroquinolin-3-carboxylic acid was obtained (1.0 g, yield 93.46%).

ESI-MS (m/z): 244.0 [M+H]⁺.

### Step b): Preparation of ethyl 2-chloro-6,7-difluoroquinolin-3-carboxylate

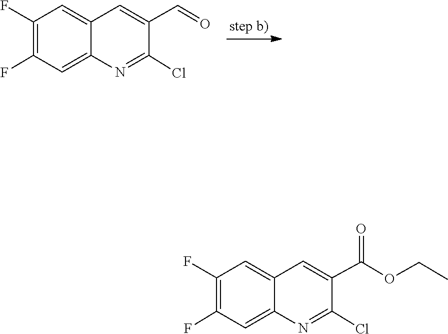 [SMILES: O=Cc1cc2cc(F)c(F)cc2nc1Cl ; CCOC(=O)c1cc2cc(F)c(F)cc2nc1Cl]

2-chloro-6,7-difluoroquinolin-3-carboxylic acid (1.0 g, 4.10 mmol) was dissolved in DMF (20 mL), then ethane iodide (1.28 g, 8.21 mmol) and potassium carbonate (1.13 g, 8.21 mmol) were added and stirred at room temperature for 16 h. Water (100 mL) was added to dilute the reaction mixture, which was extracted with ethyl acetate (50 mL×3), and the combined organic phase was washed with saturated salt water (50 mL×2), dried with anhydrous sodium sulfate, and filtered. The filtrate was concentrated under reduced pressure, and the crude product was purified by normal silica gel column (petroleum ether/ethyl acetate (v/v)=0%˜30%) to obtain white solid ethyl 2-chloro-6,7-difluoroquinolin-3-carboxylate (1.0 g, yield 89.29%).

ESI-MS (m/z): 281.2 [M+H]⁺.

### Step c): Preparation of ethyl 2-(4,4-difluoroazepan-1-yl)-6,7-difluoroquinolin-3-carboxylate

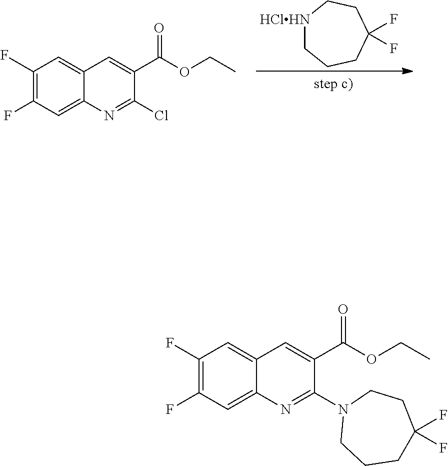

4-Difluoroazepane hydrochloride (0.81 g, 4.79 mmol) and DIEA (1.42 g, 11.04 mmol) were added to a solution of ethyl 2-chloro-6,7-difluoroquinolin-3-carboxylate (1.0 g, 3.68 mmol) in NMP (13 mL), and stirred at 140° C. for 16 h. After the reaction was completed, the reaction system was diluted with water (30 mL), extracted with ethyl acetate (20 mL×3), and the combined organic phase was washed with saturated salt water, dried with anhydrous sodium sulfate, and filtered, and the filtrate was concentrated under reduced pressure. The crude product was purified by normal silica gel column (petroleum ether/ethyl acetate (v/v)=0%˜25%) to obtain yellow oil ethyl 2-(4,4-difluoroazepan-1-yl)-6,7-difluoroquinolin-3-carboxylate (900 mg, yield 65.93%). ¹H NMR (400 MHz, CDCl₃) delta 8.21 (s, 1H), 7.49-7.32 (m, 2H), 4.40 (q, J=7.1 Hz, 2H), 3.83-3.74 (m, 2H), 3.45 (t, J=5.6 Hz, 2H), 2.53-2.37 (m, 2H), 2.04-1.92 (m, 4H), 1.42 (t, J=7.1 Hz, 3H). ESI-MS (m/z): 371.2 [M+H]⁺.

### Step d): Preparation of 2-(4,4-difluoroazepan-1-yl)-6,7-difluoroquinolin-3-carboxylic acid

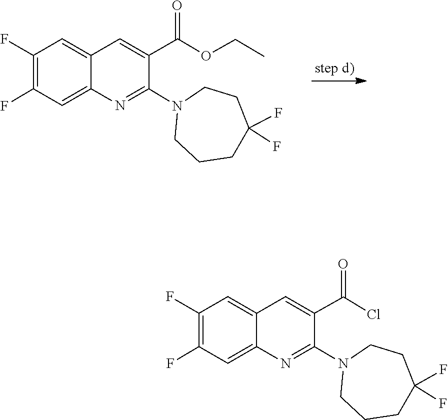 [SMILES: CCOC(=O)c1cc2cc(F)c(F)cc2nc1N1CCCC(F)(F)CC1 ; O=C(Cl)c1cc2cc(F)c(F)cc2nc1N1CCCC(F)(F)CC1]

A mixture of methanol (10 mL), water (10 mL) and THF (10 mL) was first prepared, and ethyl 2-(4,4-difluoroazepan-1-yl)-6,7-difluoroquinolin-3-carboxylate (900 mg, 2.43 mmol) was added to the prepared mixture. Then lithium hydroxide (612.36 mg, 14.58 mmol) was added and stirred at 50° C. for 3 h. The pH of reaction mixture was adjusted to 6 with 1 M hydrochloric acid, and it was extracted with ethyl acetate (15 mL×3), then the combined organic phase was washed with saturated salt water (20 mL×2), dried with anhydrous sodium sulfate, and filtered. The filtrate was concentrated under reduced pressure to obtain light yellow solid 2-(4,4-difluoroazepan-1-yl)-6,7-difluoroquinolin-3-carboxylic acid (702.03 mg, yield 84.38%). ¹H NMR (400 MHz, DMSO-d₆) δ 13.42 (brs, 1H), 8.44 (s, 1H), 7.93 (dd, J=10.9, 9.2 Hz, 1H), 7.53 (dd, J=12.3, 7.6 Hz, 1H), 3.75-3.67 (m, 2H), 3.49 (2H, t, J=5.8 Hz), 2.46-2.33 (m, 2H), 2.08-1.86 (m, 4H). ESI-MS (m/z): 343.2 [M+H]⁺.

### Step e): Preparation of methyl 4-(2-(4,4-difluoroazepan-1-yl)-6,7-difluoroquinoline-3-carboxamido)pyridine carboxylate

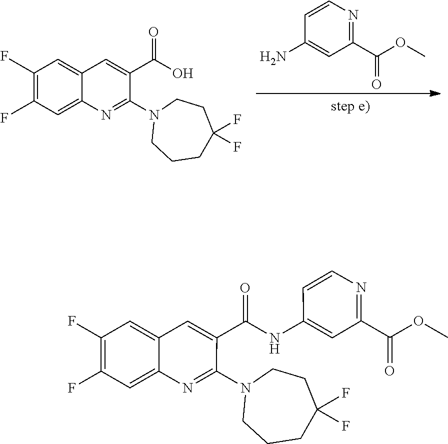 [SMILES: O=C(O)c1cc2cc(F)c(F)cc2nc1N1CCCC(F)(F)CC1 ; COC(=O)c1cc(N)ccn1 ; COC(=O)c1cc(NC(=O)c2cc3cc(F)c(F)cc3nc2N2CCCC(F)(F)CC2)ccn1]

At room temperature, 2-(4,4-difluoroazepan-1-yl)-6,7-difluoroquinolin-3-carboxylic acid (300 mg, 0.88 mmol) was dissolved in dichloromethane (10 mL), then DMF (0.05 mL) was added, and oxalyl chloride (335.09 mg, 2.64 mmol) was slowly added. The resulting mixture was stirred at room temperature for 1 h. The solvent was removed by vacuum rotary evaporation and the resulting residue was dissolved in dichloromethane (5 mL). Then methyl 4-aminopyridine-2-carboxylate (401.68 mg, 2.64 mmol) and triethylamine (445.23 mg, 4.40 mmol) were added. The reaction mixture was stirred at room temperature for 16 h. After the reaction was completed, the reaction system was quenched with saturated sodium bicarbonate solution, extracted with dichloromethane (30 mL×3), and the combined organic phase was washed with saturated salt water, dried with anhydrous sodium sulfate, and filtered, and the filtrate was concentrated under reduced pressure. The resulting crude product was purified by normal silica gel column (petroleum ether/ethyl acetate (v/v)=0%˜35%) to obtain light yellow solid methyl 4-(2-(4,4-difluoroazepan-1-yl)-6,7-difluoroquinoline-3-carboxamido)pyridine carboxylate (152 mg, yield 36.4%). ESI-MS (m/z): 476.8 [M+H]⁺.

### Step f): Preparation of N-(2-carbamoylpyridine-4-yl)-2-(4,4-difluoroazepan-1-yl)-6,7-difluoroquinoline-3-carboxamide (KH10)

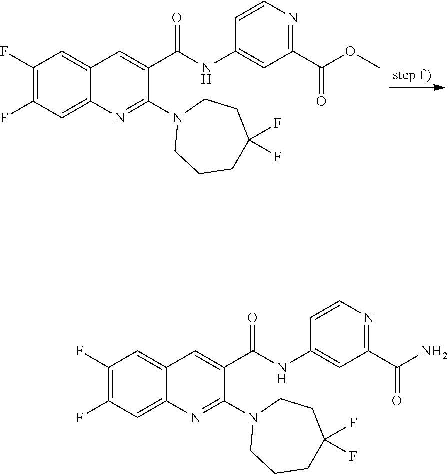 [SMILES: COC(=O)c1cc(NC(=O)c2cc3cc(F)c(F)cc3nc2N2CCCC(F)(F)CC2)ccn1 ; NC(=O)c1cc(NC(=O)c2cc3cc(F)c(F)cc3nc2N2CCCC(F)(F)CC2)ccn1]

Ammonium chloride solid (5 mg, 0.09 mmol) and ammonia water (5 mL) were added to a solution of methyl 4-(2-(4,4-difluoroazepan-1-yl)-6,7-difluoroquinolin-3-carboxamido)pyridine carboxylate (150 mg, 0.315 mmol) in acetonitrile (5 mL) and react at 40° C. for 4 h, and water (20 mL) was added to dilute the reaction solution. The organic phase was extracted with ethyl acetate (35 mL×3), washed with saturated salt water, dried with anhydrous sodium sulfate, and filtered. The filtrate was concentrated under reduced pressure. The resulting crude product was purified by Prep-HPLC to obtain white solid N-(2-carbamoylpyridine-4-yl)-2-(4,4-difluoroazepan-1-yl)-6,7-difluoroquinoline-3-carboxamide (53.77 mg, yield 37.0%). ¹H NMR (400 MHz, DMSO-d₆) δ 11.26 (s, 1H), 8.56 (d, J=5.5 Hz, 1H), 8.45-8.32 (m, 2H), 8.11 (d, J=2.2 Hz, 1H), 7.97-7.85 (m, 2H), 7.66 (d, J=2.2 Hz, 1H), 7.60 (dd, J=12.2, 7.6 Hz, 1H), 3.74-3.71 (m, 2H), 3.55 (t, J=5.8 Hz, 2H), 2.39-2.38 (m, 2H), 1.97-1.95 (m, 2H), 1.88-1.86 (m, 2H). ESI-MS (m/z): 462.3 [M+H]⁺.

### Example 11

### Preparation of N-(3-carbamoyl-4-fluorophenyl)-3-(4,4-difluoroazepan-1-yl) quinoline-2-carboxamide (KH11)

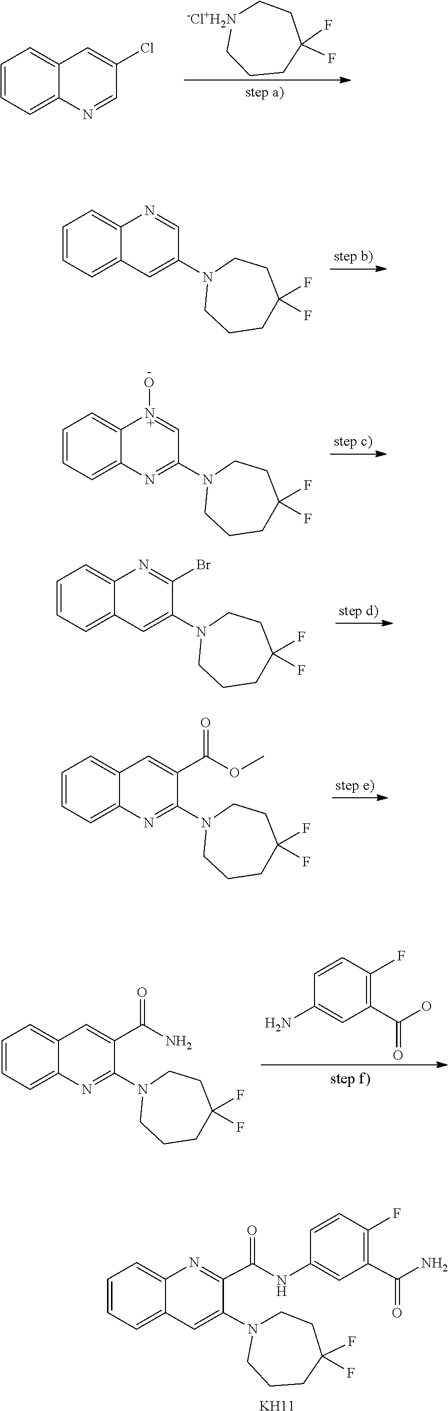

### Step a): Preparation of 3-(4,4-difluoroazepan-1-yl) quinoline

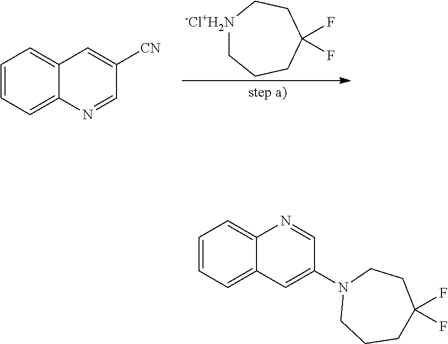

4,4-Difluoroazepane hydrochloride (4.49 g, 26.4 mmol), Ruphos-Pd-G2 (0.513 g, 0.66 mmol) and potassium tert-butanol (7.4 g, 66 mmol) were added to a solution of 3-chloroquinoline (3.6 g, 22 mmol) in 1,4-dioxane (30 mL) and stirred at 105° C. for 16 h under the protection of nitrogen. After the reaction was completed, the reaction system was quenched with water (100 mL), extracted with ethyl acetate (50 mL×3), and the combined organic phase was washed with saturated salt water (50 mL×3), dried with anhydrous sodium sulfate, and filtered, and the filtrate was concentrated under reduced pressure. The crude product was purified by silica gel column chromatography (ethyl acetate/petroleum ether (v/v)=1/20) to obtain yellow solid 3-(4,4-difluoroazepan-1-yl) quinoline (3 g, yield 51.7%). ¹H NMR (400 MHz, DMSO-d₆) δ 8.73 (d, J=2.5 Hz, 1H), 7.82 (d, J=8.1 Hz, 1H), 7.72 (d, J=7.9 Hz, 1H), 7.45-7.28 (m, 3H), 3.68 (d, J=4.9 Hz, 2H), 3.60 (2H, t, J=5.8 Hz), 2.39-2.24 (m, 2H), 2.17-2.02 (m, 2H), 1.97-1.85 (m, 2H). ESI-MS (m/z): 263.2 [M+H]⁺.

### Step b): Preparation of 3-(4,4-difluoroazepan-1-yl) quinoline-oxide

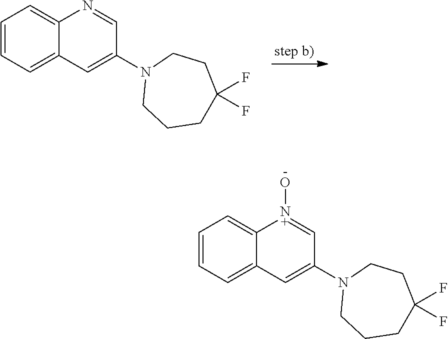

m-Chloroperoxybenzoic acid (3.4 g, 19.7 mmol) was slowly added to the solution of 3-(4,4-difluoroazepan-1-yl)quinoline (2.6 g, 9.88 mmol) in dichloromethane (40 mL) and reacted at 40° C. for 48 h. After the reaction was completed, the reaction system was quenched with water (50 mL), extracted with ethyl acetate (50 mL×3), and the combined organic phase was washed twice with 10% sodium thiosulfate aqueous solution, and then dried with anhydrous sodium sulfate, filtered and the filtrate was concentrated under reduced pressure. The crude product was purified by silica gel column chromatography (ethyl acetate/petroleum ether (v/v)=1/5) to obtain white solid 3-(4,4-difluoroazepan-1-l) quinoline-1-oxide (1.5 g, yield 55.5%). ESI-MS (m/z): 279.3 [M+H]⁺.

### Step c): Preparation of 2-bromo-3-(4,4-difluoroazepan-1-yl) quinoline

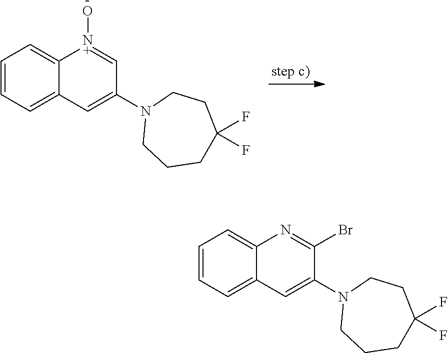

N-bromosuccinimide (1.9 g, 10.7 mmol) and triphenylphosphine (2.83 g, 10.7 mmol) were added to a solution of 3-(4,4-difluoroazepan-1-yl) quinoline-1-oxide (1.5 g, 5.37 mmol) in toluene (20 mL) and reacted at 115° C. for 2 h. After the reaction was completed, the reaction system was quenched with water (40 mL), extracted with ethyl acetate (30 mL×3), and the combined organic phase was washed with saturated salt water (30 mL×3), dried with anhydrous sodium sulfate, filtered, and the filtrate was concentrated under reduced pressure. The crude product was purified by silica gel column chromatography (ethyl acetate/petroleum ether (v/v)=1/7) to obtain white solid 2-bromo-3-(4,4-difluoroazepan-1-l) quinoline (100 mg, yield 5.4%). ¹H NMR (400 MHz, DMSO-d₆) delta 7.95-7.85 (m, 2H), 7.61-7.55 (m, 3H), 3.29 (br s, 4H), 2.36-2.30 (m, 4H), 1.86 (br s, 2H). ESI-MS (m/z): 341.2 [M+H]⁺.

### Step d): Preparation of methyl 3-(4,4-difluoroazepan-1-yl) quinoline-2-carboxylate

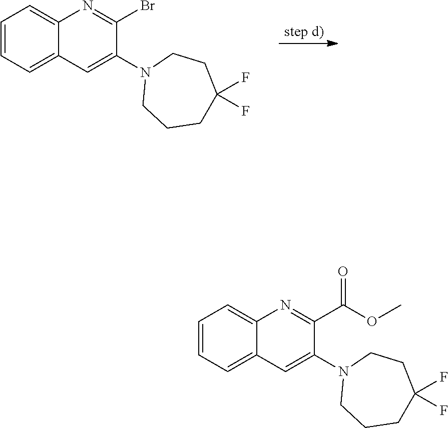 [SMILES: FC1(F)CCCN(c2cc3ccccc3nc2Br)CC1 ; COC(=O)c1nc2ccccc2cc1N1CCCC(F)(F)CC1]

Triethylamine (88.8 mg, 0.879 mmol) and Pd(dppf)Cl₂ (23 mg, 0.0293 mmol) were added to a solution of 2-bromo-3-(4,4-difluoroazepan-1-yl)quinoline (100 mg, 0.293 mmol) in methanol (20 mL), and replaced gas with carbon monoxide three times, and the reaction system was reacted at 110° C. in an autoclave (4 atm) for 16 hours. After the reaction was completed, the reaction system was quenched with water (40 mL), extracted with ethyl acetate (20 mL×3), and the combined organic phase was washed with saturated salt water (30 mL), dried with anhydrous sodium sulfate, filtered, and the filtrate was concentrated under reduced pressure. The crude product was purified by silica gel column chromatography (ethyl acetate/petroleum ether (v/v)=1/5) to obtain yellow solid methyl 3-(4,4-difluoroazepan-1-yl) quinoline-2-carboxylate (55 mg, yield 58%). ¹H NMR (400 MHz, DMSO-d₆) δ 7.98-7.84 (m, 3H), 7.63-7.45 (m, 2H), 3.939 (s, 3H), 3.40-3.34 (m, 4H), 2.33-2.23 (m, 2H), 2.21-2.14 (m, 2H), 1.91-1.83 (m, 2H). ESI-MS (m/z): 321.3 [M+H]⁺.

### Step e): Preparation of 3-(4,4-difluoroazepan-1-yl) quinoline-2-carboxylic acid

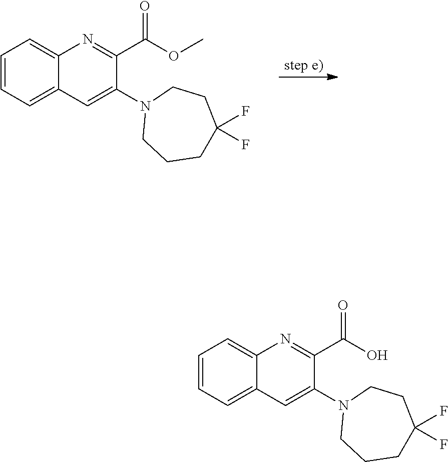 [SMILES: COC(=O)c1nc2ccccc2cc1N1CCCC(F)(F)CC1 ; O=C(O)c1nc2ccccc2cc1N1CCCC(F)(F)CC1]

Lithium hydroxide (25 mg, 0.601 mmol) was slowly added to a mixture of methyl 3-(4,4-difluoroazepan-1-yl) quinoline-2-carboxylate (55 mg, 0.178 mmol) in water (0.5 mL) and tetrahydrofuran (2 mL), and reacted at room temperature for 1 h. After the reaction was completed, the system was quenched with ice water, and 1 M of diluted hydrochloric acid was added to adjust the pH to 7. Then it was extracted with ethyl acetate (10 mL×3), and the combined organic phase was washed with saturated salt water, dried with anhydrous sodium sulfate, filtered, and the filtrate was concentrated under reduced pressure to obtain yellow solid 3-(4,4-difluoroazepan-1-yl)quinoline-2-carboxylic acid (40 mg, yield 76%). ESI-MS (m/z): 307.1 [M+H]⁺.

### Step f): Preparation of N-(3-carbamoyl-4-fluorophenyl)-3-(4,4-difluoroazepan-1-yl) quinoline-2-carboxamide (KH11)

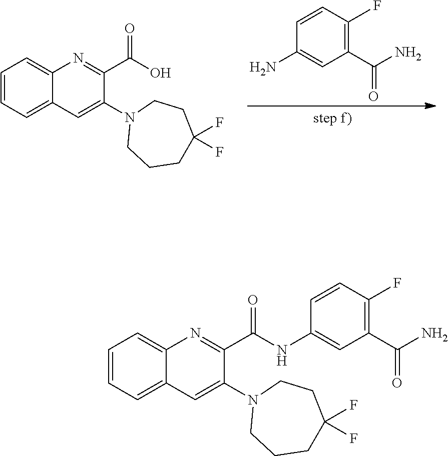 [SMILES: O=C(O)c1nc2ccccc2cc1N1CCCC(F)(F)CC1 ; NC(=O)c1cc(N)ccc1F ; NC(=O)c1cc(NC(=O)c2nc3ccccc3cc2N2CCCC(F)(F)CC2)ccc1F]

HATU (60 mg, 0.1302 mmol), 5-amino-2-fluorobenzamide (20 mg, 0.1302 mmol) and triethylamine (39 mg, 0.3908 mmol) were added to a solution of 3-(4,4-difluoroazepan-1-yl) quinoline-2-carboxylic acid (40 mg, 0.1302 mmol) in dichloromethane (2 mL) and reacted at room temperature for 2 h. After the reaction was completed, the reaction system was quenched with water (20 mL), extracted with ethyl acetate (10 mL×3), and the combined organic phase was washed with saturated salt water (10 mL), dried with anhydrous sodium sulfate, filtered, and the filtrate was concentrated under reduced pressure. The crude product was purified by Prep-HPLC to obtain a solid powder N-(3-carbamoyl-4-fluorophenyl)-3-(4,4-difluoroazepan-1-yl) quinoline-2-carboxamide (3.10 mg, yield 5.3%). ¹H NMR (400 MHz, MeOD) δ 8.16 (dd, J=6.4, 2.7 Hz, 1H), 8.05-7.97 (m, 2H), 7.96 (s, 1H), 7.86 (d, J=8.3 Hz, 1H), 7.65-7.55 (m, 2H), 7.32-7.24 (m, 1H), 3.49-3.43 (m, 4H), 2.35-2.24 (m, 2H), 2.17-2.01 (m, 2H), 1.98-1.90 (m, 2H). ESI-MS (m/z): 443.2 [M+H]⁺.

### Example 12

### Preparation of N-(3-carbamoylphenyl)-2-(4,4-difluoroazepan-1-yl)-6,7-difluoroquinoline-3-carboxamide (KH12)

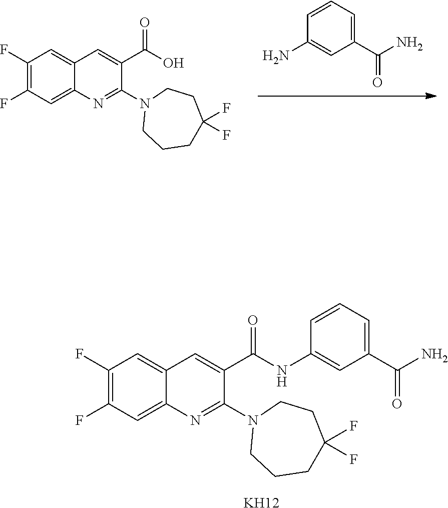

2-(4,4-difluoroazepan-1-yl)-6,7-difluoroquinolin-3-carboxylic acid was prepared the same as in step a-d of example 10.

(4,4-Difluoroazepan-1-yl)-6,7-difluoroquinolin-3-carboxylic acid (100 mg, 0.29 mmol) was dissolved in DMF (2 mL), then 3-aminobenzoamide (43.75 mg, 0.32 mmol), HATU (113.3 mg, 0.35 mmol) and DIEA (113.27 mg, 0.87 mmol) were added and stirred at room temperature for 4 h. Water (20 mL) was added to dilute the reaction system, then it was extracted with ethyl acetate (10 ml×3), washed with saturated salt water (10 ml×2), and the organic phase was dried with anhydrous sodium sulfate and concentrated. White solid N-(3-carbamoylphenyl)-2-(4,4-difluoroazepan-1-yl)-6,7-difluoroquinoline-3-carboxamide was prepared by Prep-HPLC (3.2 mg, yield 2.37%). ¹H NMR (400 MHz, DMSO-d₆) δ 10.80 (s, 1H), 8.31 (s, 1H), 8.19 (s, 1H), 8.01-7.88 (m, 2H), 7.85 (d, J=8.0 Hz, 1H), 7.62 (d, J=7.8 Hz, 2H), 7.43-7.40 (m, 2H), 3.82-3.67 (m, 2H), 3.61 (2H, t, J=5.9 Hz), 2.36-2.32 (m, 2H), 2.00 (brs, 2H), 1.89 (brs, 2H). ESI-MS (m/z): 461.3 [M+H]⁺.

### Example 13

### Preparation of 2-(4,4-difluoroazepan-1-yl)-N-(3-sulfamoylphenyl)quinoline-3-carboxamide (KH13)

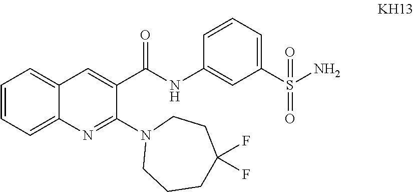 [SMILES: NS(=O)(=O)c1cccc(NC(=O)c2cc3ccccc3nc2N2CCCC(F)(F)CC2)c1]

The preparation of 3-(4,4-difluoroazepan-1-yl)-N-(3-sulfamoylphenyl)quinoline-3-carboxamide is similar to step a-d of example 8 and example 6, except that the 3-(4,4-difluoroazepan-1-yl)quinoline-2-carboxylic acid in step f of Example 6 is substituted with the 2-(4,4-difluoroazepan-1-yl)quinoline-3-carboxylic acid synthesized through steps a-d of Example 8, and the 4-bromo-2-fluoropyridine in step a of Example 6 is replaced with 4-bromo-2-fluorobenzene, and change the room temperature of step e in Example 6 to 80° C., resulting in a white solid 2-(4,4-difluoroazepan-1-yl)-N-(3-sulfamoylphenyl)quinoline-3-carboxamide (22.12 mg, yield 30.56%). ¹H NMR (400 MHz, DMSO-d6) δ 10.96 (s, 1H), 8.33 (s, 2H), 7.93-7.75 (m, 2H), 7.64 (d, J=3.6 Hz, 2H), 7.57 (dd, J=8.0, 5.5 Hz, 2H), 7.42 (s, 2H), 7.35-7.25 (m, 1H), 3.83-3.70 (m, 2H), 3.61 (t, J=5.9 Hz, 2H), 2.43-2.40 (m, 2H), 2.00-1.95 (m, 2H), 1.90 (br s, 2H). ESI-MS (m/z): 461.1 [M+H]⁺.

### Example 14

### Preparation of 2-(4,4-difluoroazepan-1-yl)-N-(2-sulfamoylpyridine-4-yl) quinoline-3-carboxamide (KH14)

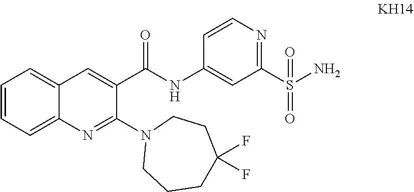 [SMILES: NS(=O)(=O)c1cc(NC(=O)c2cc3ccccc3nc2N2CCCC(F)(F)CC2)ccn1]

The preparation of 2-(4,4-difluoroazepan-1-yl)-N-(2-sulfamoylpyridine-4-yl)quinoline-3-carboxamide is similar to step a-d of Example 8 and Example 6, except that the 3-(4,4-difluoroazepan-1-yl)quinoline-2-carboxylic acid in step f of Example 6 is substituted with 2-(4,4-difluoroazepan-1-yl)quinoline-3-carboxylic acid synthesized through steps a-d of Example 8. The room temperature in step e of Example 6 is changed to 80° C., resulting in the white solid 2-(4,4-difluoroazepan-1-yl)-N-(2-sulfamoylpyridine-4-yl) quinoline-3-carboxamide (7.49 mg, yield 11.4%). ¹H NMR (400 MHz, DMSO-d₆) δ 11.34 (s, 1H), 8.63 (d, J=5.4 Hz, 1H), 8.41 (s, 1H), 8.34 (d, J=1.7 Hz, 1H), 7.90-7.81 (m, 2H), 7.65 (d, J=3.6 Hz, 2H), 7.47 (s, 2H), 7.37-7.27 (m, 1H), 3.77-3.69 (m, 2H), 3.55 (t, J=5.9 Hz, 2H), 2.43-2.40 (m, 2H), 1.98-1.95 (m, 2H), 1.89 (br s, 2H). ESI-MS (m/z): 462.1 [M+H]⁺.

### Example 15

### Preparation of N-(2-carbamoylpyridin-4-yl)-3-(4,4-difluoroazepan-1-yl) quinoline-2-carboxamide (KH15)

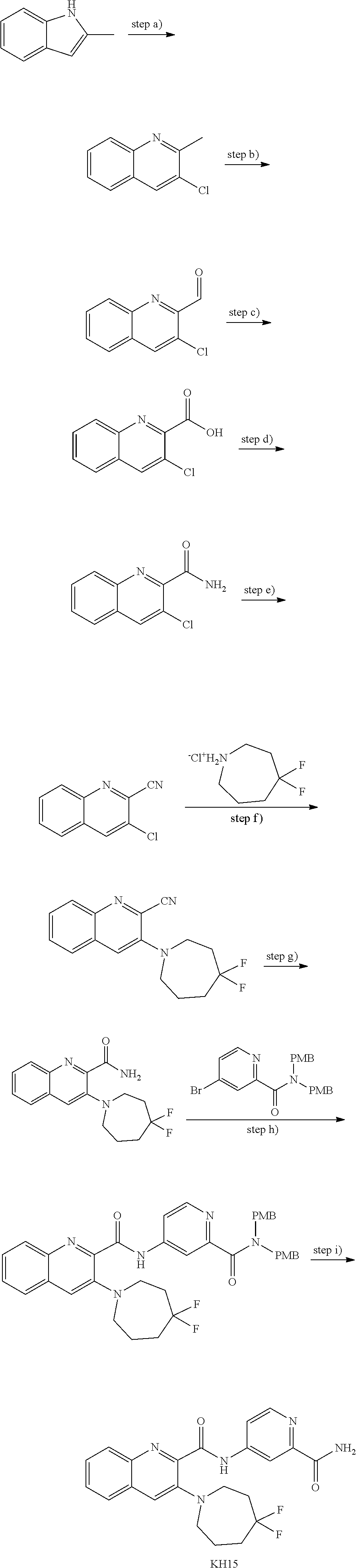

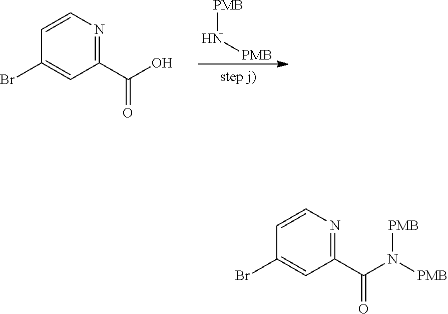

### Step a): Preparation of 3-chloro-2-methylquinoline

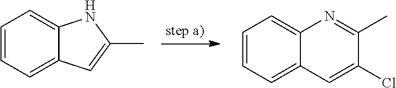

The solution of benzyltriethylammonium chloride (1.74 g, 7.63 mmol) in chloroform (300 mL) was slowly added to a solution of 2-methyl-1H-indole (10 g, 76.3 mmol) in 50% KOH (32 mL) in a 0° C. ice bath, and stirred at room temperature for 16 hours. After the reaction was completed, it was quenched with water (200 mL), extracted with dichloromethane (200 mL×3), and the combined organic phase was washed with saturated salt water (80 mL×3), dried with anhydrous sodium sulfate, filtered, and concentrated under reduced pressure. The resulting crude product was purified by silica gel column chromatography (ethyl acetate/petroleum ether (v/v)=1/20) to obtain yellow solid 3-chloro-2-methylquinoline (2.1 g, yield 15%). ¹H NMR (400 MHz, DMSO-d₆) δ 8.55 (s, 1H), 8.06-7.94 (m, 2H), 7.80-7.78 (m, 1H), 7.71-7.56 (m, 1H), 2.77 (s, 3H). ESI-MS (m/z): 178.1 [M+H]⁺.

### Step b): Preparation of 3-chloroquinoline-2-carbaldehyde

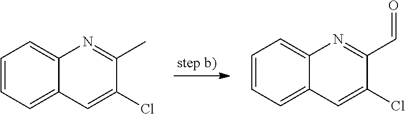

Selenium dioxide (3.76 g, 33.8 mmol) was slowly added to a solution of 3-chloro-2-methylquinoline (2 g, 11.2 mmol) in 1,4-dioxane (40 mL) and reacted at 100° C. for 2 hours. After the reaction was completed, it was cooled to room temperature, then filtered, and the filtrate was concentrated under reduced pressure to obtain a white solid 3-chloroquinoline-2-carbaldehyde (2.1 g, yield 97%). ESI-MS (m/z): 192.1 [M+H]⁺.

### Step c): Preparation of 3-chloroquinoline-2-carboxylic acid

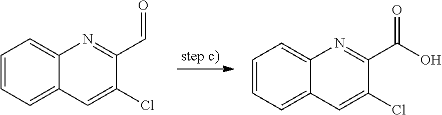 [SMILES: O=Cc1nc2ccccc2cc1Cl ; O=C(O)c1nc2ccccc2cc1Cl]

A mixed aqueous solution of sodium chlorite (4.7 g, 52.3 mmol) and disodium hydrogen phosphate (7.4 g, 52.3 mmol) were slowly added to a solution of 3-chloroquinoline-2-carbaldehyde (2 g, 10.47 mmol) in tert-butanol (20 mL) in a 0° C. ice bath (36 mL) and stirred at room temperature for 16 hours. After the reaction was completed, the pH was adjusted to neutral with 1 M hydrochloric acid aqueous solution, then the organic phase was extracted with ethyl acetate (100 mL×3), washed with saturated salt water (80 mL), dried with anhydrous sodium sulfate, filtered, and the filtrate was concentrated under reduced pressure to obtain a white solid 3-chloroquinoline-2-carboxylic acid (1.9 g, yield 88%). ¹H NMR (400 MHz, DMSO-d₆) δ 8.79 (s, 1H), 8.11 (dd, J=19.2, 8.3 Hz, 2H), 7.93 (dd, J=11.2, 4.1 Hz, 1H), 7.80 (t, J=7.5 Hz, 1H). ESI-MS (m/z): 208.1 [M+H]⁺.

### Step d): Preparation of 3-chloroquinoline-2-carboxamide

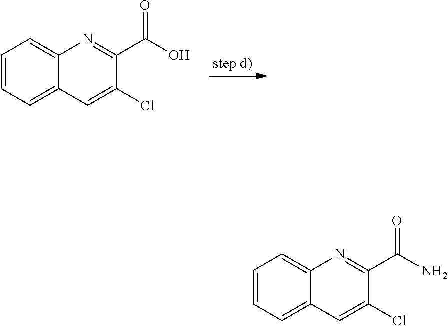 [SMILES: O=C(O)c1nc2ccccc2cc1Cl ; NC(=O)c1nc2ccccc2cc1Cl]

Ammonium chloride (231.5 mg, 4.33 mmol), HATU (657.7 mg, 1.73 mmol) and N,N-diisopropylethylamine (558 mg, 4.33 mmol) were added to a solution of 3-chloroquinolin-2-carboxylic acid (300 mg, 1.44 mmol) in DMF (3 mL) and reacted at room temperature for 4 h. After the reaction was completed, the reaction system was quenched with water (20 mL), extracted with ethyl acetate (30 mL×3), and the combined organic phase was washed with saturated salt water (30 mL×2), dried with anhydrous sodium sulfate, filtered, and concentrated under reduced pressure. The crude product was purified by silica gel column chromatography (methanol/dichloromethane (v/v)=1/10) to obtain white solid 3-chloroquinoline-2-carboxamide (180 mg, 60% yield). ¹H NMR (400 MHz, DMSO-d₆) δ 8.68 (s, 1H), 8.17 (s, 1H), 8.07 (d, J=8.5 Hz, 1H), 8.02 (d, J=8.2 Hz, 1H), 7.86 (t, J=7.4 Hz, 2H), 7.73 (t, J=7.5 Hz, 1H). ESI-MS (m/z): 207.3 [M+H].⁺

### Step e): Preparation of 3-chloroquinoline-2-carbonitrile

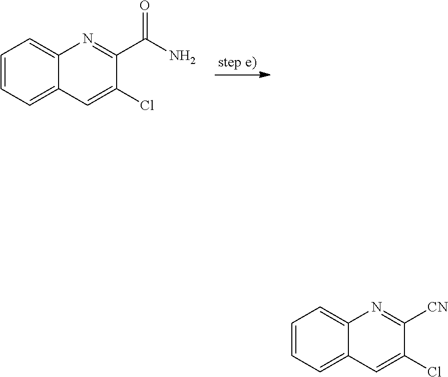 [SMILES: NC(=O)c1nc2ccccc2cc1Cl ; N#Cc1nc2ccccc2cc1Cl]

Triethylamine (220 mg, 2.17 mmol) and trifluoroacetic anhydride (220 mg, 1.04 mmol) were added to a solution of 3-chloroquinolin-2-carboxamide (180 mg, 0.87 mmol) in tetrahydrofuran (5 mL) under a 0° C. ice bath, and continually stirred under the ice bath for 1 h. After the reaction was completed, the reaction system was quenched with water (10 mL), extracted with ethyl acetate (20 mL×3), and the combined organic phase was washed with saturated salt water (20 mL×3), dried with anhydrous sodium sulfate and filtered, and the filtrate was concentrated under reduced pressure. The crude product was purified by silica gel column chromatography (ethyl acetate/petroleum ether (v/v)=1/10) to obtain yellow solid 3-chloroquinoline-2-carbonitrile (100 mg, yield 61%). ¹H NMR (400 MHz, DMSO-d₆) δ 8.93 (s, 1H), 8.16 (d, J=8.4 Hz, 1H), 8.10 (d, J=8.1 Hz, 1H), 7.97 (t, J=7.6 Hz, 1H), 7.87 (t, J=7.5 Hz, 1H). ESI-MS (m/z): 189.0 [M+H].⁺

### Step f): Preparation of 3-(4,4-difluoroazepan-1-yl) quinoline-2-carbonitrile

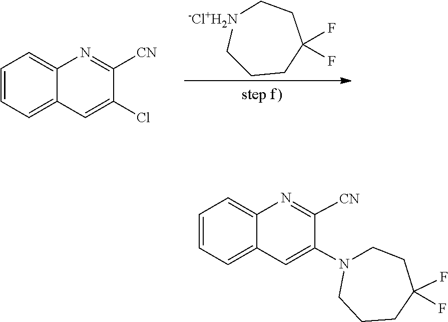

4,4-Difluoroazepane hydrochloride (400 mg, 2.34 mmol), Ruphos-Pd-G2 (140 mg, 0.179 mmol) and cesium carbonate (1750 mg, 5.4 mmol) were added to the solution of 3-chloroquinolin-2-carbonitrile (340 mg, 1.80 mmol) in toluene (8 mL) and stirred at 110° C. for 16 h under the protection of nitrogen. After the reaction was completed, the reaction system was quenched with water (20 mL), extracted with ethyl acetate (20 mL×3), and the combined organic phase was washed with saturated salt water (20 mL), dried with anhydrous sodium sulfate, filtered, and the filtrate was concentrated under reduced pressure. The crude product was purified by silica gel column chromatography (ethyl acetate/petroleum ether (v/v)=1/5) to obtain yellow oil 3-(4,4-difluoroazepan-1-yl) quinoline-2-carbonitrile (200 mg, yield 48%). ESI-MS (m/z): 228.1 [M+H].⁺

### Step g): Preparation of 3-(4,4-difluoroazepan-1-yl) quinoline-2-carboxamide

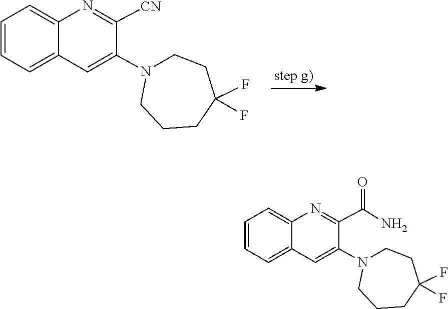 [SMILES: N#Cc1nc2ccccc2cc1N1CCCC(F)(F)CC1 ; NC(=O)c1nc2ccccc2cc1N1CCCC(F)(F)CC1]

Cesium carbonate (437 mg, 1.315 mmol) was added to a solution of 3-(4,4-difluoroazepan-1-yl)quinoline-2-carbonitrile (200 mg, 0.877 mmol) in dimethyl sulfoxide (3 mL), and 30% hydrogen peroxide (0.5 mL) was slowly added at 0° C. The reaction was carried out under ice bath for 2 h. After the reaction was completed, ice water (10 mL) was added to quench the reaction, which was extracted with ethyl acetate (10 mL×3), and the combined organic phase was washed with saturated salt water (20 mL), dried with anhydrous sodium sulfate, and filtered. Filtrate was concentrated under reduced pressure to yellow solid 3-(4,4-difluoroazepan-1-yl) quinoline-2-carboxamide (120 mg, yield 45%). ESI-MS (m/z): 306.3 [M+H].⁺

### Step h): Preparation of N-(2-(bis(4-methoxybenzyl)carbamoyl)pyridin-4-yl)-3-(4,4-difluoroazepan-1-yl)quinoline-2-carboxamide

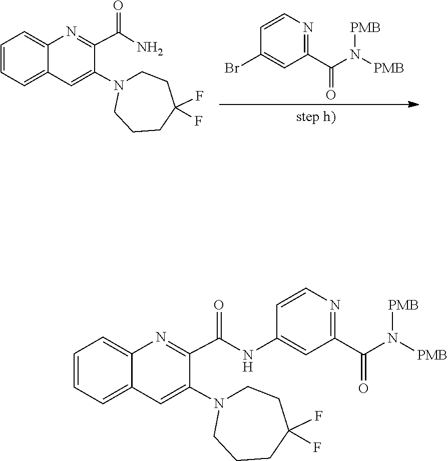

3-(4,4-Difluoroazepan-1-yl)quinoline-2-carboxamide (120 mg, 0.393 mmol), Ruphos-Pd-G2 (30.6 mg, 0.0393 mmol) and cesium carbonate (320 mg, 2.5 mmol) were added to the solution of 4-bromo-N,N-bis(4-methoxybenzyl)pyridine-2-carboxamide (208.7 mg, 0.472 mmol) in 1,4-dioxane (4 mL) and stirred at 105° C. for 16 h under nitrogen protection. After the reaction was completed, the reaction system was quenched with water (10 mL), extracted with ethyl acetate (15 mL×3), and the combined organic phase was washed with saturated salt water (10 mL×3), dried with anhydrous sodium sulfate, filtered, and the filtrate was concentrated under reduced pressure. The resulting crude product was purified by silica gel column chromatography (ethyl acetate/petroleum ether (v/v)=1/1) to obtain yellow solid N-(2-(bis(4-methoxybenzyl)carbamoyl)pyridine-4-yl)-3-(4,4-difluoroazepan-1-yl)quinoline-2-carboxamide (180 mg, yield 65%). ESI-MS (m/z): 666.5 [M+H].⁺

### Step i): Preparation of N-(2-carbamoylpyridine-4-yl)-3-(4,4-difluoroazepan-1-yl) quinoline-2-carboxamide

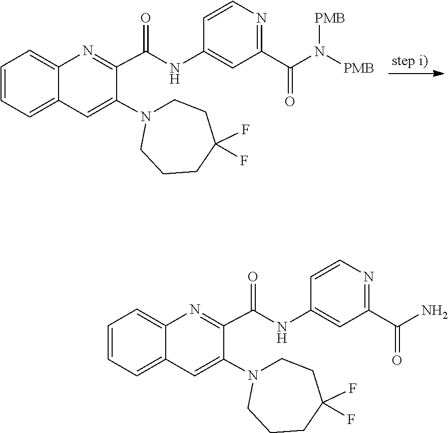

Trifluoroacetic acid solution (5 mL) and a drop of trifluoromethanesulfonic acid were added to the solution of N-(2-(bis(4-methoxybenzyl)carbamoyl)pyridine-4-yl)-3-(4,4-difluoroazepan-1-yl)quinoline-2-carboxamide (170 mg, 0.255 mmol) in 1,2-dichloroethane (3 mL) and reacted at 70° C. for 1 h. After the reaction was completed, the reaction mixture was concentrated under reduced pressure, the pH of the resulting mixture was adjusted to neutral with saturated sodium bicarbonate aqueous solution, then it was extracted with ethyl acetate (15 mL×3), and the combined organic phase was washed with saturated salt water (10 mL), dried with anhydrous sodium sulfate, filtered, and the filtrate was concentrated under reduced pressure. The resulting crude product was prepared by Prep-HPLC to yellow solid N-(2-carbamoylpyridine-4-yl)-3-(4,4-difluoroazepan-1-yl)quinoline-2-carboxamide (15 mg, yield 14%). ¹H NMR (400 MHz, DMSO-d₆) δ 11.33 (s, 1H), 8.58 (d, J=5.5 Hz, 1H), 8.44 (d, J=1.9 Hz, 1H), 8.12 (s, 1H), 8.00-7.96 (m, 1H), 7.96 (d, J=2.3 Hz, 1H), 7.95-7.93 (m, 1H), 7.92 (dd, J=4.6, 3.3 Hz, 1H), 7.67 (s, 1H), 7.64-7.57 (m, 2H), 3.43-3.38 (m, 4H), 2.26-2.23 (m, 2H), 2.13-2.09 (m, 2H), 1.90-1.81 (m, 2H). ESI-MS (m/z): 426.4 [M+H]⁺.

### Step j): Preparation of 4-bromo-N,N-bis(4-methoxybenzyl)pyridine-2-carboxamide

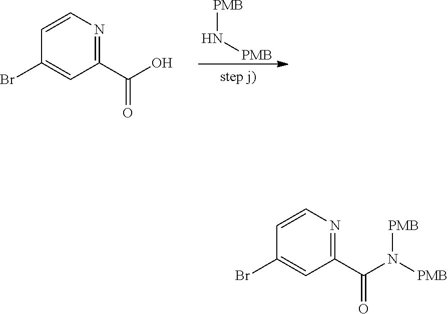

Bis-(4-methoxybenzyl)-amine (636 mg, 2.47 mmol), HATU (1130 mg, 2.97 mmol) and N, N-diisopropylethylamine (638 mg, 4.95 mmol) were added to a solution of 4-bromopyridine carboxylic acid (500 mg, 2.47 mmol) in N,N-dimethylformamide (6 mL) and reacted at room temperature for 16 h. After the reaction was completed, the reaction system was quenched with water (20 mL), extracted with ethyl acetate (20 mL×3), and the combined organic phase was washed with saturated salt water (20 mL), dried with anhydrous sodium sulfate, filtered, and the filtrate was concentrated under reduced pressure. The crude product was purified by silica gel column chromatography (ethyl acetate/petroleum ether (v/v)=1/4) to obtain transparent oil 4-bromo-N,N-bis(4-methoxybenzyl)pyridine-2-carboxamide (500 mg, yield 45%). ¹H NMR (400 MHz, DMSO-d₆) δ 8.49 (d, J=5.4 Hz, 1H), 7.90 (d, J=1.8 Hz, 1H), 7.77 (dd, J=5.3, 1.9 Hz, 1H), 7.23 (d, J=8.6 Hz, 2H), 7.14 (d, J=8.6 Hz, 2H), 6.94 (2H, t, J=5.7 Hz), 6.89 (d, J=8.7 Hz, 2H), 4.48 (s, 2H), 4.34 (s, 2H), 3.76 (s, 3H), 3.74 (s, 3H). ESI-MS (m/z): 443.1 [M+H]⁺.

### Example 16

### Preparation of 2-(4,4-difluoroazepan-1-yl)-6,7-difluoro-N-(3-sulfamoylphenyl) quinoline-3-carboxamide (KH16)

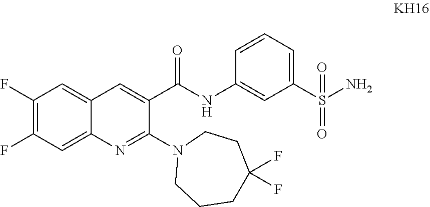 [SMILES: NS(=O)(=O)c1cccc(NC(=O)c2cc3cc(F)c(F)cc3nc2N2CCCC(F)(F)CC2)c1]

The preparation of 2-(4,4-difluoroazepan-1-yl)-6,7-difluoro-N-(3-sulfamoylphenyl) quinoline-3-carboxamide is similar to that of Example 10 step a-d and Example 6, except that 3-(4,4-difluoroazepan-1-yl)quinoxaline-2-carboxylic acid in step f of example 6 is changed into 2-(4,4-difluoroazepan-1-yl)-6,7-difluoroquinolin-3-carboxylic acid which can be synthesized by step a-d of example 10, and 4-bromo-2-fluoropyridine in step a of example 6 is replaced by 4-bromo-2-fluorobenzene, and the room temperature at step e of example 6 was changed to 80° C. to obtain a white solid 2-(4,4-difluoroazepan-1-yl)-6,7-difluoro-N-(3-sulfamoylphenyl)quinoline-3-carboxamide (10.92 mg, yield 17.5%). ¹H NMR (400 MHz, MeOD) δ 8.36 (s, 1H), 8.26 (s, 1H), 7.86 (d, J=8.1 Hz, 1H), 7.67 (dd, J=19.6, 10.0 Hz, 2H), 7.60-7.43 (m, 2H), 3.87-3.78 (m, 2H), 3.66 (2H, t, J=5.9 Hz), 2.42-2.40 (m, 2H), 2.08-2.00 (m, 2H), 1.99-1.91 (m, 2H). ESI-MS (m/z): 497.1 [M+H]⁺.

### Example 17

### Preparation of 2-(4,4-difluoroazepan-1-yl)-6,7-difluoro-N-(2-sulfamoylpyridine-4-yl)quinoline-3-carboxamide (KH17)

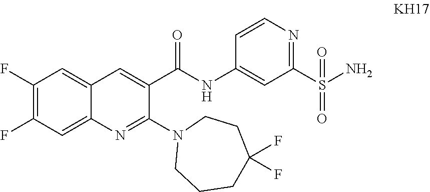 [SMILES: NS(=O)(=O)c1cc(NC(=O)c2cc3cc(F)c(F)cc3nc2N2CCCC(F)(F)CC2)ccn1]

The synthesis of 2-(4,4-difluoroazepan-1-yl)-6,7-difluoro-N-(2-sulfamoylpyridine-4-yl) quinoline-3-carboxamide is similar to step a-d of Example 10 and Example 6, except that 3-(4,4-difluoroazepan-1-yl)quinoxaline-2-carboxylic acid in step f of Example 6 is changed into 2-(4,4-difluoroazepan-1-yl)-6,7-difluoroquinolin-3-carboxylic acid which can be synthesized by step a-d of example 10, and stirring at room temperature for 2 h in step e of example 6 is changed to stirring at room temperature for 5 h. Yellow solid 2-(4,4-difluoroazepan-1-yl)-6,7-difluoro-N-(2-sulfamoylpyridine-4-yl)quinoline-3-carboxamide (13.9 mg, 24% yield) was obtained. ¹H NMR (400 MHz, DMSO-d₆) δ 11.38 (s, 1H), 8.64 (d, J=5.3 Hz, 1H), 8.41 (s, 1H), 8.32 (s, 1H), 7.92 (t, J=10.0 Hz, 1H), 7.86 (d, J=5.4 Hz, 1H), 7.60 (dd, J=11.8, 7.9 Hz, 1H), 7.47 (s, 2H), 3.73 (s, 2H), 3.54 (s, 2H), 2.40 (s, 2H), 1.99 (s, 2H), 1.89 (s, 2H). ESI-MS (m/z): 498.2 [M+H]⁺.

### Example 18

### Preparation of N-(2-carbamoylpyridin-4-yl)-7-(4,4-difluoroazepan-1-yl)quinoline-6-carboxamide (KH18)

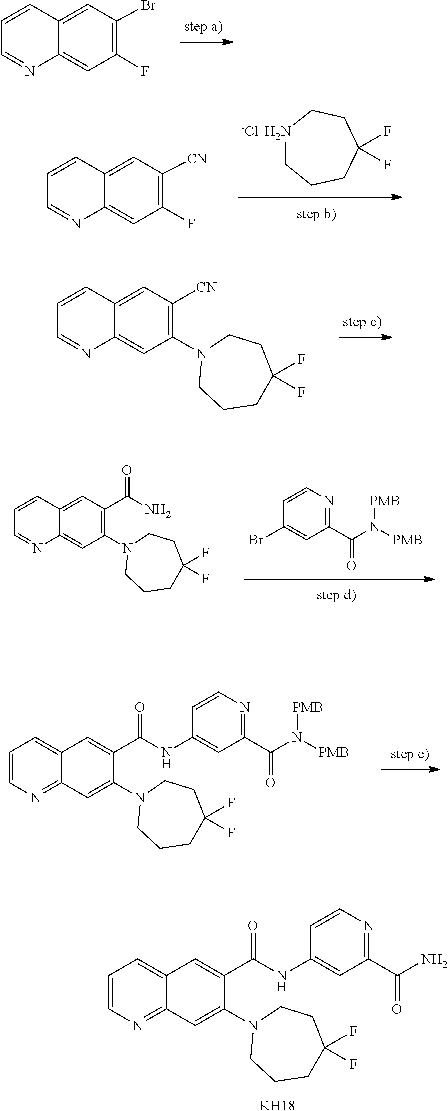

### Step a): Preparation of 7-fluoroquinoline-6-carbonitrile

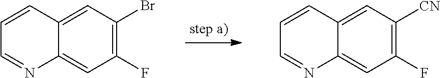 [SMILES: Fc1cc2ncccc2cc1Br ; N#Cc1cc2cccnc2cc1F]

Zn(CN)₂ (2.07 g, 17.68 mmol) and Pd(PPh₃)₄ (2.04 g, 1.77 mmol) were added to a solution of 6-bromo-7-fluoroquinoline (2 g, 8.84 mmol) in NMP (20 mL) and the reaction system was stirred at 120° C. for 16 h under nitrogen protection. After the reaction was completed, it was diluted with water, extracted with ethyl acetate (20 mL×3), and the combined organic phase was washed with saturated salt water, dried with anhydrous sodium sulfate, filtered, and the filtrate was concentrate under reduced pressure. The resulting crude product was purified by silica gel column chromatography (petroleum ether/ethyl acetate (v/v)=1/1) to obtain a light yellow solid 7-fluoroquinoline-6-carbonitrile (1.2 g, yield 78.8%). ¹H NMR (400 MHz, CDCl₃) δ 9.06 (dd, J=4.3, 1.5 Hz, 1H), 8.24 (dd, J=11.7, 3.8 Hz, 2H), 7.88 (d, J=10.0 Hz, 1H), 7.53 (dd, J=8.4, 4.3 Hz, 1H). ESI-MS (m/z): 173.0 [M+H]⁺.

### Step b): Preparation of 7-(4,4-difluoroazepan-1-yl) quinoline-6-carbonitrile

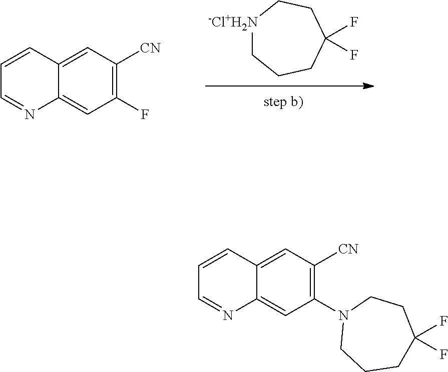

4,4-Difluoroazepane hydrochloride (4.7 g, 34.85 mmol), K₂CO₃ (4.8 g, 34.85 mmol) and DIEA (4.5 g, 34.85 mmol) were added to a solution of 7-fluoroquinolin-6-carbonitrile (1.2 g, 6.97 mmol) in DMSO (10 mL) respectively and stirred at 100° C. for 16 h. After the reaction was completed, the reaction system was diluted with water, extracted with ethyl acetate (20 mL×3), and the combined organic phase was washed with saturated salt water, dried with anhydrous sodium sulfate, filtered, and the filtrate was concentrated under reduced pressure. The resulting crude product was purified by silica gel column chromatography (petroleum ether/ethyl acetate (v/v)=3/1) to obtain light yellow solid 7-(4,4-difluoroazepan-1-l)quinoline-6-carbonitrile (800 mg, yield 24.1%). ¹H NMR (400 MHz, DMSO-d₆) delta 8.99-8.84 (m, 1H), 8.54 (s, 1H), 8.30 (d, J=8.2 Hz, 1H), 7.45 (s, 1H), 7.41 (dd, J=8.2, 4.2 Hz, 1H), 3.65-3.48 (m, 4H), 2.50-2.37 (m, 2H), 2.32-2.14 (m, 2H), 2.06-1.88 (m, 2H). ESI-MS (m/z): 288.0 [M+H]⁺.

### Step c): Preparation of 7-(4,4-difluoroazepan-1-yl)quinoline-6-carboxamide

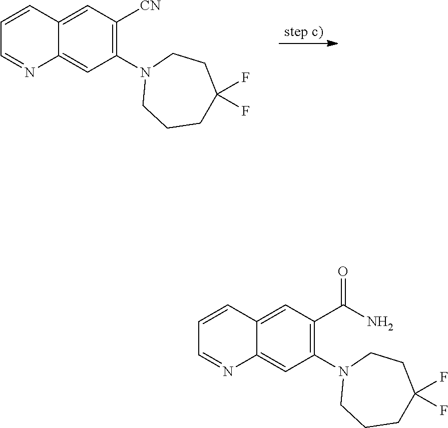 [SMILES: N#Cc1cc2cccnc2cc1N1CCCC(F)(F)CC1 ; NC(=O)c1cc2cccnc2cc1N1CCCC(F)(F)CC1]

Cesium carbonate (3.16 g, 9.72 mmol) was added to a solution of 7-(4,4-difluoroazepan-1-yl)quinoline-6-carbonitrile (800 mg, 2.77 mmol) in dimethyl sulfoxide (10 mL), and 30% hydrogen peroxide (2.5 mL) was slowly added at 0° C. Reaction was performed at room temperature for 4 h. After the reaction was completed, the reaction system was quenched with ice water (10 mL), extracted with ethyl acetate (60 mL×2), and the combined organic phase was washed with saturated salt water (60 mL), dried with anhydrous sodium sulfate, and filtered. The filtrate was concentrated under reduced pressure to obtain white solid 7-(4,4-difluoroazepan-1-yl) quinoline-6-carboxamide (80 mg, yield 95%). ESI-MS (m/z): 306.1 [M+H]⁺.

### Step d): Preparation of N-(2-(bis(4-methoxybenzyl)carbamoyl)pyridin-4-yl)-7-(4,4-difluoroazepan-1-yl)quinoline-6-carboxamide

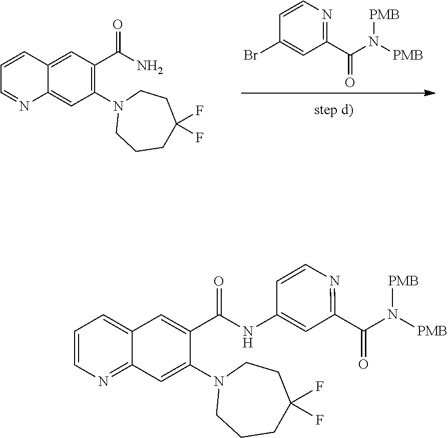

For the synthesis of 4-bromo-N,N-bis(4-methoxybenzyl)pyridine-2-carboxamide, see step j of example 15.

4-Bromo-N,N-bis(4-methoxybenzyl)pyridine-2-carboxamide (530 mg, 1.19 mmol), Brettphos-Pd-G3 (98.7 mg, 0.109 mmol) and cesium carbonate (880 mg, 2.72 mmol) were added to a solution of 7-(4,4-difluoroazepan-1-yl)quinoline-6-carboxamide (350 mg, 1.06 mmol) in toluene (5 mL) and stirred at 110° C. for 16 h under nitrogen protection. After the reaction was completed, the reaction system was quenched with water (50 mL), extracted with ethyl acetate (15 mL×3), and the combined organic phase was washed with saturated salt water (10 mL×3), dried with anhydrous sodium sulfate, filtered, and the filtrate was concentrated under reduced pressure. The crude products was purified by silica gel column chromatography purification (ethyl acetate/petroleum ether (v/v)=1/1) to obtain yellow solid N-(2-(bis(4-methoxybenzyl)carbamoyl)pyridine-4-yl)-7-(4,4-difluoroazepan-1-yl)quinoline-6-carboxamide (200 mg, yield 26.2%). ¹H NMR (400 MHz, DMSO-d₆) δ 11.13 (s, 1H), 8.86 (dd, J=4.3, 1.7 Hz, 1H), 8.54 (d, J=5.6 Hz, 1H), 8.41-8.26 (m, 1H), 8.14 (s, 1H), 8.01 (d, J=1.7 Hz, 1H), 7.77 (dd, J=5.6, 2.0 Hz, 1H), 7.50 (s, 1H), 7.39 (dd, J=8.2, 4.3 Hz, 1H), 7.20 (dd, J=21.2, 8.6 Hz, 4H), 6.92 (dd, J=18.3, 8.6 Hz, 4H), 4.47 (s, 2H), 4.41 (s, 2H), 3.75 (d, J=10.4 Hz, 6H), 3.42 (t, J=5.8 Hz, 4H), 2.34-2.18 (m, 2H), 2.08 (dd, J=16.0, 10.4 Hz, 2H), 1.83 (d, J=5.3 Hz, 2H). ESI-MS (m/z): 665.9 [M+H]⁺.

### Step e): Preparation of N-(2-carbamoylpyridine-4-yl)-7-(4,4-difluoroazepan-1-yl)quinoline-6-carboxamide

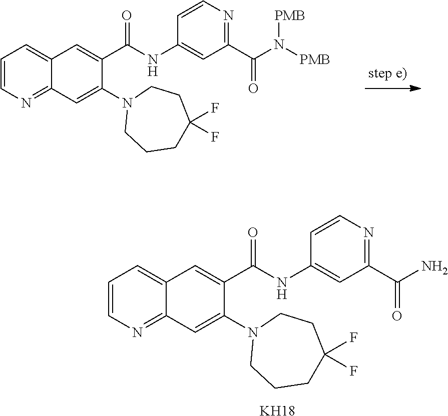

Trifluoroacetic acid (2 mL) was added to a solution of N-(2-(bis(4-methoxybenzyl)carbamoyl)pyridine-4-yl)-7-(4,4-difluoroazepan-1-yl)quinoline-6-carboxamide (200 mg, 0.30 mmol) in dichloroethane (2 mL). The reaction system was stirred at 80° C. for 2 H. After the reaction was completed, the reaction mixture was concentrated and the resulting crude product was purified by Prep-HPLC (1% formic acid aqueous solution, methanol) to obtain white solid N-(2-carbamoylpyridine-4-yl)-7-(4,4-difluoroazepan-1-yl)quinoline-6-carboxamide (40 mg, yield 31.2%). ¹H NMR (400 MHz, DMSO-d₆) δ 11.33 (s, 1H), 9.02 (d, J=4.0 Hz, 1H), 8.79 (d, J=7.8 Hz, 1H), 8.58 (d, J=5.5 Hz, 1H), 8.45-8.30 (m, 2H), 8.12 (s, 1H), 7.92 (dd, J=5.4, 2.0 Hz, 1H), 7.69-7.60 (m, 2H), 7.43 (s, 1H), 3.55 (dd, J=10.5, 4.6 Hz, 4H), 2.37-2.35 (m, 2H), 2.13-2.07 (m, 2H), 1.95-1.87 (m, 2H). ESI-MS (m/z): 426.0 [M+H]⁺.

### Example 19

### Preparation of N-(3-carbamoyl-4-fluorophenyl)-7-(4,4-difluoroazepan-1-yl)quinoline-6-carboxamide (KH19)

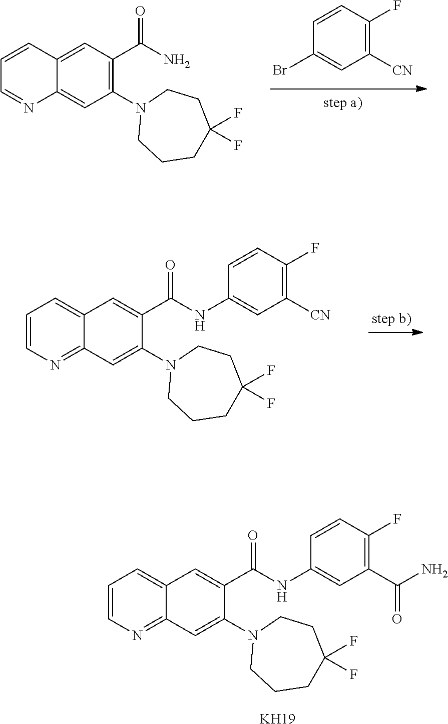

### Step a): Preparation of N-(3-cyano-4-fluorophenyl)-7-(4,4-difluoroazepan-1-yl)quinoline-6-carboxamide

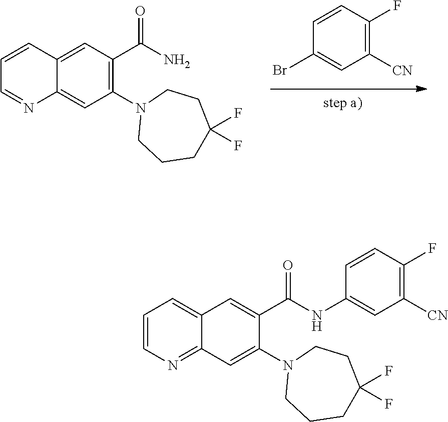 [SMILES: NC(=O)c1cc2cccnc2cc1N1CCCC(F)(F)CC1 ; N#Cc1cc(Br)ccc1F ; N#Cc1cc(NC(=O)c2cc3cccnc3cc2N2CCCC(F)(F)CC2)ccc1F]

For the synthesis of 7-(4,4-difluoroazepan-1-yl)quinoline-6-carboxamide, see step a-c of Example 18. 5-Bromo-2-fluorobenzonitrile (238 mg, 1.19 mmol), Brettphos-Pd-G3 (98.7 mg, 0.109 mmol) and cesium carbonate (880 mg, 2.72 mmol) were added to a solution of 7-(4,4-difluoroazepan-1-yl)quinoline-6-carboxamide (350 mg, 1.06 mmol) in toluene (5 mL) and stirred at 110° C. for 16 h under nitrogen protection. After the reaction was completed, the reaction system was quenched with water (50 mL), extracted with ethyl acetate (15 mL×3), and the combined organic phase was washed with saturated salt water (10 mL×3), dried with anhydrous sodium sulfate, filtered, and the filtrate was concentrated under reduced pressure. The crude product was purified by silica gel column chromatography (ethyl acetate/petroleum ether (v/v)=1/1) to obtain yellow solid N-(3-cyano-4-fluorophenyl)-7-(4,4-difluoroazepan-1-yl)quinoline-6-carboxamide (200 mg, yield 44.3%). ESI-MS (m/z): 424.9 [M+H]⁺.

### Step b): Preparation of N-(3-carbamoyl-4-fluorophenyl)-7-(4,4-difluoroazepan-1-yl)quinoline-6-carboxamide (KH19)

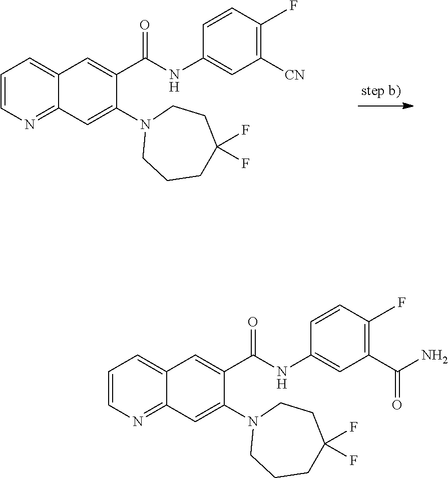 [SMILES: N#Cc1cc(NC(=O)c2cc3cccnc3cc2N2CCCC(F)(F)CC2)ccc1F ; NC(=O)c1cc(NC(=O)c2cc3cccnc3cc2N2CCCC(F)(F)CC2)ccc1F]

Cesium carbonate (535 mg, 1.64 mmol) was added to a solution of N-(3-cyano-4-fluorophenyl)-7-(4,4-difluoroazepan-1-l)quinoline-6-carboxamide (200 mg, 0.47 mmol) in dimethyl sulfoxide (5 mL), and 30% hydrogen peroxide (0.5 mL) was slowly added at 0° C., and reacted at room temperature for 4 h. After the reaction was completed, the reaction system was quenched with ice water (10 mL), extracted with ethyl acetate (20 mL×3), and the combined organic phase was washed with saturated salt water (60 mL), and then dried with anhydrous sodium sulfate, filtered, and the filtrate was concentrated under reduced pressure. The resulting crude product was purified by Prep-HPLC (1% formic acid aqueous solution, methanol) to obtain white solid N-(3-carbamoyl-4-fluorophenyl)-7-(4,4-difluoroazepan-1-yl)quinoline-6-carboxamide (22.12 mg, yield 30.56%). ¹H NMR (400 MHz, DMSO-d₆) δ 10.91 (s, 1H), 8.99 (d, J=3.9 Hz, 1H), 8.75 (d, J=8.1 Hz, 1H), 8.30 (s, 1H), 8.05 (dd, J=6.4, 2.7 Hz, 1H), 7.92-7.80 (m, 1H), 7.72-7.54 (m, 3H), 7.40 (s, 1H), 7.31 (d, J=9.2 Hz, 1H), 3.67-3.43 (m, 4H), 2.35-2.30 (m, 2H), 2.13-2.09 (m, 2H), 1.91 (br s, 2H). ESI-MS (m/z): 443.3 [M+H]⁺.

### Example 20

### Preparation of N-(2-carbamoylpyridin-4-yl)-6-chloro-3-(4,4-difluoroazepan-1-yl)quinoline-2-carboxamide (KH20)

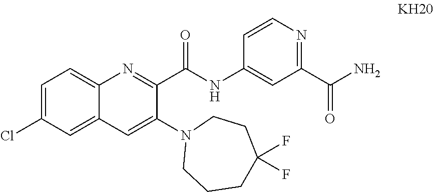 [SMILES: NC(=O)c1cc(NC(=O)c2nc3ccc(Cl)cc3cc2N2CCCC(F)(F)CC2)ccn1]

The synthesis of N-(2-carbamoylpyridin-4-yl)-6-chloro-3-(4,4-difluoroazepan-1-yl) quinoline-2-carboxamide is similar to that of example 15, except that 2-methyl-1H-indole in step a is replaced with 5-chloro-2-methyl-1H-indole. Yellow solid N-(2-carbamoylpyridin-4-yl)-6-chloro-3-(4,4-difluoroazepan-1-yl)quinoline-2-carboxamide (6.67 mg, 18% yield) was obtained. ¹H NMR (400 MHz, DMSO-d₆) δ 11.38 (s, 1H), 8.57 (d, J=5.4 Hz, 1H), 8.42 (d, J=1.8 Hz, 1H), 8.12 (s, 1H), 8.02 (d, J=2.3 Hz, 1H), 7.98 (d, J=8.9 Hz, 1H), 7.92 (dd, J=5.5, 2.1 Hz, 1H), 7.88 (s, 1H), 7.67 (s, 1H), 7.57 (dd, J=8.9, 2.3 Hz, 1H), 3.47-3.40 (m, 4H), 2.29-2.24 (m, 2H), 2.16-2.04 (m, 2H), 1.87 (br s, 2H). ESI-MS (m/z): 460.1 [M+H]⁺.

### Example 21

### Preparation of N-(3-carbamoyl-4-fluorophenyl)-2-(4,4-difluoroazepan-1-yl)-6,7-difluoroquinoline-3-carboxamide (KH21)

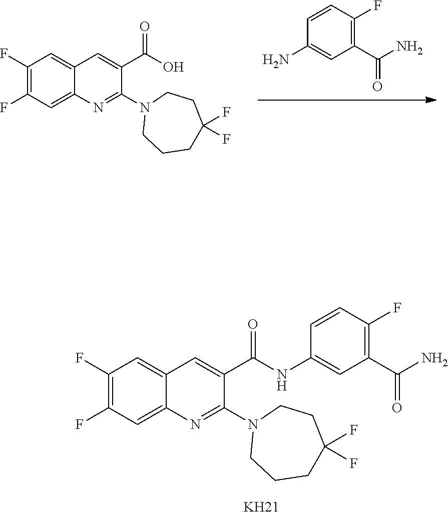

2-(4,4-Difluoroazepan-1-yl)-6,7-difluoroquinoline-3-carboxylic acid was prepared in the same way as step a-d of Example 10.

HATU (187.7 mg, 0.494 mmol), DIEA (196.1 mg, 1.520 mmol) and 5-amino-2-fluorobenzamide (58.5 mg, 0.380 mmol) were added to a solution of 2-(4,4-difluoroazepan-1-yl)-6,7-difluoroquinolin-3-carboxylic acid (130 mg, 0.380 mmol) in dichloromethane (4 mL) and DMF (1 mL) at room temperature and reacted at 40° C. for 48 h. After the reaction was completed, water was added to quench the reaction system, and the organic phase (15 mL) was extracted with ethyl acetate, washed with saturated salt water, dried with anhydrous sodium sulfate and filtered, and the filtrate was concentrated under reduced pressure. The crude product was prepared by Prep-HPLC to obtain white solid N-(3-carbamoyl-4-fluorophenyl)-2-(4,4-difluoroazepan-1-yl)-6,7-difluoroquinoline-3-carboxamide (64.17 mg, yield 35%). ¹H NMR (400 MHz, DMSO-d₆) δ 10.83 (s, 1H), 8.31 (s, 1H), 8.11-8.00 (m, 1H), 7.95-7.88 (m, 1H), 7.86-7.81 (m, 1H), 7.70 (s, 2H), 7.60-7.57 (m, 1H), 7.30 (t, J=9.5 Hz, 1H), 3.74 (br s, 2H), 3.59 (t, J=5.7 Hz, 2H), (2.49 2.39 m, 2H), 2.02-1.90 (m, 2H), 1.89 (brs, 2H). ESI-MS (m/z): 479.2 [M+H]⁺.

### Example 22

### Preparation of N-(2-carbamoylpyridin-4-yl)-2-(4,4-difluoroazepan-1-yl)-7-methoxyquinoline-3-carboxamide (KH22)

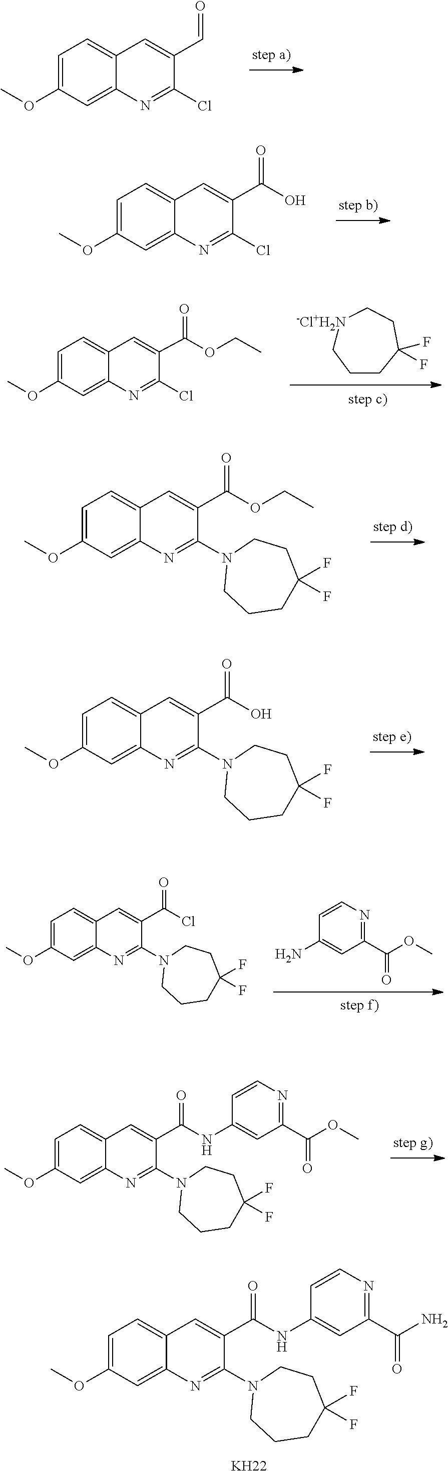

### Step a): Preparation of 2-chloro-7-methoxyquinoline-3-carboxylic acid

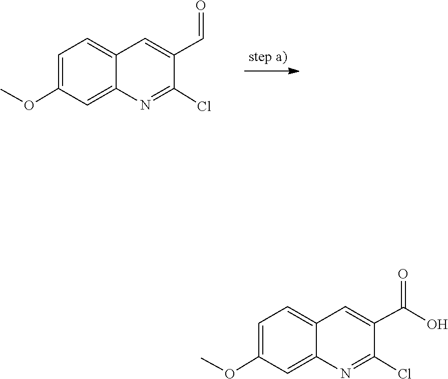 [SMILES: COc1ccc2cc(C=O)c(Cl)nc2c1 ; COc1ccc2cc(C(=O)O)c(Cl)nc2c1]

Ethanol/water (v/v=4/1, 10 mL) solution of silver nitrate (2.45 g, 14.44 mmol) and sodium hydroxide (1.80 g, 45.12 mmol) were added to the solution of 2-chloro-7-methoxyquinoline-3-carbaldehyde (2.0 g, 9.02 mmol) in ethanol (20 mL) and stirred at room temperature for 16 h. After the reaction was completed, the reaction system was diluted with 20 mL water, then extracted with ethyl acetate (30 mL×3), and the combined organic phase was washed with saturated salt water, dried with anhydrous sodium sulfate, filtered, and the filtrate was concentrated under reduced pressure and crude brown solid 2-chloro-7-methoxyquinoline-3-carboxylic acid was obtained (1.0 g, yield 50%). ¹HNMR (400 MHz, DMSO-d₆) δ 13.58 (brs, 1H), 8.86 (s, 1H), 8.08 (d, J=9.0 Hz, 1H), 7.41 (d, J=2.4 Hz, 1H), 7.38-7.32 (m, 1H), 3.95 (s, 3H). ESI-MS (m/z): 237.8 [M+H]⁺.

### Step b): Preparation of ethyl 2-chloro-7-methoxyquinoline-3-carboxylate

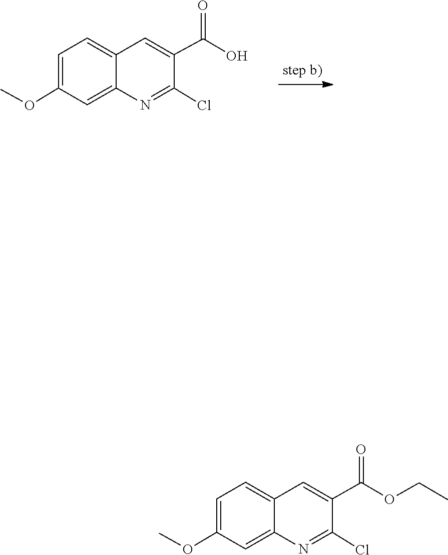 [SMILES: COc1ccc2cc(C(=O)O)c(Cl)nc2c1 ; CCOC(=O)c1cc2ccc(OC)cc2nc1Cl]

2-Chloro-7-methoxyquinoline-3-carboxylic acid (1.0 g, 4.21 mmol) was dissolved in DMF (15 mL), and K₂CO₃ (1.16 g, 8.42 mmol) was added. The reaction system was placed in a 0° C. ice water bath and then ethane iodide was added (1.31 g, 8.42 mmol). After that, the ice water bath was removed and the reaction was carried out at room temperature for 2 h. After the reaction was completed, the reaction system was diluted with 20 mL water, extracted by ethyl acetate (30 mL×3), and the combined organic phase was washed in saturated salt water, dried with anhydrous sodium sulfate, and filtered, and the filtrate was concentrated under reduced pressure. The crude product was purified by silica gel column chromatography (petroleum ether/ethyl acetate (v/v)=8/1) to obtain white solid ethyl 2-chloro-7-methoxyquinoline-3-carboxylate (600 mg, yield 60%). ESI-MS (m/z) 265.9 [M+H]⁺.

### Step c): Preparation of ethyl 2-(4,4-difluoroazepan-1-yl)-7-methoxyquinoline-3-carboxylate

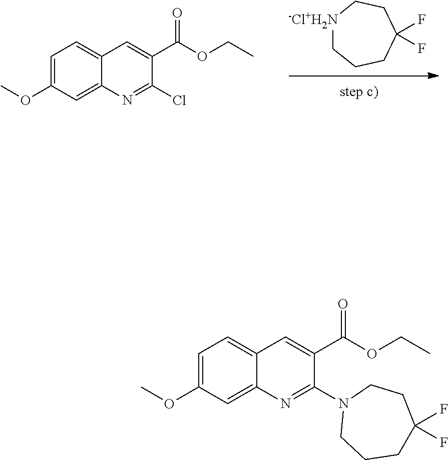

4,4-Difluoroazepane hydrochloride (503.82 mg, 2.94 mmol) and N,N-diisopropylethylamine (875.61 g, 6.77 mmol) were added to a solution of ethyl 2-chloro-7-methoxyquinoline-3-carboxylate (600 mg, 2.26 mmol) in N-methylpyrrolidone (10 mL). After the reaction was completed, the reaction system was diluted with water (30 mL), then extracted with ethyl acetate (20 mL×2), and the combined organic phase was washed with saturated salt water (20 mL), dried with anhydrous sodium sulfate and filtered, and the filtrate was concentrated under reduced pressure. The crude product was purified by silica gel column chromatography (ethyl acetate/petroleum ether (v/v)=1/8) to obtain colorless oil ethyl 2-(4,4-difluoroazepan-1-yl)-7-methoxyquinoline-3-carboxylate (400 mg, yield 66.7%). ESI-MS (m/z): 365.5 [M+H]⁺.

### Step d): Preparation of 2-(4,4-difluoroazepan-1-yl)-7-methoxyquinoline-3-carboxylic acid

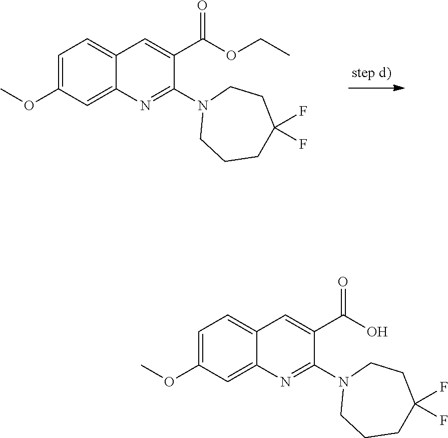 [SMILES: CCOC(=O)c1cc2ccc(OC)cc2nc1N1CCCC(F)(F)CC1 ; COc1ccc2cc(C(=O)O)c(N3CCCC(F)(F)CC3)nc2c1]

Lithium hydroxide (262.87 mg, 10.98 mmol) was slowly added to the solution of ethyl 2-(4,4-difluoroazepan-1-yl)-7-methoxyquinoline-3-carboxylate (400 mg, 1.1 mmol) in the mixed solvent of water (1 mL)/methanol (5 mL)/tetrahydrofuran (1 mL) and reacted at 50° C. for 12 h. After the reaction was completed, water (20 mL) was added to dilute the reaction mixture, and the pH of that was adjusted to about 4 with 1 M of dilute hydrochloric acid, then the organic phase was extracted with ethyl acetate (30 mL×3), washed with saturated salt water, dried with anhydrous sodium sulfate and filtered. The filtrate was concentrated under reduced pressure to obtain white solid 2-(4,4-difluoroazepan-1-yl)-7-methoxyquinoline-3-carboxylic acid (320 mg, yield 80%). ESI-MS (m/z) 336.9 [M+H]⁺.

### Step e): Preparation of 2-(4,4-difluoroazepan-1-yl)-7-methoxyquinoline-3-formyl chloride

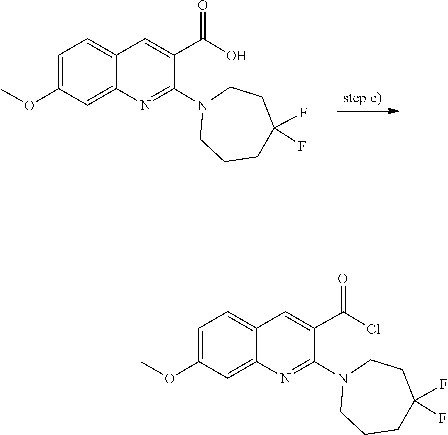 [SMILES: COc1ccc2cc(C(=O)O)c(N3CCCC(F)(F)CC3)nc2c1 ; COc1ccc2cc(C(=O)Cl)c(N3CCCC(F)(F)CC3)nc2c1]

Thionyl chloride (222.36 mg, 1.9 mmol) was added to the solution of 2-(4,4-difluoroazepan-1-yl)-7-methoxyquinoline-3-carboxylic acid (320 mg, 0.951 mmol) in dichloromethane (5 mL) at 0° C. After the reaction was completed, the reaction mixture was concentrated under reduced pressure and colorless oil 2-(4,4-difluoroazepan-1-yl)-7-methoxyquinoline-3-formyl chloride (320 mg, yield 100%) was obtained, which did not need purification and was directly used in the next step. ESI-MS (m/z): 335.6 [M+H]⁺.

### Step f): Preparation of methyl 4-(2-(4,4-difluoroazepan-1-yl)-7-methoxyquinoline-3-carboxamido)pyridine-2-carboxylate

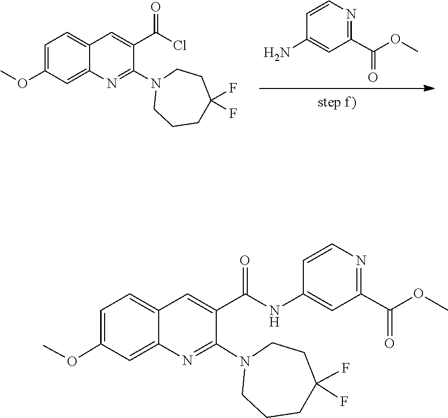

Triethylamine (182.54 mg, 1.8 mmol) and methyl 4-amino-pyridine-2-carboxylate (205.85 mg, 1.35 mmol) were added to a solution of 2-(4,4-difluoroazepan-1-yl)-7-methoxyquinoline-3-formyl chloride (320 mg, 0.9 mmol) in dichloromethane (5 mL) at 0° C. After the reaction was completed, the reaction mixture was diluted with water (20 mL), then extracted with ethyl acetate (20 mL×2), and the combined organic phase was washed with saturated salt water (20 mL), dried with anhydrous sodium sulfate and filtered, and the filtrate was concentrated under reduced pressure. The crude product was purified by silica gel column chromatography (ethyl acetate/petroleum ether (v/v)=1/1) to obtain yellow solid methyl 4-(2-(4,4-difluoroazepan-1-yl)-7-methoxyquinoline-3-carboxamido)pyridine-2-carboxylate (140 mg, yield 43.75%). ¹HNMR (400 MHz, CDCl₃) δ 8.71-8.67 (m, 2H), 8.24 (s, 1H), 8.05 (s, 1H), 7.72 (d, J=8.9 Hz, 1H), 7.23-7.21 (m, 1H), 7.12 (d, J=9.0 Hz, 1H), 4.12 (d, J=7.2 Hz, 2H), 4.03 (s, 3H), 3.99-3.97 (m, 3H), 3.77-3.73 (m, 2H), 3.57-3.53 (m, 2H), 2.22 (br s, 2H), 1.97 (br s, 2H). ESI-MS (m/z): 470.8 [M+H]⁺.

### Step g): Preparation of N-(2-carbamoylpyridin-4-yl)-2-(4,4-difluoroazepan-1-yl)-7-methoxyquinoline-3-carboxamide

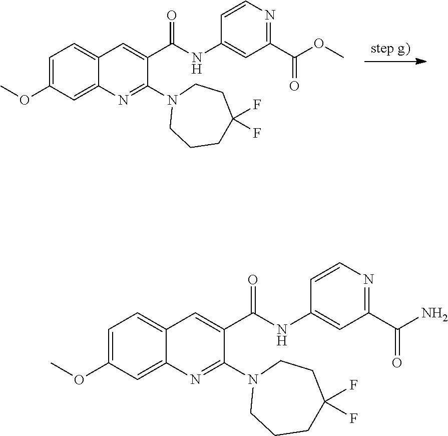

Ammonium chloride (127.33 mg, 2.38 mmol) and ammonia water (417.15 mg, 2.98 mmol) were added to a solution of methyl 4-(2-(4,4-difluoroazepan-1-yl)-7-methoxyquinoline-3-carboxamido)pyridine-2-carboxylate (140 mg, 0.297 mmol) in dimethyl sulfoxide (4 mL), and it was stirred for 24 h in a sealed tank at 70° C. After the reaction was completed, water (15 mL) was added to dilute the reaction mixture, then it was extracted with ethyl acetate (20 mL×2), and the combined organic phase was washed with saturated salt water (20 mL), dried with anhydrous sodium sulfate and filtered, and the filtrate was concentrated under reduced pressure. The resulting crude product was prepared by C18 reversed columnar formic acid to obtain yellow solid powder N-(2-carbamoylpyridin-4-yl)-2-(4,4-difluoroazepan-1-yl)-7-methoxyquinoline-3-carboxamide (52.93 mg, yield 37.8%).) ¹HNMR (400 MHz, DMSO-d₆) δ 11.14 (s, 1H), 8.54 (d, J=5.5 Hz, 1H), 8.38 (d, J=1.5 Hz, 1H), 8.31 (s, 1H), 8.10 (s, 1H), 7.91 (dd, J=5.3, 1.8 Hz, 1H), 7.75 (d, J=8.8 Hz, 1H), 7.65 (s, 1H), 7.03 (d, J=2.2 Hz, 1H), 6.95 (dd, J=8.8, 2.4 Hz, 1H), 3.89 (s, 3H), 3.77 (br s, 2H), 3.54 (t, J=5.8 Hz, 2H), 2.44-2.38 (m, 2H), 1.99-1.89 (m, 2H), 1.88-1.82 (m, 2H). ESI-MS (m/z): 456.2 [M+H]⁺.

### Example 23

### Preparation of N-(2-carbamoylpyridin-4-yl)-2-(4,4-difluoroazepan-1-yl)-7-methylquinoline-3-carboxamide (KH23)

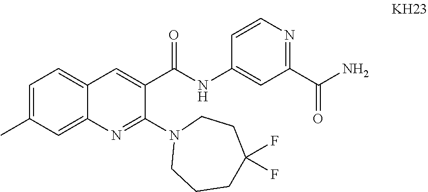 [SMILES: Cc1ccc2cc(C(=O)Nc3ccnc(C(N)=O)c3)c(N3CCCC(F)(F)CC3)nc2c1]

N-(2-carbamoylpyridin-4-yl)-2-(4,4-difluoroazepan-1-yl)-7-methylquinoline-3-carboxamide was synthesized with the same method as in Example 22, except that 2-chloro-7-methoxyquinoline-3-formaldehyde in step a is replaced by 2-chloro-7-methylquinolin-3-formaldehyde. The yellow solid N-(2-carbamoylpyridin-4-yl)-2-(4,4-difluoroazepan-1-yl)-7-methylquinoline-3-carboxamide (30.27 mg, yield 21%) was obtained by replacing the smothered tank 70° C. in step g with smothered tank 80° C. ¹H NMR (400 MHz, DMSO-d₆) δ 11.16 (s, 1H), 8.55 (d, J=5.5 Hz, 1H), 8.38 (d, J=2.0 Hz, 1H), 8.33 (s, 1H), 8.10 (s, 1H), 7.90 (d, J=5.4, 2.1 Hz, 1H), 7.74 (d, J=8.2 Hz, 1H), 7.64 (s, 1H), 7.46 (s, 1H), 7.16 (d, J=8.3, 1.5 Hz, 1H), 3.76-3.70 (m, 2H), 3.55 (2H, t, J=5.9 Hz), 2.46 (br s, 3H), 2.45-2.31 (m, 2H), 2.04-1.99 (m, 2H), (1.90 1.81 m, 2H). ESI-MS (m/z): 440.3 [M+H]⁺.

### Example 24

### Preparation of N-(2-carbamoylpyridin-4-yl)-2-(4,4-difluoroazepan-1-yl)-6,7-dimethoxyquinoline-3-carboxamide (KH24)

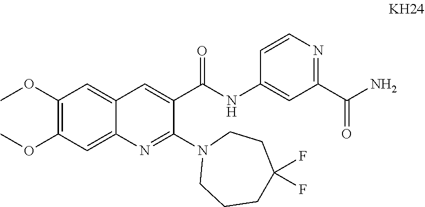 [SMILES: COc1cc2cc(C(=O)Nc3ccnc(C(N)=O)c3)c(N3CCCC(F)(F)CC3)nc2cc1OC]

N-(2-carbamoylpyridin-4-yl)-2-(4,4-difluoroazepan-1-yl)-6,7-dimethoxyquinoline-3-carboxamide was synthesized with the same method as that in Example 22, except that 2-chloro-7-methoxyquinoline-3-formaldehyde in step a) is replaced by 2-chloro-6,7-dimethoxyquinoline-3-formaldehyde. The yellow solid N-(2-carbamoylpyridin-4-yl)-2-(4,4-difluoroazepan-1-yl)-6,7-dimethoxyquinoline-3-carboxamide (110.22 mg, yield 56%) was obtained by replacing tank 70° C. in step g with tank 85° C. ¹H NMR (400 MHz, DMSO-d₆) δ 11.09 (s, 1H), 8.54 (d, J=5.5 Hz, 1H), 8.39 (s, 1H), 8.22 (s, 1H), 8.10 (s, 1H), 7.90 (d, J=3.3 Hz, 1H), 7.64 (s, 1H), 7.28 (s, 1H), 7.06 (s, 1H), 3.91 (s, 3H), 3.84 (s, 3H), 3.68 (br s, 2H), 3.51 (t, J=5.8 Hz, 2H), 2.40 (br s, 2H), 1.99-1.95 (m, 2H), 1.90-1.83 (m, 2H). ESI-MS (m/z): 486.2 [M+H]⁺.

### Example 25

### Preparation of N-(2-carbamoylpyridin-4-yl)-2-(4,4-difluoroazepan-1-yl)-7-methoxyquinoline-3-carboxamide (KH25)

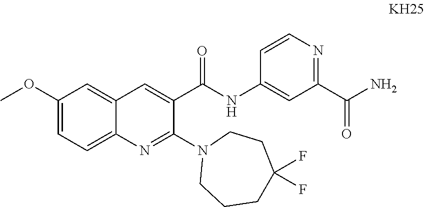 [SMILES: COc1ccc2nc(N3CCCC(F)(F)CC3)c(C(=O)Nc3ccnc(C(N)=O)c3)cc2c1]

N-(2-carbamoylpyridin-4-yl)-2-(4,4-difluoroazepan-1-yl)-7-methoxyquinoline-3-carboxamide was synthesized with the same method as that in Example 22, except that 2-chloro-7-methoxyquinoline-3-formaldehyde in step a is replaced by 2-chloro-6-methoxyquinoline-3-formaldehyde. The yellow solid N-(2-carbamoylpyridin-4-yl)-2-(4,4-difluoroazepan-1-yl)-7-methoxyquinoline-3-carboxamide (35.20 mg, yield 28%) was obtained by replacing tank 70° C. in step g with tank 85° C. ¹H NMR (400 MHz, DMSO-d₆) δ 11.16 (s, 1H), 8.55 (d, J=5.4 Hz, 1H), 8.39 (s, 1H), 8.30 (s, 1H), 8.12 (d, J=13.3 Hz, 1H), 7.89 (d, J=1.9 Hz, 1H), 7.64 (d, J=13.8 Hz, 1H), 7.60 (d, J=9.3 Hz, 1H), 7.34 (d, J=2.9 Hz, 1H), 7.31 (s, 1H), 3.84 (s, 3H), 3.68 (m, 2H), 3.53 (2H, t, J=5.9 Hz), 2.35 (m, 2H), 1.99 (m, 2H), 1.86 (m, 2H). ESI-MS (m/z): 456.1 [M+H]⁺.

Among them, the synthesis method of 2-chloro-6-methoxyquinoline-3-formaldehyde is as follows.

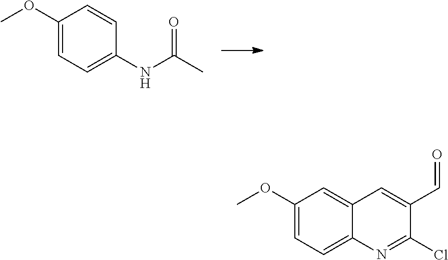

Phosphorus oxytrichloride (41.77 g, 272.41 mmol) was slowly added to DMF (7.96 g, 108.96 mmol) at 0° C., and it was stirred continually for 1 hour. p-Methoxyacetanilide (4.5 g, 27.24 mmol) was added into the reaction mixture, and the temperature was raised to 80° C. and the reaction system was continually stirred for 12 hours. After the reaction was completed, the reaction system was quenched with ice water, and then filtered after stirring for 10 minutes, the filter cake was washed with water, and the brown solid 2-chloro-6-methoxyquinoline-3-formaldehyde (1.8 g, yield 40%) crude product was obtained after being dried, which was directly used in the next step without purification. ¹H NMR (400 MHz, DMSO-d₆) δ 8.76 (s, 1H), 8.13 (d, J=8.1 Hz, 1H), 7.98 (d, J=8.4 Hz, 1H), 7.88 (t, J=7.6 Hz, 1H), 7.70 (t, J=7.5 Hz, 1H), 3.69 (s, 3H). ESI-MS (m/z): 221.9 [M+H]⁺.

### Example 26

### Preparation of 2-(4,4-difluoroazepan-1-yl)-N-(2-(S-methylsulfonimidoyl)pyridin-4-yl)quinoline-3-carboxamide (KH26)

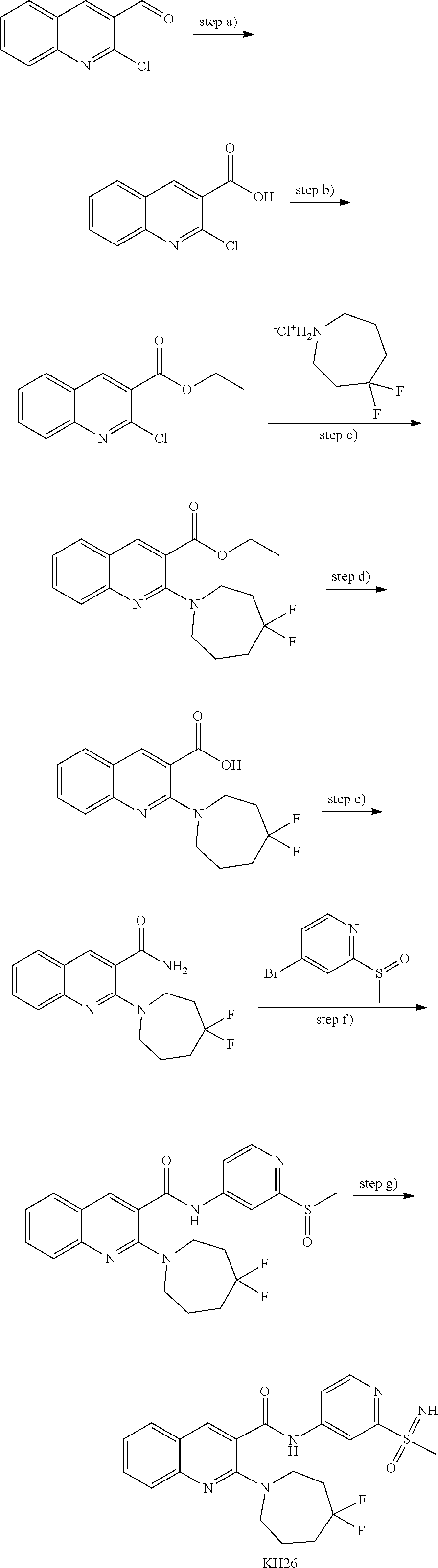

### Step a): Preparation of 2-chloroquinoline-3-carboxylic acid

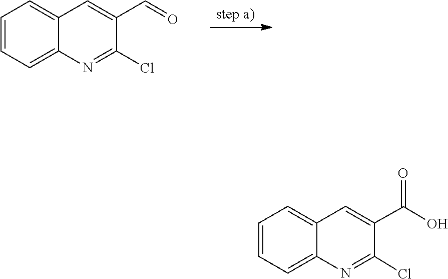 [SMILES: O=Cc1cc2ccccc2nc1Cl ; O=C(O)c1cc2ccccc2nc1Cl]

Sodium hydroxide (14.61 g, 365.32 mmol) aqueous solution was added to silver nitrate (29.61 g, 174.31 mmol) aqueous solution, followed by 2-chloroquinoline-3-formaldehyde (20 g, 104.38 mmol) and stirred at 0° C. for 3 h. After the reaction was completed, the reaction liquid was filtered, and the filtrate was adjusted to pH=5 with 3 N HCl, filtered, and yellow filter cake was obtained, that is, 2-chloroquinoline-3-carboxylic acid crude product (15 g, yield 69.2%), which was directly used in the next step. ¹H NMR (400 MHz, DMSO-d₆) δ 10.38 (s, 1H), 8.85 (s, 1H), 7.96 (d, J=9.2 Hz, 1H), 7.71 (d, J=2.8 Hz, 1H), 7.63 (dd, J=9.2, 2.8 Hz, 1H). ESI-MS (m/z): 207.8 [M+H]⁺.

### Step b): Preparation of ethyl 2-chloroquinoline-3-carboxylate

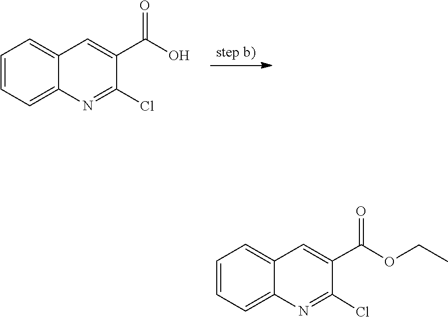 [SMILES: O=C(O)c1cc2ccccc2nc1Cl ; CCOC(=O)c1cc2ccccc2nc1Cl]

K₂CO₃ (30.62 g, 221.57 mmol) was added to a solution of 2-chloroquinoline-3-carboxylic acid (23 g, 110.78 mmol) in DMF (200 mL), and ethane iodide (34.55 g, 221.57 mmol) was dropwise added at 0° C., and then the reaction system rose to room temperature and the reaction was continued for 16 h. After the reaction was completed, 200 mL water was added to dilute the reaction mixture, and then it was extracted with ethyl acetate (200 mL×3), and the combined organic phase was washed with saturated salt water, dried with anhydrous sodium sulfate and filtered. The filtrate was concentrated under reduced pressure, and the crude product was purified by silica gel column chromatography (petroleum ether/ethyl acetate (v/v)=9/1) to obtain yellow solid ethyl 2-chloroquinoline-3-carboxylate (14 g, yield 53.6%). ¹H NMR (400 MHz, DMSO-d₆) δ 8.96 (s, 1H), 8.22-8.18 (m, 1H), 8.05-8.01 (m, 1H), 7.96 (m, 1H), 7.76 (m, 1H), (4.48 4.37 m, 2H), 1.41-1.34 (m, 3H). ESI-MS (m/z): 235.9 [M+H]⁺.

### Step c): Preparation of ethyl 2-(4,4-difluoroazepan-1-yl)quinoline-3-carboxylate

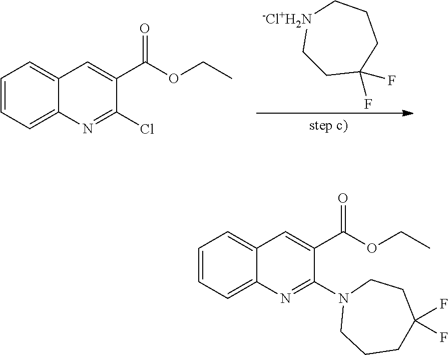

4,4-Difluoroazepane hydrochloride (28.05 g, 165.00 mmol) and DIEA (49.15 g, 381.00 mmol) were added to a solution of ethyl 2-chloroquinolin-3-carboxylate (30 g, 127.00 mmol) in NMP (400 ml) and reacted at 140° C. for 16 h. After the reaction was completed, 500 mL water was added to dilute the reaction system, which was extracted with ethyl acetate (600 mL×3), and the combined organic phase was washed three times with saturated salt water, dried with anhydrous sodium sulfate and filtered. The filtrate was concentrated under reduced pressure, and the resulting crude product was purified by silica gel column chromatography (petroleum ether/ethyl acetate (v/v)=3/1) to obtain yellow solid ethyl 2-(4,4-difluoroazepan-1-yl)quinoline-3-carboxylate (35 g, yield 82.23%). ¹H NMR (400 MHz, DMSO-d₆) δ1 8.42 (d, J=7.0 Hz, 1H), 7.84 (d, J=8.0 Hz, 1H), 7.69-7.54 (m, 2H), 7.35-7.19 (m, 1H), 4.35 (q, J=7.1 Hz, 2H), 3.78-3.63 (m, 2H), 3.44 (2H, t, J=5.8 Hz), 2.48-2.34 (m, 2H), 2.08-1.85 (m, 4H), 1.34 (t, J=7.1 Hz, 3H). ESI-MS (m/z): 335.1 [M+H]⁺.

### Step d): Preparation of 2-(4,4-difluoroazepan-1-yl)quinoline-3-carboxylic acid

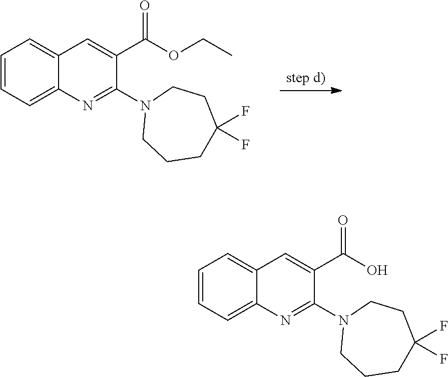 [SMILES: CCOC(=O)c1cc2ccccc2nc1N1CCCC(F)(F)CC1 ; O=C(O)c1cc2ccccc2nc1N1CCCC(F)(F)CC1]

Lithium hydroxide (11.86 g, 495.00 mmol) was added to methanol (400 mL)/water (80 mL) solution of ethyl 2-(4,4-difluoroazepan-1-yl)quinoline-3-carboxylate (35 g, 105.00 mmol) and it was stirred at room temperature overnight. After the reaction was completed, the methanol of the reaction mixture was removed by vacuum rotary evaporation, and the pH of the resulting mixture was adjusted to 3˜4 with diluted hydrochloric acid, then the product was precipitated, filtered, and the filter cake was pulped with petroleum ether to obtain white solid 2-(4,4-difluoroazepan-1-yl)quinoline-3-carboxylic acid (25 g, yield 78%). ¹H NMR (400 MHz, DMSO-d₆) δ 8.43 (s, 1H), 7.84 (d, J=7.8 Hz, 1H), 7.65-7.51 (m, 2H), 7.27 (m, 1H), 3.74-3.68 (m, 2H), 3.50 (t, J=5.9 Hz, 2H), 1.99-1.90 (m, 6H). ESI-MS (m/z): 307.1 [M+H]⁺.

### Step e): Preparation of 2-(4,4-difluoroazepan-1-yl)quinoline-3-carboxamide

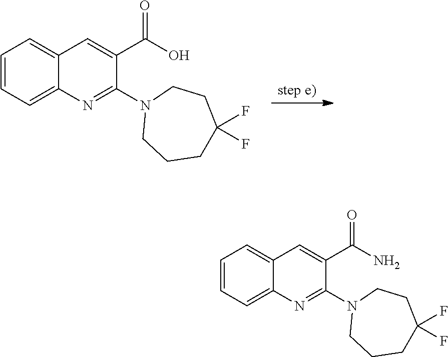 [SMILES: O=C(O)c1cc2ccccc2nc1N1CCCC(F)(F)CC1 ; NC(=O)c1cc2ccccc2nc1N1CCCC(F)(F)CC1]

Ammonium chloride (12.05 g, 225.26 mmol), DIEA (29.11 g, 225.26 mmol) and HATU (34.26 g, 90.10 mmol) were added to a solution of 2-(4,4-difluoroazepan-1-yl)quinoline-3-carboxylic acid (23 g, 75.09 mmol) in DMF (200 ml) in sequence and reacted at room temperature for 16 h. After the reaction was completed, the reaction system was diluted with 200 mL water, extracted with ethyl acetate (200 mL×3), and the combined organic phase was washed with saturated salt water, dried with anhydrous sodium sulfate and filtered. The filtrate was concentrated under reduced pressure, and the resulting crude product was purified by silica gel column chromatography (petroleum ether/ethyl acetate (v/v)=1/1) to obtain yellow solid 2-(4,4-difluoroazepan-1-yl)quinoline-3-carboxamide (9 g, yield 39.26%). ¹H NMR (400 MHz, DMSO-d₆) δ 8.15 (d, J=12.5 Hz, 2H), 7.84 (d, J=8.0 Hz, 1H), 7.64 (d, J=3.9 Hz, 2H), 7.39-7.23 (m, 1H), 3.82-3.75 (m, 2H), 3.68 (2H, t, J=6.0 Hz), 2.49-2.45 (m, 2H), 2.08 (td, J=14.3, 8.2 Hz, 2H), 2.02-1.91 (m, 2H). ESI-MS (m/z): 305.9 [M+H]⁺.

### Step f): Preparation of 2-(4,4-difluoroazepan-1-yl)-N-(2-(methylsulfinyl)pyridin-4-yl)quinoline-3-carboxamide

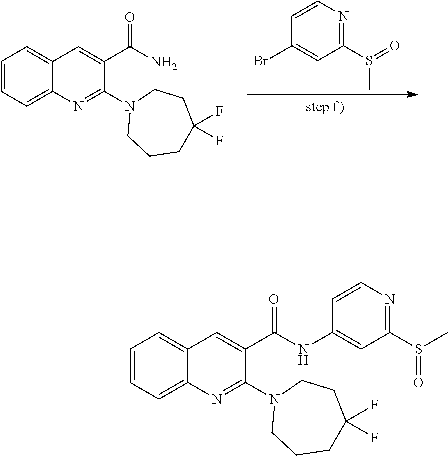

2-(4,4-Difluorazepane-1-yl)quinoline-3-carboxamide (305 mg, 1.00 mmol), 4-bromo-2-(methylsulfinyl)pyridine (220 mg, 1.00 mmol), Cs₂CO₃ (1.14 g, 3.50 mmol), Ruphos-Pd-G2 (77 mg, 0.10 mmol) were dissolved in toluene (5 mL), and the reaction system was replaced with nitrogen and stirred at 110° C. for 16 h. After the reaction was completed, 10 mL water was added to the reaction mixture, and then it was extracted with ethyl acetate (10 mL×2), and the combined organic phase was washed with saturated salt water (10 mL), dried with anhydrous sodium sulfate and filtered, and the filtrate was concentrated under reduced pressure. The crude product was purified by silica gel plate (ethyl acetate/petroleum ether (v/v)=2/1) to obtain yellow solid 2-(4,4-difluoroazepan-1-yl)-N-(2-(methylsulfinyl)pyridin-4-yl)quinoline-3-carboxamide (386 mg, yield 86.7%). ¹H NMR (400 MHz, DMSO-d₆) δ 11.41 (s, 1H), 8.65 (d, J=5.4 Hz, 1H), 8.47 (s, 1H), 8.36 (s, 1H), 7.94-7.85 (m, 2H), 7.71 (d, J=3.7 Hz, 2H), 7.41-7.35 (m, 1H), 3.86-3.75 (m, 2H), 3.63 (2H, t, J=5.8 Hz), 2.87 (s, 3H), (2.52 2.40 m, 2H), 2.13-2.05 (m, 2H), 1.98-1.90 (m, 2H). ESI-MS (m/z): 445.1 [M+H].⁺

### Step g): Preparation of 2-(4,4-difluoroazepan-1-yl)-N-(2-(S-methylsulfonimidoyl)pyridin-4-yl)quinoline-3-carboxamide

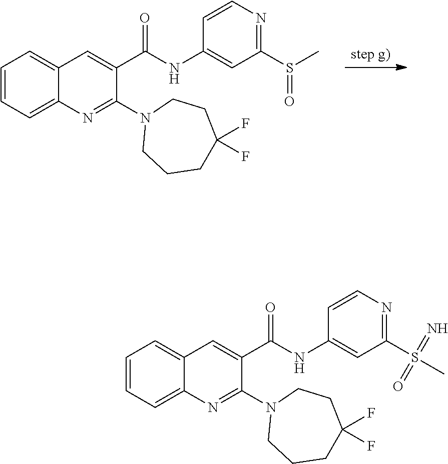 [SMILES: CS(=O)c1cc(NC(=O)c2cc3ccccc3nc2N2CCCC(F)(F)CC2)ccn1 ; CS(=N)(=O)c1cc(NC(=O)c2cc3ccccc3nc2N2CCCC(F)(F)CC2)ccn1]

2-(4,4-Difluoroazepan-1-yl)-N-(2-(methylsulfinyl)pyridin-4-yl)quinoline-3-carboxamide (350 mg, 0.786 mmol) was dissolved in Eaton's reagent (5 mL), and NaN₃ (102 mg, 1.573 mmol) was added and stirred at 50° C. for 1 H. After the reaction was completed, the reaction system was quenched with water (10 mL), extracted with dichloromethane (10 mL×2), and the combined organic phase was washed with saturated salt water (10 mL×2), dried with anhydrous sodium sulfate and filtered, and the filtrate was concentrated under reduced pressure. The resulting crude product was purified by reverse phase preparation to obtain yellow solid 2-(4,4-difluoroazepan-1-yl)-N-(2-(S-methylsulfonimidoyl)pyridin-4-yl)quinoline-3-carboxamide (47.31 mg, yield 13.1%). ¹H NMR (400 MHz, DMSO-d₆) δ 11.37 (s, 1H), 8.63 (d, J=5.4 Hz, 1H), 8.44 (d, J=1.5 Hz, 1H), 8.40 (s, 1H), 7.90 (dd, J=5.4, 1.8 Hz, 1H), 7.85 (d, J=8.0 Hz, 1H), 7.65 (d, J=3.7 Hz, 2H), 7.35-7.28 (m, 1H), 4.36 (s, 1H), 3.81-3.70 (m, 2H), 3.56 (2H, t, J=5.7 Hz), 3.16 (s, 3H), 2.47-2.35 (m, 2H), 2.08-1.95 (m, 2H), 1.93-1.83 (m, 2H). ESI-MS (m/z): 460.2 [M+H]⁺.

### Example 27

### Preparation of 2-(4,4-difluoroazepan-1-yl)-6,7-difluoro-N-(2-(S-methylsulfonimidoyl)pyridin-4-yl)quinoline-3-carboxamide (KH27)

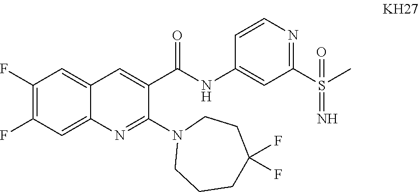 [SMILES: CS(=N)(=O)c1cc(NC(=O)c2cc3cc(F)c(F)cc3nc2N2CCCC(F)(F)CC2)ccn1]

The synthesis of 2-(4,4-difluoroazepan-1-yl)-6,7-difluoro-N-(2-(S-methylsulfonimidoyl)pyridin-4-yl) quinoline-3-carboxamide is similar to step e-g of Example 26, except that the 2-(4,4-difluoroazepan-1-yl)quinoline-3-carboxylic acid in step e is replaced with 2-(4,4-difluoroazepan-1-yl)-6,7-difluoroquinoline-3-carboxylic acid. The synthesis of 2-(4,4-difluoroazepan-1-yl)-6,7-difluoroquinoline-3-carboxylic acid is described in steps a-d of Example 10.

¹H NMR (400 MHz, DMSO-d₆) δ 11.46 (s, 1H), 8.67 (d, J=5.5 Hz, 1H), 8.46 (s, 1H), 8.40 (s, 1H), 7.95-7.90 (m, 2H), 7.62-7.57 (m, 1H), 3.73 (br s, 2H), 3.54 (2H, t, J=5.6 Hz), 3.28 (s, 3H), 2.45-2.39 (m, 2H), 2.03-1.97 (m, 2H), 1.93-1.88 (m, 2H). ESI-MS (m/z): 496.3 [M+H]⁺.

### Example 28

### Preparation of 2-(4,4-difluoroazepan-1-yl)-N-(2-(N-methylsulfamoyl)pyridin-4-yl)quinoline-3-carboxamide (KH28)

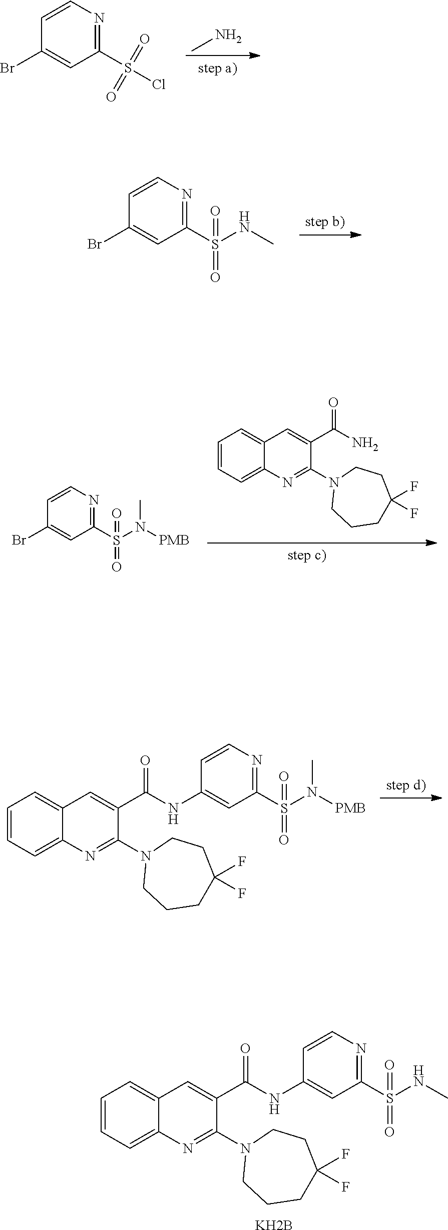

### Step a) Preparation of 4-bromo-N-methylpyridine-2-sulfonamide

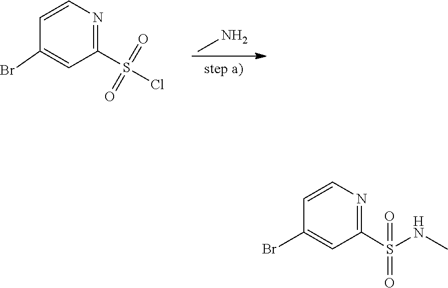

DIEA (5.786 g, 44.85 mmol) and 4-bromopyridine-2-sulfonyl chloride (2.87 g, 11.21 mmol) were added to a solution of methylamine (2.78 g, 25% aqueous solution) in tetrahydrofuran (10 mL) and it was stirred at room temperature for 2 h. After the reaction was completed, 30 mL of water was added, then it was extracted with ethyl acetate (30 mL×2), and the combined organic phase was washed with saturated salt water (30 mL), dried with anhydrous sodium sulfate and filtered, and the filtrate was concentrated under reduced pressure. The resulting crude product was purified by silica gel column chromatography (ethyl acetate/petroleum ether (v/v)=1/1). Yellow solid 4-bromo-N-methylpyridine-2-sulfonamide (1.36 g, yield 48.3%) was obtained. ¹H NMR (400 MHz, DMSO-d₆) δ 8.64 (d, J=5.2 Hz, 1H), 8.10 (d, J=1.8 Hz, 1H), 7.99 (dd, J=5.2, 1.8 Hz, 1H), 7.87-7.81 (m, 1H), 2.57 (d, J=4.9 Hz, 3H). ESI-MS (m/z): 250.9 [M+H]⁺.

### Step b) Preparation of 4-bromo-N-(4-methoxybenzyl)-N-methylpyridine-2-sulfonamide

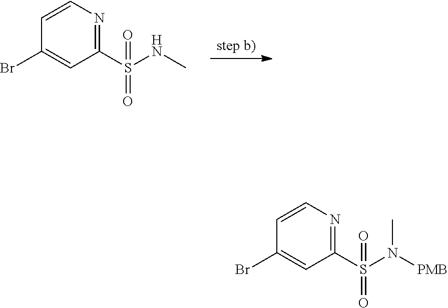

4-bromo-N-methylpyridine-2-sulfonamide (400 mg, 1.59 mmol), PMBCl (500 mg, 3.19 mmol) and K₂CO₃ (440 mg, 3.19 mmol) were dissolved in DMF (4 mL) and stirred at room temperature for 6 h. After the reaction was completed, 10 mL of water was added, then it was extracted with ethyl acetate (10 mL×3), and the combined organic phase was washed with saturated salt water (10 mL×3), dried with anhydrous sodium sulfate and filtered, and the filtrate was concentrated under reduced pressure. The resulting crude product was purified and prepared by silica gel plate (ethyl acetate/petroleum ether (v/v)=1/4) to obtain yellow solid 4-bromo-N-(4-methoxybenzyl)-N-methylpyridine-2-sulfonamide (500 mg, yield 84.5%) was obtained. ¹H NMR (400 MHz, CDCl₃) δ 8.45 (d, J=5.1 Hz, 1H), 8.06 (d, J=1.7 Hz, 1H), 7.58 (dd, J=5.1, 1.7 Hz, 1H), 7.23 (d, J=8.5 Hz, 1H), 6.85-6.76 (m, 3H), 4.30 (s, 2H), 3.74 (s, 3H), 2.74 (s, 3H). ESI-MS (m/z). 371.0 [M+H]⁺.

### Step c) Preparation of 2-(4,4-difluoroazepan-1-yl)-N-(2-(N-(4-methoxybenzyl)-N-methyl sulfamoyl)pyridin-4-yl) quinoline-3-carboxamide

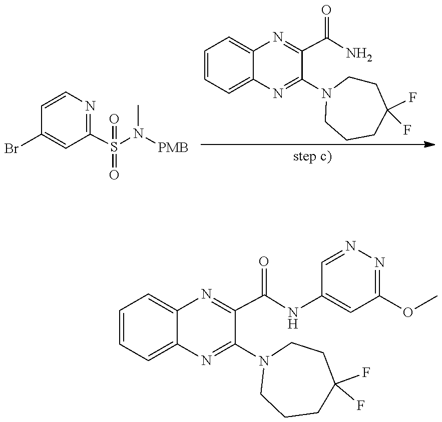

For the synthesis of 2-(4,4-difluoroazepan-1-yl) quinoline-3-carboxamide, see step a-e of Example 26.

4-Bromo-N-(4-methoxybenzyl)-N-methylpyridine-2-sulfonamide (250 mg, 0.67 mmol), 2-(4,4-difluoroazepan-1-yl) quinoline-3-carboxamide (205 mg, 0.67 mmol), Ruphos-Pd-G2 (52 mg, 0.067 mmol) and Cs₂CO₃ (768 mg, 2.36 mmol) were dissolved in toluene (5 mL) and stirred at 110° C. for 12 H under nitrogen protection. After the reaction was completed, 10 mL of water was added, then it was extracted with ethyl acetate (10 mL×2), and the combined organic phase was washed with saturated salt water (10 mL), dried with anhydrous sodium sulfate and filtered, and the filtrate was concentrated under reduced pressure. The crude product was purified by silica gel plate (petroleum ether/ethyl acetate (v/v)=2/3) and yellow solid 2-(4,4-difluoroazepan-1-yl)-N-(2-(N-(4-methoxybenzyl)-N-methylsulfamoyl)pyridin-4-yl) quinoline-3-carboxamide (370 mg, yield 92.1%) was obtained. ¹H NMR (400 MHz, DMSO-d₆) δ 11.46 (s, 1H), 8.75 (d, J=5.5 Hz, 1H), 8.50 (s, 1H), 8.40 (s, 1H), 8.03 (d, J=3.6 Hz, 1H), 7.91 (d, J=7.9 Hz, 1H), 7.74-7.69 (m, 2H), 7.42-7.36 (m, 1H), 7.34-7.29 (m, 2H), 7.01-6.97 (m, 2H), 4.36 (s, 2H), 3.85-3.76 (m, 6H), 3.66-3.58 (m, 2H), 2.78 (s, 2H), 2.51-2.40 (m, 2H), 2.12-2.01 (m, 2H), 2.00-1.91 (m, 2H). ESI-MS (m/z): 596.2 [M+H].⁺

### Step d) Preparation of 2-(4,4-difluoroazepan-1-yl)-N-(2-(N-methylsulfamoyl)pyridin-4-yl)quinoline-3-carboxamide

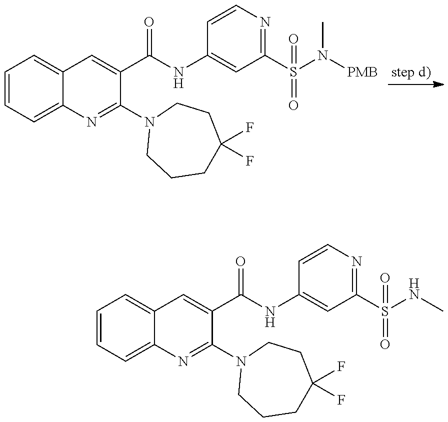

2-(4,4-Difluoroazepan-1-yl)-N-(2-(N-(4-methoxybenzyl)-N-methylsulfamoyl)pyridin-4-yl)quinoline-3-carboxamide (370 mg, 0.62 mmol) was dissolved in trifluoroacetic acid (5 mL) and trifluoromethanesulfonic acid (0.5 mL) was added and stirred at 70° C. for 2 h. After the reaction was completed, the reaction system was restored to room temperature, and it was then poured into ice water (10 mL), and extracted with 2-methyltetrahydrofuran (10 mL×2). The combined organic phase was washed with saturated salt water (10 mL), dried with anhydrous sodium sulfate and filtered, and the filtrate was concentrated under reduced pressure. The resulting crude product was prepared and purified to obtain light yellow solid powder 2-(4,4-difluoroazepan-1-yl)-N-(2-(N-methylsulfamoyl)pyridin-4-yl) quinoline-3-carboxamide (105.74 mg, yield 35.8%). ¹H NMR (400 MHz, DMSO-d₆) δ 11.36 (s, 1H), 8.65 (d, J=5.5 Hz, 1H), 8.44 (s, 1H), 8.32 (s, 1H), 7.95-7.90 (m, 1H), 7.85 (d, J=7.9 Hz, 1H), 7.75-7.69 (m, 1H), 7.67 (d, J=3.8 Hz, 2H), 7.37-7.30 (m, 1H), 3.78-3.71 (m, 2H), 3.56 (t, J=5.8 Hz, 2H), 2.57 (3H, d, J=4.8 Hz), 2.46-2.35 (m, 2H), 2.06-1.94 (m, 2H), 1.93-1.85 (m, 2H). ESI-MS (m/z): 476.1 [M+H]⁺.

### Example 29

### Preparation of 2-(4,4-difluoroazepan-1-yl)-6,7-difluoro-N-(2-(N-methylsulfamoyl)pyridin-4-yl)quinoline-3-carboxamide (KH29)

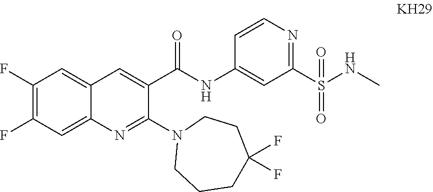 [SMILES: CNS(=O)(=O)c1cc(NC(=O)c2cc3cc(F)c(F)cc3nc2N2CCCC(F)(F)CC2)ccn1]

The synthesis of 2-(4,4-difluoroazepan-1-yl)-6,7-difluoro-N-(2-(N-methylsulfamoyl)pyridin-4-yl)quinoline-3-carboxamide is similar to example 28, except that 2-(4,4-difluoroazepan-1-yl)quinoline-3-carboxamide is replaced by 2-(4,4-difluoroazepan-1-yl)-6,7-difluoroquinoline-3-carboxamide (for the synthesis see step a-d of Example 10 and step e of Example 26), obtaining a quick light yellow solid 2-(4,4-difluoroazepan-1-yl)-6,7-difluoro-N-(2-(N-methylsulfamoyl)pyridin-4-yl)quinoline-3-carboxamide (61.82 mg, 23.13% yield). ¹H NMR (400 MHz, DMSO-d₆) δ 11.40 (s, 1H), 8.66 (d, J=5.5 Hz, 1H), 8.42 (s, 1H), 8.30 (d, J=1.9 Hz, 1H), 7.95-7.88 (m, 2H), 7.73 (q, J=4.9 Hz, 1H), 7.63-7.58 (m, 1H), 3.76-3.67 (m, 2H), 3.54 (2H, t, J=5.8 Hz), 2.56 (d, J=4.9 Hz, 3H), 2.47-2.33 (m, 2H), 2.07-1.93 (m, 2H), 1.90-1.84 (m, 2H). ESI-MS (m/z): 512.1 [M+H]⁺.

### Example 30

### Preparation of 2-(2-chloro-4-fluorophenoxy)-N-(3-sulfamoylphenyl)quinoline-3-carboxamide (Compound in Example 66 of Patent WO2014120815A1)

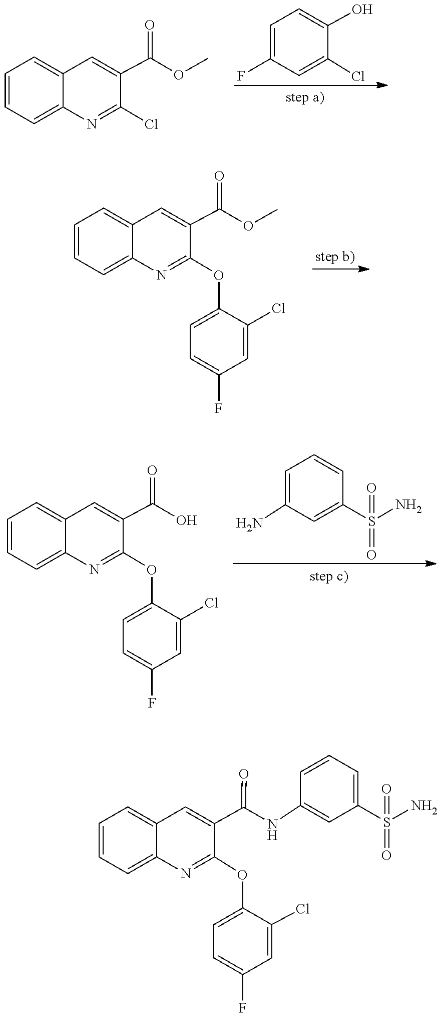

### Step a): Preparation of methyl 2-(2-chloro-4-fluorophenoxy)quinoline-3-carboxylate

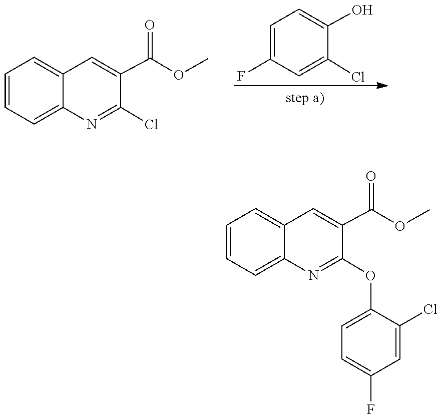

Methyl 2-chloroquinolin-3-carboxylate (0.117 g, 0.53 mmol), cesium carbonate (0.525 g, 1.6 mmol), 10 mL DMF and 2-chloro-4-fluorophenol (0.159 g, 1.1 mmol) were added into a single-neck bottle of 100 mL, and the reaction system was replaced with nitrogen for three times, and temperature was raised to 100° C. to continue the reaction. After the reaction was confirmed by TLC point plate, the system was cooled to room temperature. Then 50 mL pure water was added, and ethyl acetate (30 mL×3) was used for extraction, and the combined organic phase was washed with saturated sodium chloride, dried with anhydrous sodium sulfate and filtered, and the filtrate was concentrated to be dry with vacuum rotary evaporation. The crude product was purified by thin layer chromatography silica gel preparation plate (development agent: petroleum ether/ethyl acetate (v/v)=4/1; Eluent: dichloromethane/methanol (v/v)=10/1) and yellow solid methyl 2-(2-chloro-4-fluorophenoxy)quinoline-3-carboxylate (0.132 g, yield 75.5%) was obtained. ESI-MS (m/z): 332.4 [M+H]⁺.

### Step b): Preparation of 2-(2-chloro-4-fluorophenoxy)quinoline-3-carboxylic acid

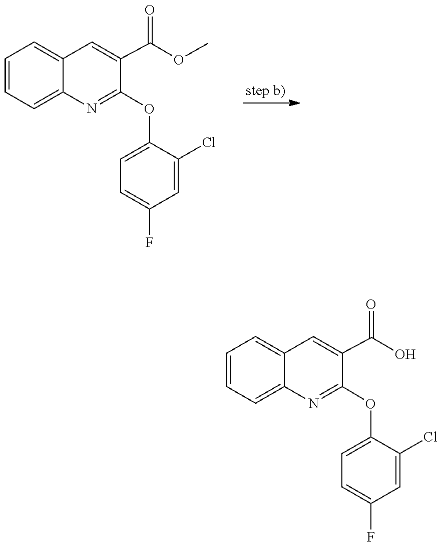 [SMILES: COC(=O)c1cc2ccccc2nc1Oc1ccc(F)cc1Cl ; O=C(O)c1cc2ccccc2nc1Oc1ccc(F)cc1Cl]

Methyl 2-(2-chloro-4-fluorophenoxy)quinoline-3-carboxylate (0.122 g, 0.37 mmol) and 4 mL methanol were added into a 100 mL single-neck bottle, and stirred for 10 minutes, then 20 mL pure water and lithium hydroxide (36 mg, 1.5 mmol) were added, and the system continued to react at 45° C. for 10 hours. The reaction mixture was concentrated to be dry under reduced pressure, then 20 mL pure water was added, and ethyl acetate (20 mL×2) was used for extraction. The water phase was adjusted pH4˜5 with 1M hydrochloric acid, then extracted with ethyl acetate (20 mL×3), and the combined organic phase was washed with saturated sodium chloride solution, dried with anhydrous sodium sulfate and filtered, and the filtrate was concentrated under reduced pressure to obtain white solid 2-(2-chloro-4-fluorophenoxy)quinoline-3-carboxylic acid (0.102 g, yield 87.3%). ESI-MS (m/z): 316.0 [M−H]⁻.

### Step c): Preparation of 2-(2-chloro-4-fluorophenoxy)-N-(3-sulfamoylphenyl)quinoline-3-carboxamide

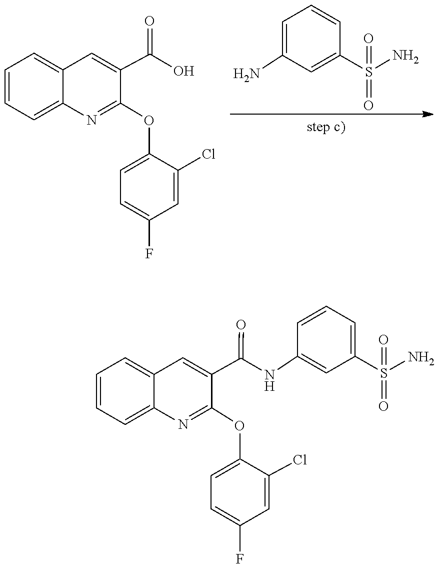 [SMILES: O=C(O)c1cc2ccccc2nc1Oc1ccc(F)cc1Cl ; Nc1cccc(S(N)(=O)=O)c1 ; NS(=O)(=O)c1cccc(NC(=O)c2cc3ccccc3nc2Oc2ccc(F)cc2Cl)c1]

2-(2-Chloro-4-fluorophenoxy)quinoline-3-carboxylic acid (60 mg, 0.189 mmol), 3-aminobenzenesulfonamide (39 mg, 0.226 mmol) and 4 mL DMF were added into a 250 mL three-neck bottle, and the reaction system was replaced with nitrogen for three times to protect it. After cooling in ice water bath for 20 minutes, DIPEA (53 mg, 0.41 mmol) was added, and then the solution of propylphosphonic anhydride (0.331 g, 0.52 mmol) was added by drops, and the reaction was continued for 1 h after dropping, and then it was raised to room temperature and stirred overnight. After the reaction was completed, 30 mL of pure water was added, and then it was extracted with ethyl acetate (30 mL×3), and the combined organic phase was washed with saturated sodium chloride aqueous solution, dried with anhydrous sodium sulfate and filtered, and the filtrate was concentrated to be dry under reduced pressure. The crude product was purified by thin layer chromatography silica gel preparation plate (development agent: dichloromethane/methanol (v/v)=10/1); Eluent: dichloromethane/methanol (v/v)=10/1) and white solid 2-(2-chloro-4-fluorophenoxy)-N-(3-sulfamoylphenyl)quinoline-3-carboxamide (17 mg, yield 19.06%) was obtained. ¹H NMR (400 MHz, DMSO-d₆) δ 10.94 (s, 1H), 8.79 (s, 1H), 8.39 (s, 1H), 8.09 (d, J=8.0 Hz, 1H), 7.95-7.82 (m, 1H), 7.79-7.71 (m, 1H), 7.68-7.54 (m, 6H), 7.48-7.32 (m, 3H). ESI-MS (m/z): 472.2 [M+H]⁺.

### Example 31

### Preparation of 5-chloro-2-(4,4-difluoroazepan-1-yl)-N-(2-sulfamoylpyridin-4-yl)-4-trifluoromethylbenzamide (Compound A6 in Patent CN112457294B)

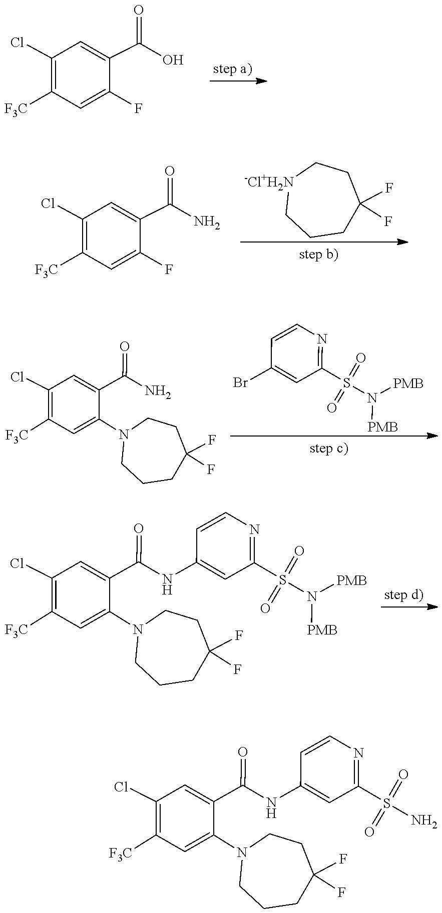

### Step a): Preparation of 5-chloro-2-fluoro-4-trifluoromethylbenzamide

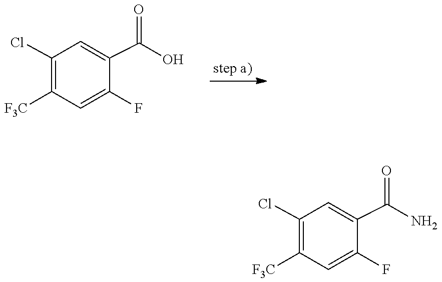 [SMILES: O=C(O)c1cc(Cl)c(C(F)(F)F)cc1F ; NC(=O)c1cc(Cl)c(C(F)(F)F)cc1F]

Oxalyl chloride (25 mL) and DMF (400 mg, 5.47 mmol) were added to a solution of 5-chloro-2-fluoro-4-trifluoromethylbenzoic acid (22 g, 90 mol) in dichloromethane (100 mL) at 0° C. and it was continually stirred for 0.5 h. After the reaction was completed, the reaction solution was concentrated under reduced pressure to obtain yellow oil, and the dichloromethane was added (100 mL), and the system was placed in an ice water bath, then ammonia water (50 mL) was slowly added, and stirred for 1 H. After the reaction was completed, the reaction system was diluted with water (100 mL), extracted with dichloromethane (100 mL×2), and the combined organic phase was washed with saturated salt water (50 mL), dried with anhydrous sulfuric acid and filtered. The filtrate was concentrated under reduced pressure to obtain white solid 5-chloro-2-fluoro-4-trifluoromethylbenzamide (21 g, yield 95%). ¹H NMR (400 MHz, DMSO-d₆) δ 8.06 (s, 1H), 7.96 (s, 1H), 7.93 (s, 1H), 7.91 (d, J=2.5 Hz, 1H). ESI-MS (m/z): 239.7 [M−H]⁻.

### Step b): Preparation of 5-chloro-2-(4,4-difluoroazepan-1-yl)-4-trifluoromethylbenzamide

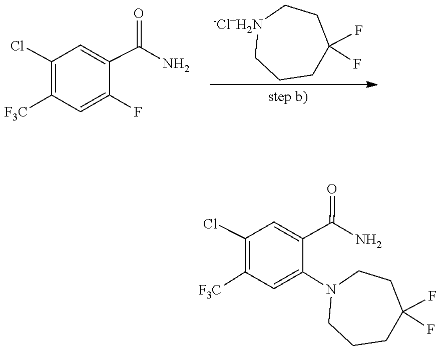

4,4-Difluoroazepane hydrochloride (30.6 g, 0.18 mol) and N,N-diisopropylethylamine (34.8 g, 0.27 mol) were added to a solution of 5-chloro-2-fluoro-4-trifluoromethylbenzamide (21.7 g, 0.09 mol) in N-methylpyrrolidone (200 mL) and stirred at 100° C. for 16 h. After the reaction was completed, the reaction system was quenched with water (600 mL), then extracted with ethyl acetate (400 mL×2), and the combined organic phase was washed with saturated salt water (200 mL), dried with anhydrous sulfuric acid and filtered. The filtrate was concentrated under reduced pressure, and the crude product was purified by silica gel column chromatography (ethyl acetate/petroleum ether (v/v)=1/3) to obtain yellow solid 5-chloro-2-(4,4-difluoroazepan-1-yl)-4-trifluoromethylbenzamide (27 g, yield 84%). ¹H NMR (400 MHz, DMSO-d₆) δ 8.06 (s, 1H), 7.69 (s, 1H), 7.49 (s, 1H), 7.27 (s, 1H), 3.44-3.38 (m, 2H), (3.35-3.30 m, 2H), 2.31-2.22 (m, 2H), 2.17-2.05 (m, 2H), 1.89-1.80 (m, 2H). ESI-MS (m/z): 357.0 [M+H]⁺.

### Step c): Preparation of N-(2-(N,N-bis(4-methoxybenzyl)sulfamoyl)pyridin-4-yl)-5-chloro-2-(4,4-difluoroazepan-1-yl)-4-trifluoromethylbenzamide

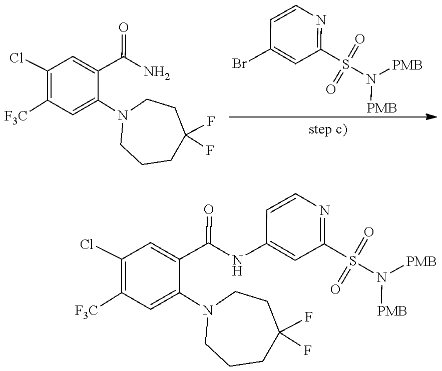

4-Bromo-N,N-bis(4-methoxybenzyl)pyridine-2-sulfonamide (16 g, 33.5 mmol), Pd₂(dba)₃ (2.3 g, 2.5 mmol), BINAP (3.14 g, 5.0 mmol) and cesium carbonate (27.3 g, 84.0 mmol) were added to a solution of 5-chloro-2-(4,4-difluoroazepan-1-yl)-4-trifluoromethylbenzamide (12 g, 33.5 mmol) in toluene (100 mL) and stirred at 85° C. for 16 h. After the reaction was completed, the reaction system was diluted with water (300 mL), extracted with ethyl acetate (300 mL×2), and the combined organic phase was washed with saturated salt water (100 mL), dried with anhydrous sodium sulfate and filtered. The filtrate was concentrated under reduced pressure. The crude product was purified by silica gel column chromatography (ethyl acetate/petroleum ether (v/v)=1/5) to obtain a yellow solid N-(2-(N,N-bis(4-methoxybenzyl)sulfamoyl)pyridin-4-yl)-5-chloro-2-(4,4-difluoroazepan-1-yl)-4-trifluoromethylbenzamide (20 g, yield: 79%). ESI-MS (m/z): 753.2 [M+H]⁺.

### Step d): Preparation of 5-chloro-2-(4,4-difluoroazepan-1-yl)-N-(2-sulfamoylpyridin-4-yl)-4-trifluoromethylbenzamide

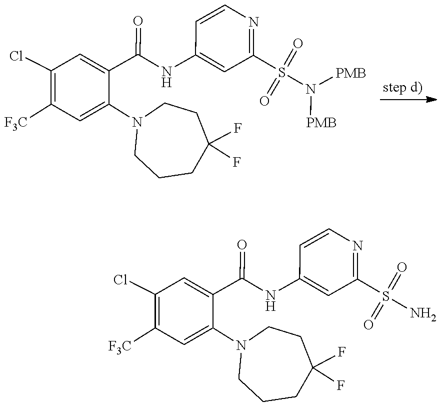

Trifluoromethanesulfonic acid (1 mL, 7.1 mmol) was slowly added to a trifluoroacetic acid (13 mL) solution of N-(2-(N,N-bis(4-methoxybenzyl)sulfamoyl)pyridin-4-yl)-5-chloro-2-(4,4-difluoroazepan-1-yl)-4-trifluoromethylbenzamide (7 g, 9.29 mmol) and reacted at 50° C. for 0.5 h. After the reaction was completed, the reaction solution was slowly added to ice water (100 mL), and stirred for 0.5 h, and filtered to obtain crude product. The crude product was purified by C18 reverse column (trifluoroacetic acid system) to obtain white solid (2.3 g, yield 48%). ¹H NMR (400 MHz, DMSO-d₆) δ 11.21 (s, 1H), 8.63 (d, J=5.2 Hz, 1H), 8.31 (s, 1H), 7.88-7.80 (m, 1H), 7.76 (s, 1H), 7.47 (s, 2H), 7.36 (s, 1H), 3.41-3.39 (m, 1H), 3.36-3.34 (m, 1H), 2.29-2.14 (m, 2H), 2.12-1.98 (m, 2H), 1.83-1.74 (m, 2H). ESI-MS (m/z): 513.2 [M+H]⁺.

### Example 32

### Preparation of 2-(4-fluoro-2-methylphenoxy)-N-(2-carbonyl-1,2-dihydropyridin-4-yl)-4-(trifluoromethyl)benzamide (Parent Drug of VX150)

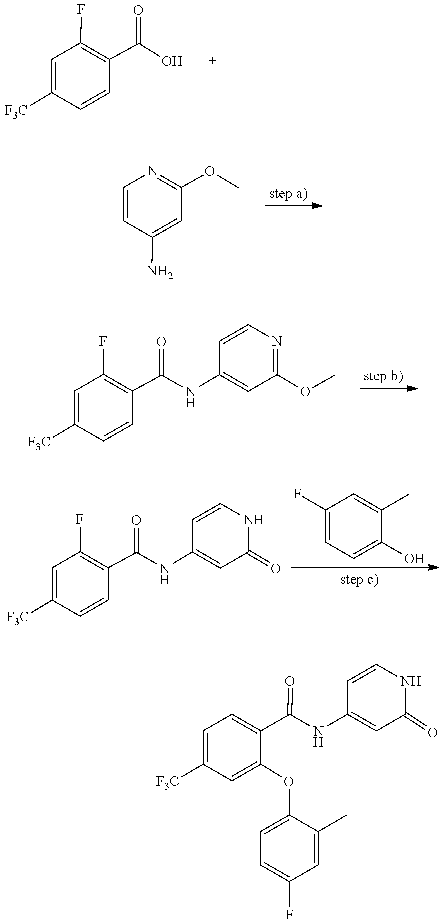

### Step a): Preparation of 2-fluoro-N-(2-methoxypyridin-4-yl)-4-(trifluoromethyl)benzamide

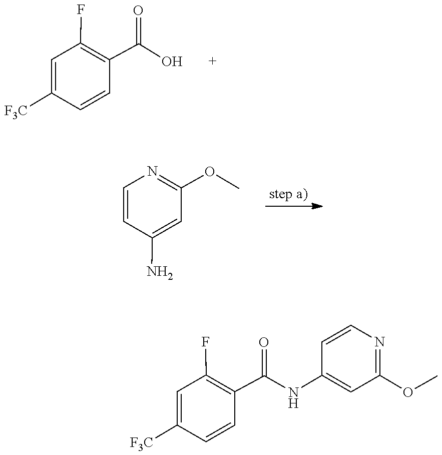

Triethylamine (1.7 g, 16.8 mmol) was added to dichloromethane (30 mL) solution of 2-fluoro-4-trifluoromethyl benzoic acid (1 g, 4.8 mmol), 2-methoxy-4-aminopyridine (0.626 g, 5 mmol) and HATU (2.741 g, 7.2 mmol) under nitrogen protection and stirred at room temperature for 16 h. After the reaction was completed, 50 mL pure water was added and stirred evenly, the liquid was divided and the lower organic phase was kept for use, and the upper aqueous phase was extracted with dichloromethane (30 mL×3), all organic phases were combined, and then washed with saturated NaCl solution (30 mL×2), dried with anhydrous sodium sulfate and filtered, and the filtrate was concentrated to be dry with vacuum rotary evaporation. The crude product was purified by silica gel column chromatography (PE/EA (v/v)=8/1-3/1) to obtain white solid 2-fluoro-N-(2-methoxypyridin-4-yl)-4-(trifluoromethyl) benzamide (1.179 g, yield 78.1%).

### Step b): Preparation of 2-fluoro-N-(2-carbonyl-1,2-dihydropyridin-4-yl)-4-(trifluoromethyl) benzamide

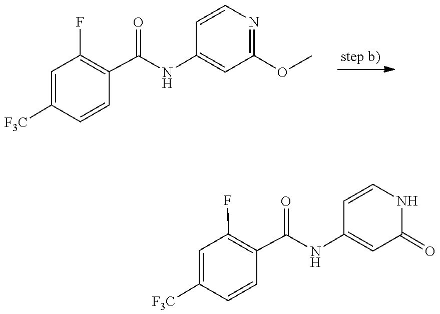 [SMILES: COc1cc(NC(=O)c2ccc(C(F)(F)F)cc2F)ccn1 ; O=C(Nc1cc[nH]c(=O)c1)c1ccc(C(F)(F)F)cc1F]

Hydrobromic acid/acetic acid solution (10 mL, 33%) was added to 2-fluoro-N-(2-methoxy-pyridin-4-yl)-4-(trifluoromethyl) benzamide (0.5 g, 1.6 mmol) under the protection of nitrogen. The solution was reacted at 100° C. for 5 h, and then at room temperature for 12 h. 40 mL of pure water was added and stirred for 30 min, then filtered, and the filter cake was washed with pure water (10 mL×2), and dried at 50° C. under reduced pressure to obtain gray solid 2-fluoro-N-(2-carbonyl-1,2-dihydropyridin-4-yl)-4-(trifluoromethyl) benzamide (0.215 g, yield 45%).

### Step c): Preparation of 2-(4-fluoro-2-methylphenoxy)-N-(2-carbonyl-1,2-dihydropyridin-4-yl)-4-(trifluoromethyl) benzamide

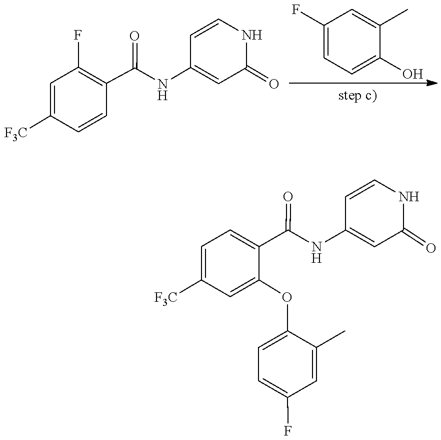

DMF (6 mL) was added to the mixture of 2-fluoro-N-(2-carbonyl-1,2-dihydropyridin-4-yl)-4-(trifluoromethyl) benzamide (0.21 g, 0.7 mmol), 4-fluoro-2-methylphenol (0.261 g, 2 mmol) and cesium carbonate (0.684 g, 2 mmol) under nitrogen protection and reacted at 80° C. for 2 H. After the reaction was completed, the system was cooled to room temperature, and 50 mL of pure water was added, and stirred for 30 min, then it was filtered, and the filter cake was washed with pure water (20 mL×2) and petroleum ether (20 mL×2) in turn, and then dried under reduced pressure to obtain 0.278 g crude product. The crude product was completely dissolved with ethyl acetate (about 5 mL) and stirred for 5 min, then petroleum ether (about 15 mL) was slowly added, and a large amount of precipitation was precipitated, and it continued to be stirred for 2 h and filtered. The filter cake was washed with petroleum ether (10 mL×2) and dried under reduced pressure to obtain white solid 2-(4-fluoro-2-methylphenoxy)-N-(2-carbonyl-1,2-dihydropyridin-4-yl)-4-(trifluoromethyl) benzamide (0.212 g, yield 75.6%). ¹H NMR (400 MHz, DMSO-d₆) δ 11.29 (s, 1H), 10.64 (s, 1H), 7.84 (d, J=7.9 Hz, 1H), 7.60 (d, J=8.5 Hz, 1H), 7.31 (d, J=7.2 Hz, 1H), 7.27-7.20 (m, 1H), 7.10 (dd, J=6.7, 3.1 Hz, 1H), 6.97 (s, 1H), 6.75 (s, 1H), 6.38 (dd, J=7.2, 2.0 Hz, 1H), 2.16 (s, 3H). ESI-MS (m/z): 407.2 [M+H]⁺.

### Example 33: Electrophysiological Assay

The patch clamp technique is known as the “gold standard” for studying ion channels, and it uses glass microelectrodes to seal the cell membrane to measure the currents of various membrane channel. Navs are a class of transmembrane proteins, which contain three different states, the resting state, the active state and the inactive state. Patch clamp technology can evaluate the inhibition strength of different compounds on different states of Navs by measuring the changes of membrane channel currents after different compounds bind to different states of Navs.

The representative compounds of the present invention used stable CHO or HEK293 cell lines transfected with specific ion channels to measure the effect of the compounds on Nav1.8 currents by manual patch clamp test, to evaluate their inhibition strength.

### Method 1:

The assay protocol of manual patch clamp is as follows:

### 1) Cell Culture

CHO cell lines with stable expression of hNav1.8 were cultured in F12 medium containing 10% fetal bovine serum at a constant temperature of 37° C., and the carbon dioxide concentration was maintained at 5%. The old culture medium was removed and washed once with PBS, then 1 mL 0.25%-Trypsin-EDTA solution was added and incubated at 37° C. for 1 min. When the cells were detached from the bottom of the dish, 5 mL of the complete medium preheated at 37° C. was added, and the aggregated cells were separated by gently blowing with a straw. The cell suspension was transferred into a sterile centrifuge tube and centrifuged at 1000 rpm for 5 min to collect cells. The cells were then inoculated into a cell culture dish with a diameter of 6 cm (2.5×10⁵ cells/petri dish, 5 mL medium) for expansion or maintenance.

In order to maintain the electrophysiological activity of the cells, the cell density must be less than 80%.

The cells were separated with 0.25%-Trypsin-EDTA before the assay, and 5×10³ cells were seeded on a cover slip and cultured in a 24-well plate (final volume: 500 μL) for 18 h. Then the assay was conducted.

### 2) Preparation of Compound Samples

The compounds prepared in the examples of the present invention were dissolved in dimethyl sulfoxide (DMSO) and prepared into DMSO stock solution with a concentration of 10 mM for assay. And it was diluted to various concentrations with extracellular solution (140 mM NaCl, 3.5 mM KCl, 1 mM MgCl₂, 2 mM CaCl₂), 10 mM Glucose, 10 mM HEPES, 1.25 mM NaH₂PO₄, pH=7.4 regulated by NaOH), and the final concentration of DMSO in each compound sample was 0.10% or less.

### 3) Patch Clamp Measures Sodium Channel Blocking Effect

A capillary glass pipette (BF150-86-10, Sutter Instruments) was pulled into a recording electrode with a micropipette puller (P97, Sutter Instruments). The micromanipulator (MP285, Sutter Instruments) was manipulated under an inverted microscope (IX71, Olympus) to make the recording electrode contact the cell and negative pressure suction was applied to achieve a GQ seal. Then, rapid capacitance compensation was performed, and negative pressure was continued, cell membrane was broken by suction, and whole cell recording mode was formed. Then the slow capacitance was compensated and the membrane capacitance and series resistance were recorded without leakage compensation. When the whole cell recording current was stabilized, dosing was initiated, and the next concentration was detected after acting 5 minutes of each drug concentration. Multiple cells were independently repeated during the recording period. All electrophysiological assays were performed at room temperature. Specifically, 6 concentrations (measuring IC₅₀) or 2 concentrations (preliminary screening) were set for each compound, and the percentage of inhibition of sodium channels was determined by calculating the relative percentage of peak current generated before and after each concentration compound treated cells, and the IC₅₀ value or percentage of inhibition at a specific concentration was calculated using IGOR pro software.

The voltage stimulation protocol for recording hNav1.8 sodium channel current using whole cell patch clamp is as follows: the membrane potential is clamped at −120 mV, the command voltage starts from −130 mV, and is maintained in steps of 10 mV for 8 seconds, then depolarized to 0 mV (or current size is 0 pA), maintained for 30 ms, and the half inactivation voltage is measured. After the formation of whole cell sealing, the cell membrane voltage is clamped at −120 mV, and the clamp voltage is depolarized to 0 mV for 50 ms, then the voltage is restored to the measured half inactivation voltage for 8 s. The cell membrane potential then recovers to −120 mV for 20 ms, then depolarized to 0 mV for 50 ms, and finally restored to the clamp voltage of −120 mV for 30 ms. The data were collected repeatedly every 20 s to observe the effect of the drug on the peak current of hNav1.8 sodium channel.

The inhibitory activity (resting state) of some examples on hNav1.8 was calculated as shown in Table 2 below.

TABLE 2
Inhibitory activity of the examples against hNav1.8

| No. | IC₅₀ (nM) |
|---|---|
| Example 01 | >100 |
| Example 02 | 5.3 |
| Example 03 | >10 |
| Example 04 | 66.7 |
| Example 05 | 8.9 |
| Example 06 | 3.1 |
| Example 07 | 14.7 |
| Example 08 | 3.3 |
| Example 09 | 1 |
| Example 10 | 0.784 |
| Example 14 | 0.34 |
| Example 16 | 0.084 |
| Example 17 | 0.095 |
| Example 21 | 0.36 |
| Example 32 | 16.0 |

TABLE 3
Inhibition rates of hNav1.8 for example compounds

| No. | 10 nM | 100 nM |
|---|---|---|
| Example 01 | 0.36% | 41.79% |
| Example 02 | 66.12% | 96.07% |
| Example 03 | 19.72% | 70.03% |
| Example 04 | 3.33% | 45.95% |
| Example 05 | 46.68% | 90.46% |
| Example 06 | 77.57% | NT |
| Example 07 | 48.30% | NT |
| Example 08 | 75.39% | 96.61% |
| Example 09 | 91.28% | NT |
| Example 10 | 95.46% | 99.08% |
| Example 11 | 15.27% | 72.75% |
| Example 12 | 95.79% | 99.96% |
| Example 13 | 95.64% | 99.65% |
| Example 14 | 98.02% | 99.63% |
| Example 15 | 15.38% | 49.93% |
| Example 16 | 99.83% | 99.98% |
| Example 17 | 98.28% | 100% |
| Example 18 | −3.14% | 21.54% |
| Example 19 | 0.44% | 16.56% |
| Example 20 | 19.48% | 81.11% |
| Example 22 | / | 90.7% |
| Example 23 | / | 97.18% |
| Example 24 | / | 50.96% |
| Example 25 | / | 91.55% |
| Example 26 | / | 91.49% |
| Example 27 | / | 97.02% |
| Example 28 | / | 94.24% |
| Example 29 | / | 83.67% |
| Example 30 | / | 94.00% |
| Example 31 | / | 97.07% |
| Example 32 | 38.12% | 84.71% |

### Method 2:

Human Nav1.8 ion channel is stably expressed on HEK293 cells. After the Nav1.8 current is stabilized, the effect of the compounds on Nav1.8 ion channel can be obtained by comparing the NaV1.8 current before and after the compounds are applied.

### 1) Formulation

Reagents except NaOH and KOH which are used for acid-base titration, are all purchased from Sigma (St. Louis, MO) Company. The test compounds were accurately weighed and then prepared into a solution in DMSO with a concentration of 9 mM. The final concentrations were all prepared on the day of the assay and then dissolved in the extracellular solution. Extracellular solution (mM) was: NaCl, 137; KCl, 4; CaCl₂), 1.8; MgCl2, 1; HEPES, 10; glucose 10; pH 7.4 (NaOH titration). All test compounds and control compound solutions contained 1 μM TTX.

### 2) Assay Method

All assays were conducted at room temperature, with a control of each cell itself. The compounds were all irrigated by a perfusion system that utilized its own gravity. At least two cells were tested for each concentration. After the current had been stabilized (or 5 min), the strength of the current change before and after applying compounds were compared and used to calculate the blocking effect of the compound.

### a) Assay Instruments

Patch clamp amplifier: Patch Clamp PC-505B (WARNER instruments)/MultiClamp 700A (Axon instrument)
Digital-to-analog Converter: Digidata 1440A (Axon CNS)/Digidata 1550A (Axon instruments)
Micromanipulator: MP-225 (SUTTER instrument)
Inverted microscope: TL4 (Olympus)
Glass micropipette puller: PC-10 (NARISHIGE)
Microelectrode glass capillary: B12024F (Wuhan Microtanyi Scientific Instrument Co., LTD.)

### b) Electrophysiology

The cells were transferred to a perfusion tank for perfusion with extracellular fluid. Intracellular fluid (mM): Aspartic acid, 140; MgCl₂, 2; EGTA 11; HEPES, 10; pH 7.2 (CsOH titration). Intracellular solution was stored at −80° C. refrigerator in small batches and melt on test day. The electrodes were drawn with PC-10 (Narishige, Japan). During the whole-cell patch clamp recording, noise was filtered at one-fifth of the sampling frequency.

### c) Assay Protocol (Resting State)

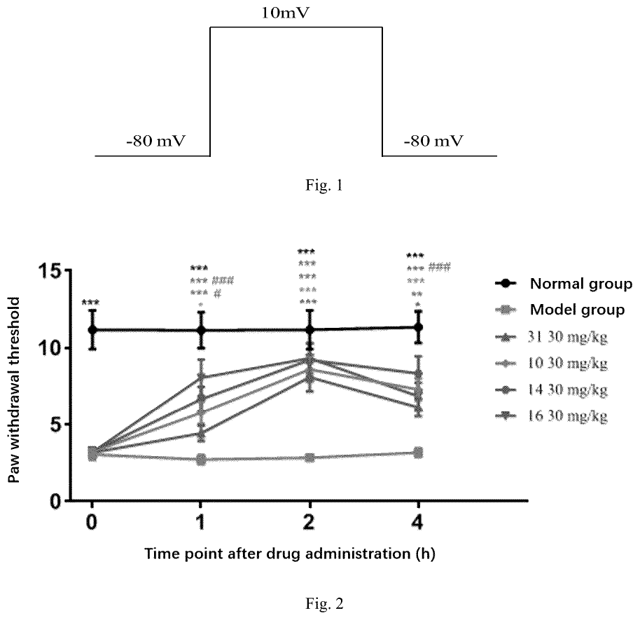

Clamped the cell at −80 mV and then depolarized to 10 mV with a square wave lasting 10 ms to get the Nav1.8 current (see FIG. 1 attached). This procedure was repeated every 5 seconds. The maximum current triggered by the square wave was detected, and when it stabilized, the test compounds were perfused, and when the reaction stabilized, the strength of the block was calculated.

The inhibitory activity of some examples on hNav1.8 was calculated as shown in Table 4 below.

TABLE 4
The inhibitory activity of the examples against hNav1.8

| No. | IC₅₀ (nM) |
|---|---|
| Example 02 | 2.82 |
| Example 05 | 21.3 |
| Example 08 | 1.74 |
| Example 09 | 0.918 |
| Example 10 | 0.46 |
| Example 14 | 0.725 |
| Example 16 | 0.071 |
| Example 23 | 1.5 |
| Example 24 | 97.11 |
| Example 25 | 9.23 |
| Example 26 | 8 |
| Example 28 | 11.42 |
| Example 30 | 9.5 |
| Example 32 | 35.1 |

### Example 34: Assay of Kinetic Solubility

### 1) Preparation of Stock Solutions

The stock solutions of the test compound and the standard progesterone were prepared in DMSO at the concentration of 10 mM.

### 2) Assay Procedures for Solubility

30 μL of stock solutions at 10 mM of the test compounds and standard substance were placed in order into their proper 96-well plate. 970 μL phosphate buffer at the specified pH was added to the proper vial of the sample plate. The assay was performed in duplicate. A stirring stick was added to each vial and the teflon/silicone plug was capped. The sample tray was then placed in the Eppendorf Thermomixer Comfort and shaken at 1100 RPM at 25° C. for 2 hours. After 2 hours, plugs were removed, a big magnet was used to remove the stirring sticks, and samples were transferred from the sample plate to the filter plate. Vacuum pump was used to create negative pressure and filtered the samples. Transferred 5 μL of the filtrate to the new sample plate, then added 5 μL DMSO and 490 μL water/acetonitrile (V/V=1/1) to dilute the solvent. The dilution factor may be adjusted according to the solubility values of the substances to be tested or the strength of its LC-MS signal response.

### 3) Preparation of Standard Solution

From the 10 mM DMSO stock solution, 15 μL of that was transferred into the empty plate and 485 μL DMSO was added to prepare a 300 μM standard solution. From the 300 μM standard solution plate, 5 μL of that was transferred to another empty plate and 5 μL phosphate buffer at specified pH and 490 μL water/acetonitrile (V/V=1/1) dilution solvent were added to the standard solution to get a final concentration of 3 μM. The concentration of the standard solution may be adjusted according to the strength of its LC-MS signal response.

### 4) Sample Analysis Steps

The sample plate was placed into the sample tray of the automatic sampler and the samples were evaluated by LC-MS/MS analysis.

### 5) Data Analysis

All calculations were carried out by Microsoft Excel.

The sample filtrate was analyzed and quantified against a standard of known concentrations using LC-MS spectral peaks to identify and quantify. The solubility values of the control drug and the compounds to be tested were calculated as the following formula:
[Sample]: concentration of the sample
AREA_{Sample}: peak area of the sample
INJ VOL_{Std}: standard sample injection volume
DF_{Sample}: sample dilution factor
[STD]: standard substance concentration
AREA_{Std}: peak area of standard substance
INJ VOL_{Sample}: sample injection volume

TABLE 5
Kinetic solubility of example compounds (unit:
μM) (compounds with comparable activity)

| No. | pH 3.5 |
|---|---|
| Example 02 | 2.80 |
| Example 05 | 0.39 |
| Example 06 | 0.39 |
| Example 07 | 52.7 |
| Example 08 | 175.0 |
| Example 09 | 157.21 |
| Example 13 | 3.38 |
| Example 14 | 270.67 |
| Example 24 | 9.88 |
| Example 25 | 87.93 |
| Example 26 | 127 |
| Example 27 | 78.8 |
| Example 28 | 131 |
| Example 29 | <1.56 |
| Example 30 | <0.18 |
| Example 31 | 0.69 |
| Example 32 | <1.56 |

### Example 35: Stability Assay Method for Human Liver Microsomes In Vitro

### 1. Materials

Human liver microsomes, purchased from Corning or Xenotech, stored in a −80° C. refrigerator.
Nicotinamide adenine dinucleotide phosphate (NADPH), Chem-impex international, 00616
Control compounds: testosterone, diclofenac, propafenone

### 2. Instruments

Water bath (HH-60, Guohua, Changzhou, China)
Pipette workstation (PP-550DS, Apricot, Covina, USA)
Centrifuge (5810R, Eppendorf, Hamburg, Germany)
Mass Spectrometer (Sciex API 4000, Singapore)
High Performance Liquid Chromatography (LC 20-AD, Shimadzu, Kyoto, Japan)
Sampler (ADDA, Apricot Designs, California, USA)

### 3. Assay Procedures

### 3.1 Preparation of Working Solution

Stock solution: 10 mM in DMSO
Working solution concentration preparation: 10 mM stock solution was diluted to 100 μM with 100% acetonitrile (organic phase content: 99% ACN, 1% DMSO)

### 3.2 Assay Procedure

Two 96-well incubation plates were named T60 incubation plate and NCF60 incubation plate.

445 μL microsomal working solution (liver microsomal protein concentration: 0.56 mg/mL) was added to the T60 and NCF60 incubation plates respectively, then the incubation plates above were placed in a water bath at 37° C. for pre-incubation approximately 10 minutes.

After the pre-incubation finished, 5 μL of the test compound working solution was added to the T60 and NCF60 incubation plates respectively, and mixed well. 50 μL potassium phosphate buffer was added to each well on the NCF60 incubation plate to start the reaction. 180 μL termination solution (acetonitrile solution containing 200 ng/mL tolbutamide and 200 ng/mL labetalol) and 6 uL working solution of NADPH regeneration system were added to the TO termination plate, and 54 μL samples were removed from the T60 incubation plate to the TO termination plate (TO sample generated). The reaction was initiated by adding 44 μL of NADPH regenerating system working solution per well on the T60 incubation plate. Only 54 μL microsomal working solution, 6 uL NADPH regeneration system working solution, and 180 μL termination solution were added to the Blank plate. Therefore, in the samples of the test or control compounds, the final reaction concentration of the compounds, testosterone, diclofenac and propafenone was 1 μM, the concentration of the liver microsomes was 0.5 mg/mL, and the final concentration of DMSO and acetonitrile in the reaction system was 0.01% (v/v) and 0.99% (v/v), respectively.

After incubation for an appropriate time (e.g. 5, 10, 20, 30, and 60 minutes), 180 μL of termination solution (acetonitrile solution containing 200 ng/mL tolbutamide and 200 ng/mL labetalol) was added to the sample hole of each termination plate, respectively. After that, 60 μL sample was removed from the T60 incubation plate to terminate the reaction.

All sample plates were shaken evenly and centrifuged at 3220×g for 20 min. Then 80 μL supernatant was taken from each well and diluted to 240 μL pure water for LC-MS/MS analysis.

### 4. Liquid Chromatographic Tandem Mass Spectrometry Analysis

All samples were injected for analysis. The liver microsomal stability results were shown in Table 6.

TABLE 6
Human liver microsome stability (T_{1/2}) test results are as follows:

| No. | T_{1/2} (min) |
|---|---|
| Example 1 | >145 |
| Example 2 | 58.8 |
| Example 3 | 79.3 |
| Example 4 | 79.9 |
| Example 5 | 93.7 |
| Example 6 | >145 |
| Example 7 | 63.9 |
| Example 8 | 23 |
| Example 9 | 22.3 |
| Example 10 | 92.7 |
| Example 14 | >145 |
| Example 16 | 41.3 |
| Example 17 | >145 |
| Example 21 | 56.6 |
| Example 24 | >145 |
| Example 26 | 70 |
| Example 27 | 135.7 |

### Example 36 Efficacy Test in Postoperative Pain Model in Rats

### Experimental Animals

Sprague-Dawley male rats, purchased from Zhejiang Vital River Laboratory Animal Technology Co., LTD

### Sample Solution Preparation

Compounds of the example 10, 14, 16, 31 of the invention were weighted and prepared into solutions of 3 mg/mL using 5% DMSO (Anhui Senrise Technologies Co., Ltd)+30% Solutol HS15 (Beijing Ouhe Technology Co., LTD.)+65% water for injection, and administered at a volume of 10 mL/kg by gavage. The dosage was 30 mg/kg.

### Experimental Procedure

Animals were anesthetized with Zoletil 50+ Xylazine Hydrochloride (20 mg/kg+8 mg/kg, intraperitoneally injected). The surgical area on the plantar skin of the left hind paw was disinfected three times with iodophor and 70% ethanol, and the surgery was started after the skin was dry.

A longitudinal incision about 1 cm was made towards the toes starting from 0.5 cm away from the heel of the left hind paw. After the skin was incised, the flexor digitorum brevis was raised and longitudinal blunt injury was caused. After hemostasis by pressing, the wound was sutured. After surgery, animals were placed on an electric warm pad and 5 mL of physiological saline was injected subcutaneously to prevent dehydration. 24 h after modeling, all animals (including the normal control group) were tested for base mechanical allodynia (PWT). The modeling animals without mechanical hypersensitivity (PWT greater than 5 g) were excluded and 50 animals were selected and randomly divided into 5 groups, with 9 animals in each group except 5 in the normal group (negative control group).

The animals were weighed and administered orally at a volume of 10 mL/kg. Except normal control animals, other animals were given a single dose of compounds, and mechanical allodynia was performed by test fibers on all groups of animals at 1 h, 2 h and 4 h after dosing. The rats were placed individually in a plexiglass box with a grid at the bottom to ensure that the paws of the rats could be tested. The rats would acclimate for 15 minutes before the test. After the acclimation was completed, the test fibers were used to test on the plantar center of the rat's left hind paw. Pressed the test fiber vertically against the skin and applied force to bend the fiber for 6 to 8 seconds, with 5 second intervals between each test. The rapid withdrawal of the animal's paws during the test was recorded as a pain response. The animal's paw withdrawal when the fibers left the animal's skin was also recorded as a pain response. If the animal moves or walks around, it's not recorded as a pain response, and the test should be repeated.

### Method of Calculation and Data Processing

Mechanical hyperalgesia was expressed as the paw withdrawal threshold (PWT) in the rat behavioral test, which was calculated according to the following formula:

50% response threshold (g)=(10^{(Xf+kδ)}/10,000
Xf=final test fiber value used in the test
k=table value (Chaplan et al. 1994, page 62)
δ=mean deviation

Data were collected with excel software and analyzed with Prism 7.0 (Graph pad software, Inc.) software by two-way ANOVA followed by Dunnett multiple comparison test.

### Results

The result is shown in FIG. 2. In this experiment, example compound 31 inhibited mechanical hyperalgesia induced by postoperative pain models in rats at 2 h and 4 h after a single oral administration of 30 mg/kg. Example compound 10, 14, 16 inhibited mechanical hyperalgesia induced by postoperative pain models in rats at 1 h, 2 h, and 4 h after a single oral administration of 30 mg/kg. The efficacy of compound 14 was significantly stronger than that of example compound 31 at 1 h and 4 h after administration; and example compound 16 was significantly more effective than example compound 31 after 1 h of administration.

Note: *p<0.05, **p<0.01, ***p<0.001 compared with normal group; # p<0.05, ### p<0.001 compared with example compound 31.

1.-29. (canceled)

30. A compound of Formula (I) or a pharmaceutically acceptable salt thereof: 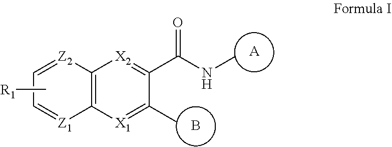
wherein:
X₁, X₂, Z₁, Z₂ are independently selected from substituted or unsubstituted C or N;
R₁ is independently selected from hydrogen, halogen, —NH₂, —CN, OH, —C₁-C₆ alkyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃; wherein R₂ and R₃ are independently selected from hydrogen, —NH₂, and —C₁-C₃ alkyl;
ring A is substituted or unsubstituted benzene ring or six-membered heteroaryl; and
ring B is the substituted or unsubstituted three- to ten-membered aliphatic ring or aliphatic heterocyclic ring.

31. The compound or the pharmaceutically acceptable salt of claim 30, wherein at least one of X₁ and X₂ is N.

32. The compound or the pharmaceutically acceptable salt of claim 30, wherein ring A is selected from a six-membered aryl or heteroaryl containing 0-3 nitrogen atoms, wherein the aryl or heteroaryl is optionally substituted by hydrogen, halogen, —NH₂, —CN, —OH, C₁-C₆ alkyl, carbonyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃ or —POR₂R₃; wherein R₁ and R₂ are independently selected from hydrogen, —NH₂, —NHCH₃, and —C₁-C₃ alkyl.

33. The compound or the pharmaceutically acceptable salt of claim 30, wherein ring B is selected from a 3-10 aliphatic ring or aliphatic heterocyclic ring with 0-3 heteroatoms which are selected from N, O, and S, and optionally, the aliphatic ring or aliphatic heterocyclic ring is substituted by a halogen, carbonyl group, —NH₂, —CN, —OH, —C₁-C₆ haloalkyl, —C₁-C₆ alkoxy, —C₁-C₆ haloalkoxy, —C₁-C₆ alkylamino, —C₃-C₆ cycloalkyl or —C₃-C₆ cycloheteroalkyl.

34. The compound or the pharmaceutically acceptable salt of claim 30, wherein the compound is described as Formula II or Formula III: 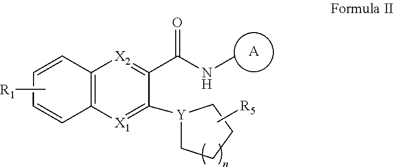
wherein X₁, X₂ are independently selected from C or N, and at least X₁ is N;
R₁ is independently selected from hydrogen, halogen, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃; wherein R₂ and R₃ are independently selected from hydrogen, —NH₂, and —C₁-C₃ alkyl;
Y is selected from CH or N;
R₅ is selected from hydrogen, halogen, —NH₂, —CN, OH, —C₁-C₆ alkyl, —C₁-C₆ haloalkyl, —C₁-C₆ alkoxy, —C₁-C₆ haloalkoxy, —C₁-C₆ alkylamino;
n is an integer ranging from 0 to 6;
ring A is selected from a six-membered aryl or heteroaryl containing 0-3 nitrogen atoms, wherein the aryl or heteroaryl is optionally substituted by hydrogen, halogen, —NH₂, —CN, —OH, —C₁-C₆ alkyl, carbonyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃ or —POR₂R₃; wherein R₂ and R₃ are independently selected from hydrogen, —NH₂, —NHCH₃, and —C₁-C₃ alkyl; 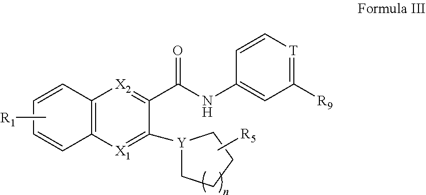
in Formula III, X₁ and X₂ are independently selected from C or N, and at least X₁ is N;
R₁ is independently selected from hydrogen, halogen, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃; wherein R₂ and R₃ are independently selected from hydrogen, —NH₂, and —C₁-C₃ alkyl;
T is CR₆ or N;
R₆ is hydrogen, halogen or —C₁-C₆ alkyl;
R₉ is hydrogen, halogen, —NH₂, —CN, OH, —C₁-C₆ alkyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃, or —POR₂R₃;
R₂ and R₃ are independently selected from hydrogen, —NH₂, —NHCH₃, —C₁-C₃ alkyl, or R₂ and R₃ together with phosphorus form a three- to eight-membered ring;
R₅ is selected from hydrogen, halogen, —NH₂, —CN, —OH, —C₁-C₆ alkyl, —C₁-C₆ haloalkyl, —C₁-C₆ alkoxy, —C₁-C₆ haloalkoxy, —C₁-C₆ alkylamino;
n is an integer ranging from 0 to 6;
preferably, in Formula III, R₁ is independently selected from hydrogen, halogen, —C₁-C₆ alkyl, —C₁-C₆ haloalkyl, —C₁-C₆ alkoxy or —C₁-C₆ haloalkoxy;
preferably, wherein R₁ is independently selected from hydrogen, halogen, —CH₃, —OCH₃;
preferably, wherein R₁ is independently selected from hydrogen or halogen.

35. The compound or the pharmaceutically acceptable salt of claim 34, wherein in Formula II, the ring A is 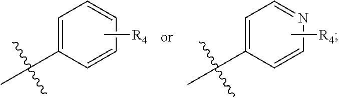
wherein R₄ is hydrogen, halogen, —NH₂, —CN, OH, —C₁-C₆ alkyl, —C₁-C₆ haloalkyl, —C₁-C₆ alkoxy, —C₁-C₆ haloalkoxy, —C₁-C₆ alkylamino, —SO₂R₂, —S(O)(NH)R₂, —COR₂, —CONR₂R₃, or —POR₂R₃;
R₂ and R₃ are independently selected from hydrogen, —NH₂, —NHCH₃, —C₁-C₃ alkyl, or R₂ and R₃ together with phosphorus form a three- to eight-membered ring.

36. The compound or the pharmaceutically acceptable salt of claim 34, wherein in Formula III, Y is a nitrogen and n is an integer from 1 to 4;
preferably, n is 3.

37. The compound or the pharmaceutically acceptable salt of claim 34, wherein the compound of Formula III is described as Formula IV or Formula V: 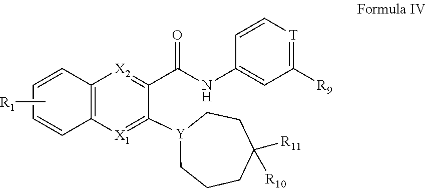
wherein R₁₀ and R₁₁ are independently selected from hydrogen or halogen;
X₁, X₂, T, R₁ and R₉ are defined as described above; 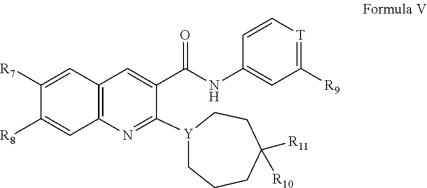
wherein R₇, R₈ are independently selected from hydrogen, halogen, —CH₃, —OCH₃;
R₉ is selected from —SO₂R₂, —S(O)(NH)R₂, —COR₂, —CONR₂R₃;
R₂ and R₃ are independently selected from hydrogen, —NH₂, —NHCH₃, —C₁-C₃ alkyl;
T is CR₆ or N;
R₆ is hydrogen, halogen or —C₁₋₆ alkyl;
R₁₀ and R₁₁ are independently selected from hydrogen or halogen.

38. The compound or the pharmaceutically acceptable salt of claim 37, wherein in Formula IV, R₁ is independently selected from hydrogen, halogen, —C₁-C₆ alkyl, —C₁-C₆ haloalkyl, —C₁-C₆ alkoxy or —C₁-C₆ haloalkoxy;
preferably, wherein R₁ is independently selected from hydrogen, halogen, —CH₃, —OCH₃;
preferably, wherein R₁ is independently selected from hydrogen or halogen.

39. The compound or the pharmaceutically acceptable salt of claim 34, wherein in Formula III, X₁ is N and X₂ is C;
or, wherein X₁ is N and X₂ is N.

40. The compound or the pharmaceutically acceptable salt of claim 34, wherein in Formula III, R₆ is hydrogen or halogen.

41. The compound or the pharmaceutically acceptable salt of claim 34, wherein in Formula III, R₉ is —SO₂R₂, —S(O)(NH)R₂, —COR₂ or —CONR₂R₃, wherein R₂ and R₃ are independently selected from hydrogen, —NH₂, —NHCH₃, and —C₁-C₃ alkyl.

42. The compound or the pharmaceutically acceptable salt of claim 37, wherein in Formula V, R₉ is selected from —SO₂NH₂, —CONH₂;
preferably, wherein R₉ is —SO₂NH₂.

43. The compound or the pharmaceutically acceptable salt of claim 37, wherein in Formula V, R₇ and R₈ are independently selected from hydrogen or halogen.

44. The compound or the pharmaceutically acceptable salt of claim 37, wherein in Formula V, R₆ is H.

45. The compound or the pharmaceutically acceptable salt of claim 37, wherein in Formula V, R₁₀ and R₁₁ are halogen.

46. The compound or the pharmaceutically acceptable salt of claim 30, wherein the compound or the pharmaceutically acceptable salt is selected from: 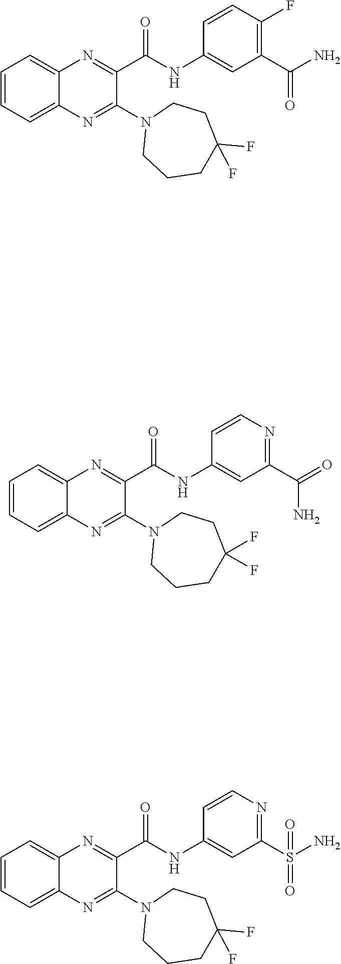 [SMILES: NC(=O)c1cc(NC(=O)c2nc3ccccc3nc2N2CCCC(F)(F)CC2)ccc1F ; NC(=O)c1cc(NC(=O)c2nc3ccccc3nc2N2CCCC(F)(F)CC2)ccn1 ; NS(=O)(=O)c1cc(NC(=O)c2nc3ccccc3nc2N2CCCC(F)(F)CC2)ccn1] 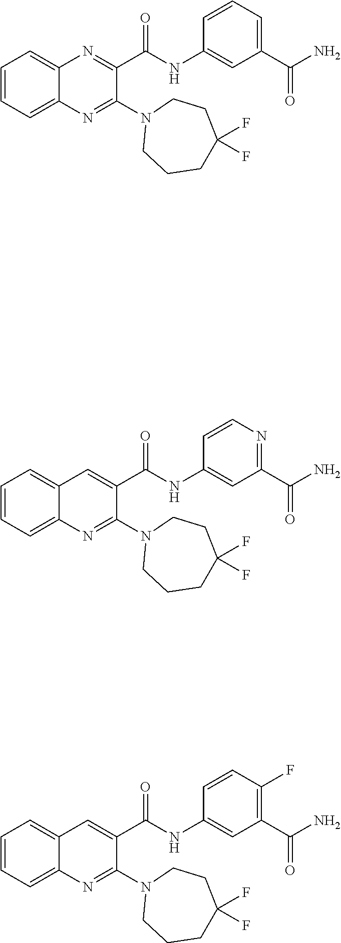 [SMILES: NC(=O)c1cccc(NC(=O)c2nc3ccccc3nc2N2CCCC(F)(F)CC2)c1 ; NC(=O)c1cc(NC(=O)c2cc3ccccc3nc2N2CCCC(F)(F)CC2)ccn1 ; NC(=O)c1cc(NC(=O)c2cc3ccccc3nc2N2CCCC(F)(F)CC2)ccc1F] 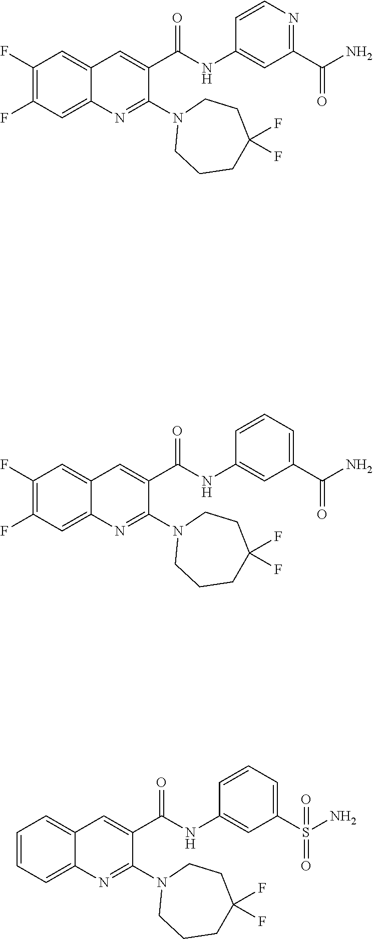 [SMILES: NC(=O)c1cc(NC(=O)c2cc3cc(F)c(F)cc3nc2N2CCCC(F)(F)CC2)ccn1 ; NC(=O)c1cccc(NC(=O)c2cc3cc(F)c(F)cc3nc2N2CCCC(F)(F)CC2)c1 ; NS(=O)(=O)c1cccc(NC(=O)c2cc3ccccc3nc2N2CCCC(F)(F)CC2)c1] 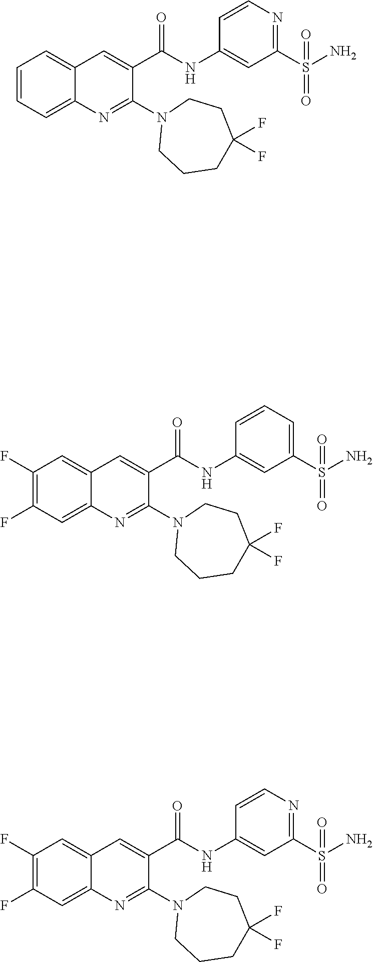 [SMILES: NS(=O)(=O)c1cc(NC(=O)c2cc3ccccc3nc2N2CCCC(F)(F)CC2)ccn1 ; NS(=O)(=O)c1cccc(NC(=O)c2cc3cc(F)c(F)cc3nc2N2CCCC(F)(F)CC2)c1 ; NS(=O)(=O)c1cc(NC(=O)c2cc3cc(F)c(F)cc3nc2N2CCCC(F)(F)CC2)ccn1] 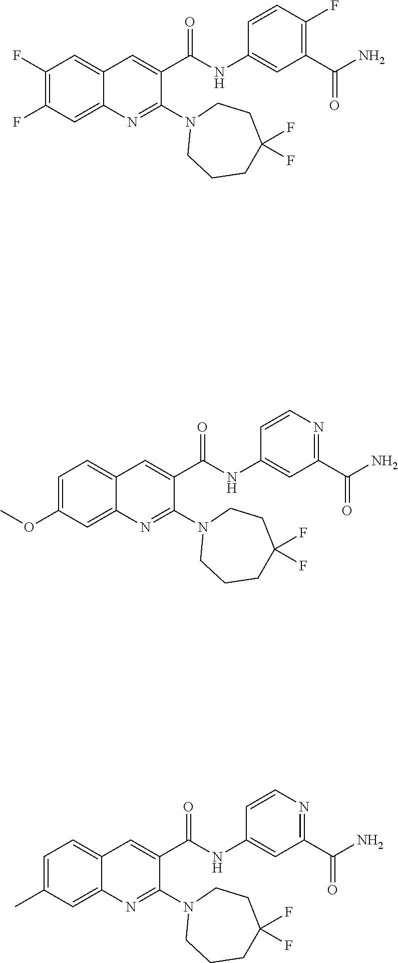 [SMILES: NC(=O)c1cc(NC(=O)c2cc3cc(F)c(F)cc3nc2N2CCCC(F)(F)CC2)ccc1F ; COc1ccc2cc(C(=O)Nc3ccnc(C(N)=O)c3)c(N3CCCC(F)(F)CC3)nc2c1 ; Cc1ccc2cc(C(=O)Nc3ccnc(C(N)=O)c3)c(N3CCCC(F)(F)CC3)nc2c1] 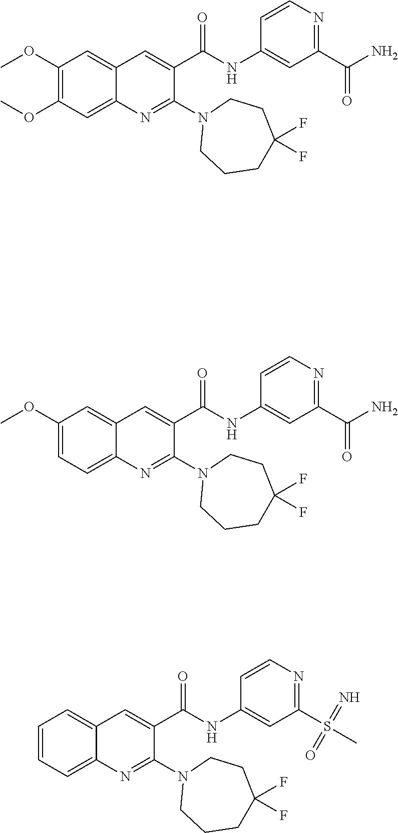 [SMILES: COc1cc2cc(C(=O)Nc3ccnc(C(N)=O)c3)c(N3CCCC(F)(F)CC3)nc2cc1OC ; COc1ccc2nc(N3CCCC(F)(F)CC3)c(C(=O)Nc3ccnc(C(N)=O)c3)cc2c1 ; CS(=N)(=O)c1cc(NC(=O)c2cc3ccccc3nc2N2CCCC(F)(F)CC2)ccn1] 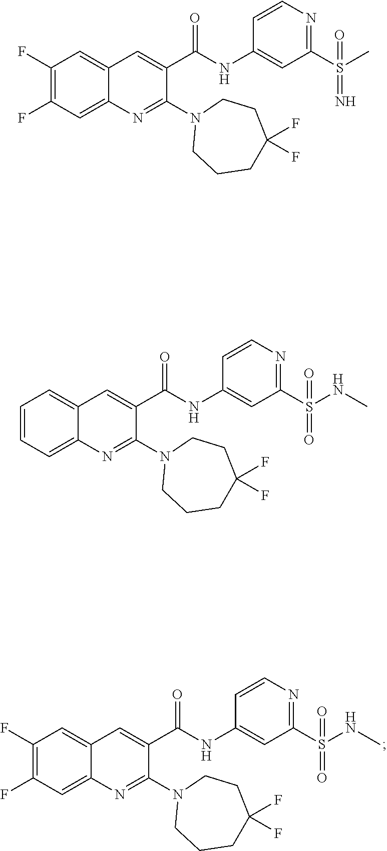 [SMILES: CS(=N)(=O)c1cc(NC(=O)c2cc3cc(F)c(F)cc3nc2N2CCCC(F)(F)CC2)ccn1 ; CNS(=O)(=O)c1cc(NC(=O)c2cc3ccccc3nc2N2CCCC(F)(F)CC2)ccn1 ; CNS(=O)(=O)c1cc(NC(=O)c2cc3cc(F)c(F)cc3nc2N2CCCC(F)(F)CC2)ccn1]
preferably, the compound or the pharmaceutically acceptable salt is selected from:

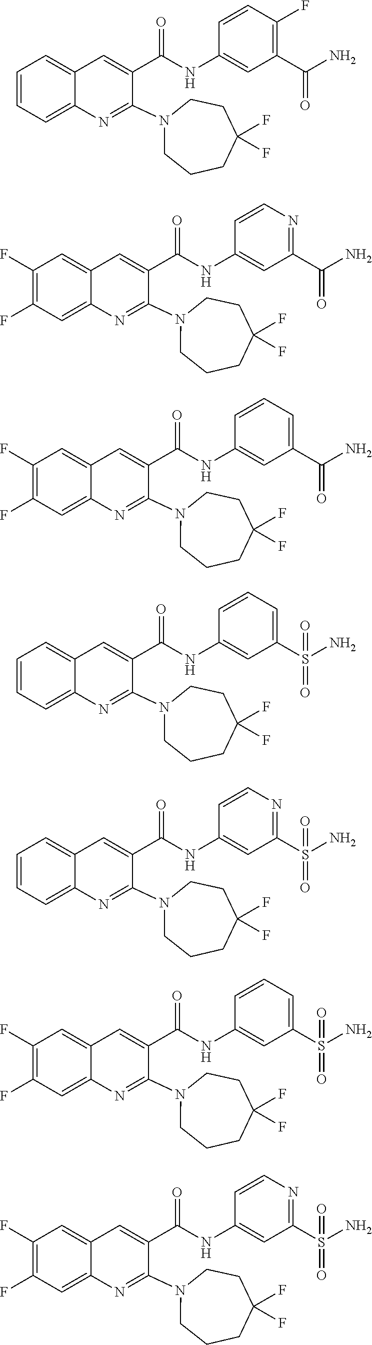

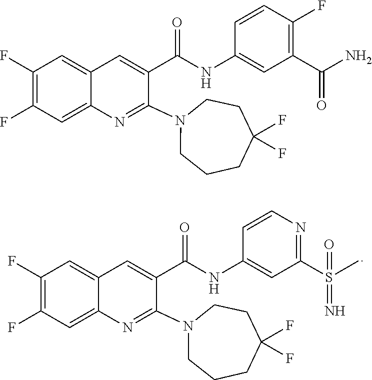 [SMILES: NC(=O)c1cc(NC(=O)c2cc3cc(F)c(F)cc3nc2N2CCCC(F)(F)CC2)ccc1F ; CS(=N)(=O)c1cc(NC(=O)c2cc3cc(F)c(F)cc3nc2N2CCCC(F)(F)CC2)ccn1]

47. A pharmaceutical composition comprising the compound or the pharmaceutically acceptable salt of claim 30, and a pharmaceutically acceptable excipient.

48. A method for preventing or treating pain, comprising administering the compound or the pharmaceutically acceptable salt of claim 30 to a patient.

49. The method of claim 38, wherein the pain is chronic pain, intestinal pain, neuropathic pain, musculoskeletal pain, acute pain, inflammatory pain, cancer pain, primary pain, post-operative pain, visceral pain, multiple sclerosis, Charcot-Marie-Tooth syndrome, incontinence or arrhythmia.
